(19) United States
(12) Patent Application Publication
(10) Pub. No.: US 2023/0077068 A1
(43) Pub. Date: Mar. 9, 2023

(54) MODIFICATION OF EXISTING VALVULAR STRUCTURES FOR PROSTHETIC HEART VALVE IMPLANTATION

(71) Applicant: Edwards Lifesciences Corporation, Irvine, CA (US)

(72) Inventors: Ilan Tamir, Irvine, CA (US); David Maimon, Atlit (IL); Hanoch Cohen-Tzemach, Holon (IL); Adva Alon, Binyamina (IL); Amir Keret, Tirat Hacarmel (IL); Eyal Leiba, N. Misgav (IL); Boaz Manash, Givat Ada (IL); Sean Chow, Irvine, CA (US); Ziv Yohanan, Kfar Hahoresh (IL); Shay Zarneger, Or Akiva (IL)

(21) Appl. No.: 17/932,654

(22) Filed: Sep. 15, 2022

Related U.S. Application Data
(63) Continuation of application No. PCT/US2021/022664, filed on Mar. 17, 2021.
(60) Provisional application No. 63/031,519, filed on May 28, 2020.
(60) Provisional application No. 62/990,734, filed on Mar. 17, 2020.

(51) Int. Cl. A61F 2/24; A61B 17/3205; A61B 18/14; A61B 17/32; A61B 17/34

(52) U.S. Cl. CPC A61F 2/2427; A61B 17/32056; A61F 2/2433; A61B 18/1492; A61B 17/320016; A61B 17/3417; A61B 2017/00867

(57) ABSTRACT

Methods and tools for implanting prosthetic heart valves and modifying leaflets of an existing valvular structure in a subject are disclosed herein. Prior to or during implantation of the prosthetic heart valve within the existing valvular structure, each tool can be provided in the ascending aorta (or equivalent thereof) of a subject and can be used to pierce, lacerate, slice, tear, cut or otherwise modify a leaflet or commissure of the existing valvular structure. The existing valvular structure can be a native aortic valve or other native heart valve, or a previously-implanted prosthetic heart valve. The modification can avoid, or at least reduce the likelihood of, issues that leaflets of the existing valvular structure might otherwise cause once the prosthetic heart valve has been fully installed, for example, obstruction of blood flow to the coronary arteries and/or improper valve mounting due to a non-circular cross-section.

### CROSS REFERENCE TO RELATED APPLICATION

This application is a continuation of PCT application no. PCT/US2021/022664 filed on Mar. 17, 2021, which application claims the benefit of U.S. Provisional Application No. 62/990,734, entitled “Modification Of Existing Valvular Structures For Maintaining Coronary Access After Prosthetic Heart Valve Implantation” filed Mar. 17, 2020, and U.S. Provisional Application No. 63/031,519, entitled “Modification of Existing Valvular Structures for Prosthetic Heart Valve Implantation,” filed May 28, 2020, each of these applications being incorporated by reference herein in its entirety.

### FIELD

The present disclosure relates to prosthetic heart valves, and to methods and devices for modifying existing valvular structures (e.g., leaflets or commissures of a native heart valve or previously-implanted prosthetic valve) prior to or during implantation of a prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

As surgical approaches for valve replacement become available for younger patients, patient lifetime may exceed the corresponding lifetime of the implanted prosthetic valve. Valve-in-valve (ViV) procedures have been developed to mount a new prosthetic valve within the previously-implanted prosthetic valve. However, such procedures may pose a risk of coronary artery obstruction. In particular, the leaflets of the previously-implanted prosthetic valve may block the coronary ostia or otherwise inhibit blood flow through the frame of the new prosthetic valve to the coronary ostia. A similar problem may occur when a prosthetic valve is percutaneously expanded within a native aortic valve. In some instances, the native aortic valve may be abnormal, for example, a bicuspid aortic valve (BAV) having two leaflets. The BAV leaflets may be stiffer than normal and/or define a non-circular geometry, which may compromise the ability for a prosthetic valve having a cylindrical geometry to be successfully implanted therein. For example, a patient having a prosthetic heart valve implanted within the non-circular geometry of an unmodified BAV may be at an increased risk for annular rupture and/or reduced hemodynamic performance.

Existing methods, which rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

### SUMMARY

Described herein are embodiments of tools and methods for implanting prosthetic heart valves and modifying leaflets of an existing valvular structure in a subject's heart. The tools can be provided in the ascending aorta of a subject (or an equivalent thereof) and can be used to pierce, lacerate, slice, cut or otherwise modify a leaflet or commissure of the existing valvular structure. In some embodiments, the existing valvular structure can be a native aortic valve (e.g., normal or abnormal, such as bicuspid aortic valve (BAV)) or a previously implanted prosthetic heart valve. In some embodiments, the modification of the leaflets can occur prior to installation of a new prosthetic heart valve within the existing valvular structure. In other embodiments, the modification of the leaflets can occur during installation of the new prosthetic heart valve, for example, with the prosthetic heart valve partially expanded within the existing valvular structure. Optionally, each tool can have one or more filters that trap particulate or other debris that released from the existing valvular structure during the modification. The modification can avoid, or at least reduce the likelihood of, issues that leaflets of the existing valvular structure might otherwise cause once the prosthetic heart valve is fully installed, for example, obstruction of blood flow to the coronary arteries and/or improper valve mounting due to a non-circular cross-section. In other embodiments, the existing valvular structure can be one of the other native heart valves, e.g., pulmonary, tricuspid, or mitral valve, or a prosthetic heart valve previously implanted therein.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

FIG. 1A shows a cross-sectional view of a prosthetic heart valve implanted in the native aortic valve annulus.

FIG. 1B shows the implanted prosthetic heart valve of FIG. 1A as viewed from the ascending aorta.

FIG. 2A shows a first exemplary tool for cutting leaflets, according to one or more embodiments of the disclosed subject matter.

FIGS. 2B-2D are simplified side views of a positioning stage, piercing stage, and cutting stage, respectively, in cutting of a leaflet of an existing valvular structure using the first tool of FIG. 2A.

FIG. 2E shows the existing valvular structure after cutting by the first tool of FIG. 2A, as viewed from the ascending aorta.

FIGS. 3A-3B show cross-sectional and side views, respectively, of a second exemplary tool for cutting leaflets, according to one or more embodiments of the disclosed subject matter.

FIG. 4A is a simplified perspective view of a positioning stage in cutting of a commissure of an existing valvular structure using a third exemplary tool for cutting commissures, according to one or more embodiments of the disclosed subject matter.

FIGS. 4B-4D show first side, second side, and bottom views, respectively, of a head portion of the third tool for cutting commissures.

FIG. 4E is a close-up perspective view of the head portion of FIGS. 4A-4D during a cutting stage in cutting of the leaflet of the existing valvular structure.

FIG. 4F shows a first side view of an alternative configuration for the head portion of the third exemplary tool for cutting commissures.

FIG. 5A is a simplified perspective view of a positioning stage in cutting of a commissure of an existing valvular structure using a fourth exemplary tool for cutting commissures, according to one or more embodiments of the disclosed subject matter.

FIG. 5B is a close-up perspective view of a head portion of the fourth tool for cutting commissures.

FIGS. 5C-5D are side views of a gripping mechanism and a cutting mechanism, respectively, of the head portion of the fourth tool for cutting commissures.

FIG. 6A shows a fifth exemplary tool for cutting multiple commissures simultaneously, according to one or more embodiments of the disclosed subject matter.

FIGS. 6B-6C are simplified perspective views of a positioning stage and cutting stage, respectively, in the simultaneous cutting of commissures of an existing valvular structure using the fifth tool of FIG. 6A.

FIGS. 6D-6E show first and second stages, respectively, in retraction of the fifth tool of FIG. 6A into a catheter for removal from a subject.

FIGS. 7A-7B shows a sixth exemplary tool for cutting leaflets, in a first contracted state and in a second expanded state, respectively, according to one or more embodiments of the disclosed subject matter.

FIGS. 7C-7D are simplified close-up side views of a positioning stage and a cutting stage, respectively, in cutting of a leaflet of an existing valvular structure using the sixth tool of FIGS. 7A-7B.

FIG. 7E is a close-up side view showing an exemplary interaction between the cutting and positioning elements of the sixth tool of FIGS. 7A-7B during the cutting.

FIGS. 7F-7G shows exemplary cross-sectional geometries for the cutting element of the sixth tool of FIGS. 7A-7B.

FIGS. 7H-7I are simplified views from the ascending aorta of positioning and cutting stages, respectively, in simultaneous cutting of multiple leaflets of an existing valvular structure using a variation of the sixth exemplary tool for cutting leaflets, according to one or more embodiments of the disclosed subject matter.

FIGS. 7J, 7K, and 7M are close-up side views showing alternative configurations for the cutting and positioning elements of the sixth exemplary tools of FIGS. 7A-7I.

FIG. 7L is a close-up cross-sectional view showing exemplary snap fit features for the cutting and positioning elements of the sixth exemplary tools of FIGS. 7A-7I.

FIG. 8A is a simplified close-up side view of a positioning stage in cutting a leaflet of an existing valvular structure using a seventh exemplary tool with tubular member, according to one or more embodiments of the disclosed subject matter.

FIG. 8B is a simplified side view of a variation of the seventh exemplary tool of FIG. 8A having multiple cutting elements for cutting multiple leaflets.

FIGS. 9A-9B are side and perspective views, respectively, of an eighth exemplary tool for cutting leaflets, according to one or more embodiments of the disclosed subject matter.

FIGS. 9C and 9E are simplified side views of a positioning stage and cutting stage, respectively, in cutting of a leaflet of an existing valvular structure using the eighth tool of FIGS. 9A-9B.

FIGS. 9D and 9F are views from the ascending aorta of the positioning and cutting stages of FIGS. 9C and 9E, respectively.

FIG. 10A is a side view of a ninth exemplary tool for cutting leaflets, according to one or more embodiments of the disclosed subject matter.

FIGS. 10B-10C are cross-sectional views of the ninth tool of FIG. 10A in a stowed configuration and deployed configuration, respectively.

FIGS. 10D-10F are perspective cut-away views of the ninth tool of FIG. 10A during a positioning stage in cutting of a leaflet of an existing valvular structure.

FIG. 10G is a perspective cut-away view of the ninth tool of FIG. 10A during a piercing stage in cutting of the leaflet of the existing valvular structure.

FIG. 10H is a perspective cut-away view of the ninth tool of FIG. 10A during a cutting stage in cutting of the leaflet of the existing valvular structure.

FIG. 10I shows the existing valvular structure after cutting by the ninth tool of FIG. 10A, as viewed from the ascending aorta.

FIG. 10J is a perspective cut-away of the ninth tool of FIG. 10A during a retracting stage after cutting of the leaflet of the existing valvular structure.

FIGS. 11A-11B are simplified cross-sectional views of a positioning stage and piercing stage, respectively, in cutting of a leaflet of an existing valvular structure using a tenth exemplary tool, according to one or more embodiments of the disclosed subject matter.

FIG. 11D is a close-up cross-sectional view of an alternative configuration of a piercing member for the tenth exemplary tool for cutting of the leaflet of the existing valvular structure.

FIGS. 11C and 11E illustrate close-up cross-sectional views of alternative configurations of an end of a delivery shaft for the tenth exemplary tool for cutting of the leaflet of the existing valvular structure.

FIGS. 12A-12C are simplified cross-sectional views of a positioning stage, clamping stage, and cutting stage, respectively, in cutting of a leaflet of an existing valvular structure using an eleventh exemplary tool, according to one or more embodiments of the disclosed subject matter.

FIG. 12D is a side view of an outer shaft of an exemplary construction of the eleventh tool.

FIGS. 12E-12F are first and second side views of an inner shaft of an exemplary construction of the eleventh tool.

FIG. 12G is a side view of the assembled inner and outer shafts of the exemplary construction of FIGS. 12D-12F positioned for the clamping stage.

FIG. 12H is a side view of the assembled inner and outer shafts of the exemplary construction of FIGS. 12D-12F positioned for the cutting stage.

FIG. 12I is a side view of a cutting shaft of another exemplary construction of an eleventh tool.

FIGS. 12J-12M are side views of the assembled inner, outer, and cutting shafts of the eleventh tool construction of FIG. 12I, positioned for a positioning stage, a clamping stage, a cutting shaft actuation stage, and a cutting stage, respectively.

FIG. 13A is a simplified cross-sectional view of a twelfth exemplary tool for cutting a leaflet of an existing valvular structure, according to one or more embodiments of the disclosed subject matter.

FIG. 13B is a simplified cross-sectional view of a variation of the twelfth exemplary tool for cutting a leaflet of an existing valvular structure.

FIGS. 13C and 13D-13E are simplified side and top-down cross-sectional views of another variation of the twelfth exemplary tool for cutting a leaflet of an existing valvular structure.

FIG. 14A is a perspective view of a thirteenth exemplary tool for cutting a leaflet of an existing valvular structure, according to one or more embodiments of the disclosed subject matter.

FIGS. 14B-14C illustrate simplified cross-sections at different positions along the thirteenth tool of FIG. 14A.

FIG. 14D is a perspective view of the thirteenth tool of FIG. 14A positioned for a leaflet positioning stage.

FIGS. 14E-14F are perspective view and close-up perspective view, respectively, of the thirteenth tool of FIG. 14A positioned for a first cutting stage.

FIG. 14G is a perspective view of the thirteenth tool of FIG. 14A positioned for pulling the leaflet for further cutting.

FIG. 15A is a perspective view of a delivery assembly for a mechanically-expandable prosthetic heart valve, according to one or more embodiments of the disclosed subject matter.

FIG. 15B is a perspective view of the prosthetic heart valve of FIG. 15A.

FIG. 15C is a perspective view of the prosthetic heart valve of FIG. 15A, without the valvular structure and with the frame of the valve in a radially expanded configuration.

FIG. 15D is a side view of the prosthetic heart valve of FIG. 15A in a radially compressed configuration.

FIGS. 15E-15F are detail and cross-sectional views, respectively, of an actuation mechanism of the prosthetic heart valve of FIG. 15A.

FIGS. 16A-16C depict stages of an exemplary implantation procedure where the prosthetic valve is partially expanded within the native aortic valve of a subject.

FIG. 16D is a detailed view of an exemplary leaflet laceration stage during partial valve expansion.

FIGS. 16E-16F depict further stages of the exemplary implantation procedure after leaflet laceration.

FIGS. 17A-17C are simplified side views of a positioning, advancing, and cutting stages, respectively, in cutting of leaflets of an existing valvular structure using a fourteenth exemplary tool, according to one or more embodiments of the disclosed subject matter.

FIGS. 18A-18B are simplified views from the ascending aorta of a native normal aortic valve before and after laceration, respectively.

FIGS. 19A-19B are simplified views from the ascending aorta of a native bicuspid aortic valve before and after laceration, respectively.

FIG. 20A is a simplified cross-sectional view of a fifteenth exemplary tool for cutting a leaflet of an existing valvular structure, according to one or more embodiments of the disclosed subject matter.

FIGS. 20B-20E are simplified side views of positioning, advancing, cutting, and withdrawal stages, respectively, in cutting of a leaflet of a native valvular structure using the fifteenth tool, according to one or more embodiments of the disclosed subject matter.

FIGS. 21A-21B are simplified side views of cutting and withdrawal stages, respectively, in cutting of a leaflet of a previously implanted prosthetic heart valve using the fifteenth tool, according to one or more embodiments of the disclosed subject matter.

FIGS. 22A-22B are exploded and assembled views, respectively, of a sixteenth exemplary tool for cutting a leaflet of an existing valvular structure, according to one or more embodiments of the disclosed subject matter.

FIGS. 23A-23B, 23D, and 23F are side views of a positioning, engagement, cutting, and withdrawal stages, respectively, in cutting of a leaflet of an existing valvular structure using the sixteenth tool, according to one or more embodiments of the disclosed subject matter.

FIGS. 23C and 23E are magnified cross-sectional views of the engagement of FIG. 23B and the cutting of FIG. 23D, respectively.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present, or problems be solved. The technologies from any example can be combined with the technologies described in any one or more of the other examples.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery system or apparatus that is outside the subject, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed embodiments are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and “or.”

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

### Examples of the Disclosed Technology

Described herein are tools and methods for implanting prosthetic heart valves and modifying leaflets of an existing valvular structure in a subject. Prior to or during implantation of the prosthetic heart valve within the existing valvular structure, each tool can be provided in the ascending aorta (or an equivalent thereof) of a subject and can be used to pierce, lacerate, slice, tear, cut or otherwise modify a leaflet or commissure of the existing valvular structure. In some embodiments, the existing valvular structure can be a native aortic valve (e.g., normal or abnormal, such as bicuspid aortic valve (BAV)) or a prosthetic heart valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries and/or improper mounting due to a non-circular valve cross-section. In other embodiments, the existing valvular structure can be one of the other native heart valves (e.g., pulmonary, tricuspid, or mitral valves) or a prosthetic heart valve implanted therein.

In various embodiments described herein, tools and methods may be deployed or performed within a subject. Subjects include (but are not limited to) medical patients, veterinary patients, animal models, cadavers, and simulators of the cardiac and vasculature system (e.g., anthropomorphic phantoms and explant tissue). Accordingly, various embodiments are directed to methods for medical procedures, practice of medical procedures, and/or training of medical procedures. Simulators may include a whole or partial vasculature system, a whole or partial heart, and/or whole or partial components of the vasculature system (e.g., who or partial ascending aorta). A native tissue and/or component refers to the original tissue and/or component of the patient, animal model, cadaver, or simulator.

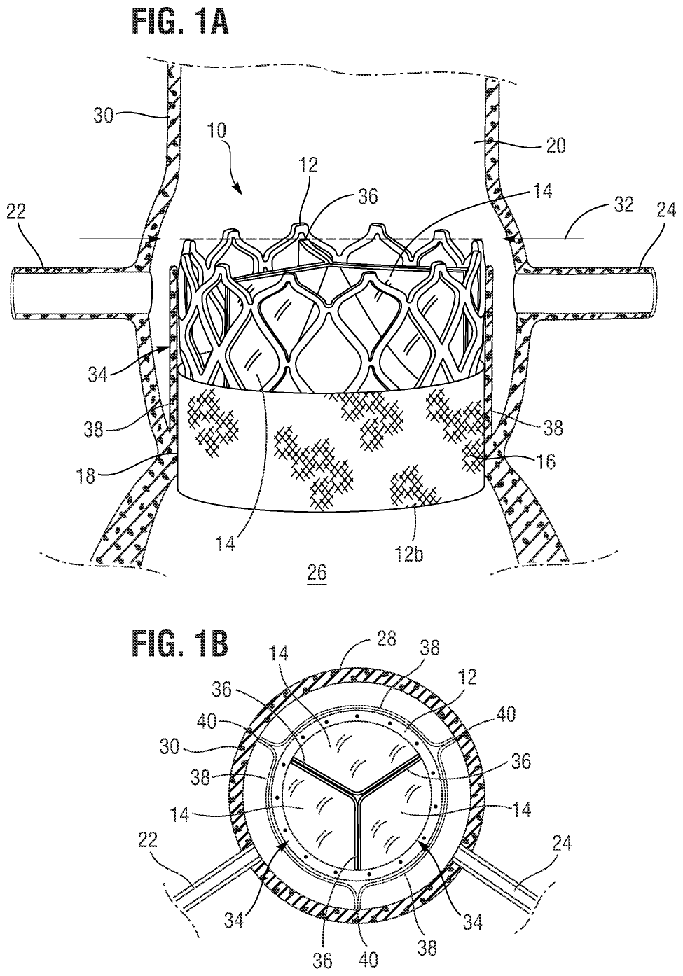

FIGS. 1A-1B shows an exemplary prosthetic heart valve 10, according to one or more embodiments of the disclosed subject matter. The prosthetic heart valve 10 can be radially compressible/expandable between a compressed configuration for delivery into a subject and an expanded configuration for mounting (e.g., as shown in FIG. 1A). In particular embodiments, the prosthetic heart valve 10 can be implanted within the native aortic annulus 18.

The prosthetic heart valve 10 can include an annular stent or frame 12. The frame 12, or components thereof (e.g., struts and/or fasteners), can be made of any of various suitable plastically-expandable materials (e.g., stainless steel, etc.) or self-expanding materials (e.g., nickel titanium alloy (NiTi), such as nitinol), as known in the art. Suitable plastically-expandable materials that can be used to form the frame 12 include, without limitation, stainless steel, biocompatible, high-strength alloys (e.g., a cobalt-chromium or a nickel-cobalt-chromium alloys), polymers, or combinations thereof. In particular embodiments, frame 12 is made of a nickel-cobalt-chromium-molybdenum alloy, such as MP35N® alloy (SPS Technologies, Jenkintown, Pa.), which is equivalent to UNS R30035 alloy (covered by ASTM F562-02). MP35N® alloy/UNS R30035 alloy comprises 35% nickel, 35% cobalt, 20% chromium, and 10% molybdenum, by weight.

When constructed of a plastically-expandable material, the frame 12 (and thus the prosthetic valve 10) can be crimped to a radially collapsed configuration on a delivery catheter and then expanded inside a subject by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

In some embodiments, struts of the frame 12 are pivotable or bendable relative to each other to permit radial expansion and contraction of the frame 12. For example, the frame 12 can be formed (e.g., via laser cutting, electroforming or physical vapor deposition) from a single piece of material (e.g., a metal tube). In other embodiments, the frame 12 can be constructed by forming individual components (e.g., the struts and fasteners of the frame) and then mechanically assembling and connecting the individual components together. Further details regarding the construction of the frame 12 and the prosthetic heart valve 10 are described in U.S. Pat. Nos. 9,393,110, 10,603,165, and 10,806,573, U.S. Patent Application Publication Nos. 2018/0344456, 2019/0365530, 2020/0188099, and 2020/0390547, and International Publication Nos. WO/2020/081893 and WO/2021/003167, all of which are incorporated herein by reference.

The frame 12 can have a first axial end and a second axial end. In the depicted embodiment, the first axial end (e.g., facing the ascending aorta 20 near sinotubular junction level 32) can be an outflow end, and the second axial end (e.g., facing the left ventricle 26 near aortic root 18) can be an inflow end. In some embodiments, the outflow end can be coupled to a delivery apparatus for delivering the prosthetic valve to the implantation site. Alternatively, the prosthetic valve 10 can be radially crimped on an inflatable balloon of a delivery apparatus for delivery to the implantation site. Implanting the prosthetic heart valve 10 within the native aortic valve can be via a transfemoral, retrograde delivery approach. Thus, in the delivery configuration of the prosthetic heart valve, the outflow end is the proximal-most end of the prosthetic valve. In other embodiments, the inflow end can be the proximal-most end of the prosthetic heart valve in the delivery configuration, depending on the particular native valve being replaced and the delivery technique that is used (e.g., trans-septal, transapical, etc.). In some cases, the inflow end can be coupled to the delivery apparatus in the delivery configuration.

The prosthetic valve 10 also includes a valvular structure configured for allowing blood flow through the frame 12 in one direction. The valvular structure can be configured to regulate the flow of blood through the prosthetic heart valve 10 from the inflow end to the outflow end. The valvular structure can include, for example, a leaflet assembly formed by one or more leaflets 14 (three leaflets illustrated in FIGS. 1A-1B) made of a flexible material. Adjacent leaflets 14 can be arranged together to form commissures 36 that are coupled (directly or indirectly) to respective portions of the frame 12, thereby securing at least a portion of the leaflet assembly to the frame 12. The leaflets 14 can be made from in whole or part, biological material, bio-compatible synthetic materials, or other such materials. Suitable biological material can include, for example, bovine pericardium (or pericardium from other sources). Further details regarding transcatheter prosthetic heart valves, including the manner in which the valvular structure can be coupled to the frame 12 of the prosthetic heart valve 10, can be found, for example, in U.S. Pat. Nos. 6,730,118, 7,393,360, 7,510,575, 7,993,394, 8,652,202, and 9,393,110, and U.S. Patent Application Publication Nos. 2018/0325665 and 2019/0365530, all of which are incorporated herein by reference in their entireties.

The prosthetic heart valve 10 can also include one or more skirts or sealing members. For example, the prosthetic heart valve 10 can include an inner skirt mounted on the inner surface (not shown in FIGS. 1A-1B) of the frame 12 and/or an outer skirt 16 mounted on the outer surface of the frame 12. The inner skirt can be a circumferential inner skirt that spans an entire circumference of the inner surface of the frame 12. The inner skirt can function as a sealing member to prevent or decrease perivalvular leakage (e.g., when the valve is placed at the implantation site) and as an attachment surface to anchor a portion of the leaflets 14 to the frame 12. The outer skirt 16 can function as a sealing member by sealing against the tissue of the native valve annulus 18 and helping to reduce paravalvular leakage past the prosthetic valve 10. The inner and outer skirts can be formed from any of various suitable biocompatible materials, including any of various synthetic materials (e.g., polyethylene terephthalate (PET)) or natural tissue (e.g., pericardial tissue). The inner and outer skirts can be mounted to the frame using sutures, an adhesive, welding, and/or other means for attaching the skirts to the frame. Further details regarding the inner and outer skirts and techniques for assembling the leaflets to the inner skirt and assembling the skirts on the frame are disclosed in U.S. Pat. No. 9,393,110, U.S. Patent Application Publication Nos. 2019/0192296 and 2019/0365530, and International Publication Nos. WO/2020/198273 and WO/2020/159783, all of which are incorporated herein by reference.

In the description of the disclosed embodiments, the methods and devices are described in the context of using a retrograde delivery approach to the native aortic valve. As such, the term “proximal end” of the prosthetic valve (or other device) or a component thereof is used to refer to its outflow end and the term “distal end” of the prosthetic valve (or other device) or a component thereof is used to refer to its inflow end. However, it should be noted if delivered in the opposite direction to the aortic valve (e.g., transapically) the outflow end of the prosthetic valve would be the distal end and the inflow end of the prosthetic valve would be the proximal end during delivery. Thus, in the present application, once a prosthetic valve is implanted at the aortic position, the term “proximal end” is intended to mean “outflow end” and the term “distal end” is intended to mean “inflow end”. Similarly, the terms “distal side” and “distal” as used herein to describe components or parts of the anatomy are intended to mean “upstream side” and “upstream” while the terms “proximal side” and “proximal” as used herein to describe components or parts of the anatomy are intended to mean “downstream side” and “downstream”. Further, any of the methods and devices described herein can be applied to any of the native valves of the heart (the aortic, mitral, tricuspid, and pulmonary valves) or a prosthetic valve previously implanted within any of the native valves of the heart using any known techniques, which can involve approaching a native valve in a retrograde or antegrade direction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. However, ViV procedures may pose an increased risk of obstruction of the coronary arteries 22, 24. In particular, the mounting of the new prosthetic heart valve within the valvular structure of the existing prosthetic heart valve can displace the leaflets of the existing heart valve outwards, thereby obstructing the ostia of the coronary arteries 22, 24. Moreover, since the leaflets of the existing heart valve are disposed outside the frame of the new prosthetic valve, they may cover external surfaces of the frame, thereby creating a substantially impermeable tubular structure that occludes openings 34 in frame 12. In some subject anatomies (e.g., when the outflow of the valve 10 is at the sinotubular junction (STJ) level 32 and the diameter of the valve 10 is similar to the STJ diameter such that the frame 12 touches or is very close to the aortic wall 30 at the STJ level 32), the leaflets of the existing valve structure may compromise the ability for future access into the coronary arteries 22, 24 or perfusion through the valve frame 12 to the coronary arteries 22, 24 during the diastole phase of the cardiac cycle. Similar problems may occur in some subject anatomies when a prosthetic heart valve 10 is percutaneously expanded within a native valve, displacing the native leaflets 38 outward toward the coronary ostia.

To avoid obstruction of blood flow to the coronary arteries 22, 24, the valvular structure of the existing heart valve (whether a native aortic valve or a previously implanted prosthetic valve) can be modified by a tool prior to or during implantation of a new prosthetic heart valve within the existing valvular structure. In embodiments, the valvular structure is modified by piercing, lacerating, tearing, slicing, and/or cutting one or more leaflets 14 (e.g., a free end of the leaflet 14 or a commissure 36 of adjacent leaflets 14) using the tool. The modification thus disrupts the impermeable tubular structure that would otherwise be formed by the existing leaflets 14, thereby allowing blood to flow to the coronary arteries 22, 24. Alternatively or additionally, when the existing valvular structure is a BAV, the modification can allow for improved mounting of the cylindrical prosthetic valve, which would otherwise be compromised by the non-circular geometry of the native BAV.

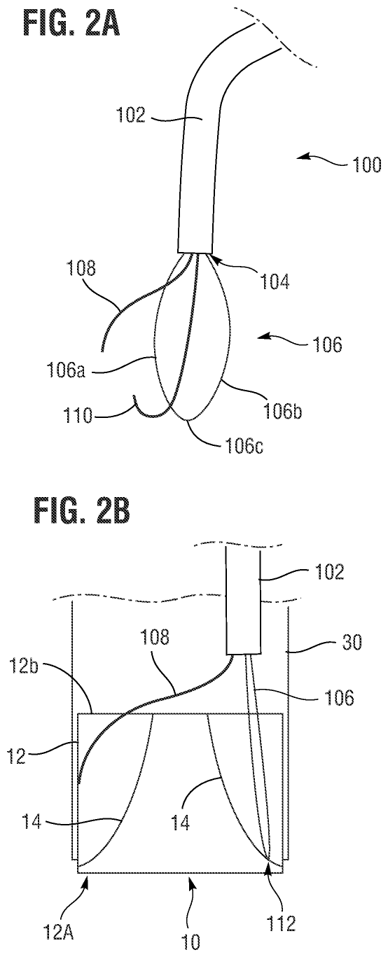

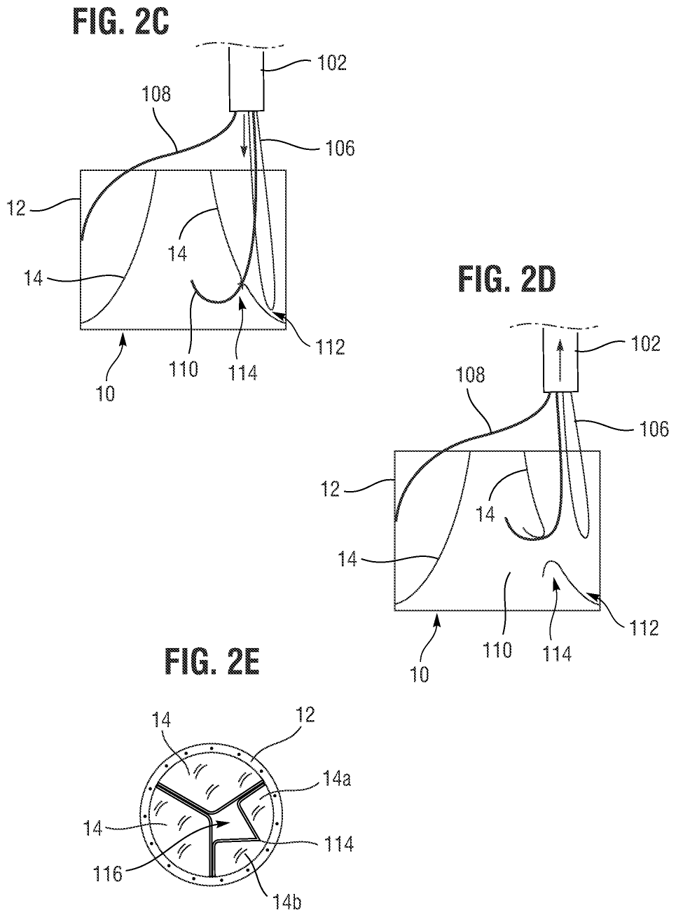

For example, FIG. 2A illustrates a first exemplary tool 100 that can be used to cut a leaflet 14 of an existing valvular structure. The tool 100 comprises a catheter 102 with a distal end 104 configured to be disposed within an ascending aorta of a subject. The catheter 102 contains therein at least three members—a first spacing member 106, a second spacing member 108, and a lacerating member 110. Each of the members 106-110 may be independently maneuvered within catheter 102 and extending from catheter 102, for example, via operation of a handle (not shown) at the proximal region of the catheter 102 by an operator. The catheter 102 in the illustrated embodiment is in the form of a shaft, although in other embodiments described below, the catheter can include multiple shafts, which can be co-axially disposed relative to each other.

Each of the members 106-110 can be extended from the end 104 of the catheter 102 to interact with the existing valvular structure. For example, as shown in FIGS. 2B-2D, the first positioning member 106 can be configured for insertion into a pocket 112 between a leaflet 14 of the existing valvular structure and the frame 12 (or the aortic wall 30 when dealing with the native valve), in order to provide a sufficient spacing for subsequent positioning of the lacerating member 110. The second spacing member 108 can be configured to push against an opposite side of the frame 12 (or the aortic wall 30 when dealing with the native valve) so as to enhance circumferential positioning and alignment of the first positioning member 106 and the lacerating member 110 during cutting of leaflet 14.

Each of the members 106-110 can be a pre-shaped wire, for example, a wire formed of a shape memory alloy such as Nitinol. Thus, as the members 106-110 are advanced out of the end 104 of the catheter 102, end portions thereof can adopt their pre-determined shape. For example, the first positioning member 106 can have a contoured end having a circular shape, oval shape (e.g., spoon-shaped), elliptical shape, or any other shape, so as to increase a region over which the first positioning member 106 contacts a downstream side of the leaflet 14. In some embodiments, the first positioning member 106 can be formed from a plurality of wires, for example, as a pair of wires 106a, 106b that are bent laterally to converge to contact or attach to each other at distal tip 106c. The second spacing member 108 can be bent or curved along its length so as to push on a structure (e.g., valve frame or aortic wall) opposite the first positioning member 106. The lacerating member 110 can be bent at its end portion, for example, to form a hook shape.

FIGS. 2B-2D show various stages for using the first exemplary tool 100 to modify a leaflet 14 of the existing valvular structure to avoid coronary artery obstruction by subsequent implantation of a prosthetic heart valve. Catheter shaft 102 can be advanced to the existing valvular structure from the ascending aorta. The first positioning member 106, second positioning member 108, and lacerating member 110 may be retained within the catheter 102 prior to reaching valve 10. Once the catheter 102 reaches the valve 10 (e.g., the distal end 104 is positioned near valve 10), the first positioning member 106 can be advanced out of the catheter 102 into the pocket 112 formed between the leaflet 14 and the frame 12 of the existing valve 10. For example, the first positioning member 106 can be advanced up to (e.g., into contact with) where the leaflet 14 attaches to the distal end 12a of the frame 12, for example along a scalloped line, as shown in FIG. 2B.

Subsequent to or simultaneous with advancement of the first positioning member 106 out of the catheter 102, the second positioning member 108 can be advanced out of the catheter 102. As noted above, the second positioning member 108 can be bent so at deflect radially outward away from the first positioning member 106 once it leaves the catheter 102, thereby pressing against a side of frame 12 that is opposite the first positioning member 106, as shown in FIG. 2A. The second positioning member 108 thus acts to align the position of the first positioning member 106 and to retain member 106 in position during subsequent lacerating procedures.

With the first 106 and second 108 positioning members in place within valve 10, the lacerating member 110 can then be advanced out of the catheter 102 toward the space formed between a free end of the leaflet 14 and the frame 12 of the valve 10. As noted above, the lacerating member 110 can be bent at its end portion so as to adopt a J-shape or hook-shape once it extends from end 104 of the catheter 102. The first positioning member 106 thus enlarges the gap between the proximal end (e.g., free end) of the leaflet 14 and the frame 12 in order to facilitate positioning of the bent end of the lacerating member 110 on the downstream side of the leaflet 14.

The end portion of the lacerating member 110 can be relatively stiff (e.g., stiffer than other portions of the lacerating member 110 and/or the other members 106-108) and/or have a sharp tip, such that the lacerating member 110 pierces the leaflet 14 at 114, as shown in FIG. 2C. Alternatively or additionally, the lacerating member 110 is configured to cut or lacerate the tissue of leaflet 14 by application of electrical energy (e.g., radiofrequency (RF) energy)) at the end portion of the lacerating member 110. For example, electrical energy can be applied to a distal or mid-portion of the lacerating member 110 that contacts the leaflet 14 such that the lacerating member 110 penetrates the leaflet 14 at 114, as shown in FIG. 2C. The lacerating member 110 can then be moved proximally away from the valve 10 as shown in FIG. 2D, for example, while optionally applying electrical energy thereto, in order to form a gash 116 that splays leaflet 14 into separate portions 14a-14b, as shown in FIG. 2E. For example, the proximal motion of the lacerating member 110 can be by retracting the lacerating member 110 into the catheter 102 while holding the catheter 102 and/or members 106-108 at static positions, retracting all members 106-110 into the catheter 102 while holding the catheter 102 at a static position, retracting the catheter 102 in a proximal direction while maintaining positions of members 106-110 with respect to distal end 104, or any other combination of motions of members 106-110 and catheter 102.

If additional cutting of one or more leaflets 14 of the existing valvular structure is desired, for example, to provide an opening for both coronary arteries 22, 24, then the catheter 102 can be repositioned (e.g., rotated) with respect to the next leaflet after partial or full retraction of members 106-110 into the catheter 102. The cutting of the next leaflet 14 can then be performed in a similar manner as described above for FIGS. 2B-2E. When no further cutting is desired, the members 106-110 can be fully retracted and the catheter 102 retrieved from the subject. As part of the ViV procedure, the new prosthetic heart valve in a crimped state can subsequently be advanced to the existing valvular structure with cut leaflets 14. The new valve is disposed within the valvular structure and expanded, such that the leaflets 14 are disposed on an external surface of the new valve frame. However, the one or more gashes 116 formed in the leaflets 14 allows blood to flow from the outflow end of the new prosthetic valve to the coronary arteries 22, 24 once the ViV procedure is completed. Alternatively or additionally, when the existing valvular structure is a BAV, the one or more gashes formed in the leaflets can allow for improved mounting of the cylindrical prosthetic valve, which would otherwise be compromised by the non-circular geometry of the native BAV.

In some instances, multiple leaflets 14 of the existing valvular structure (e.g., two of the three leaflets of the aortic valve) may require modification to allow for adequate blood flow to reach both coronary arteries after implantation of the new prosthetic valve. Since members 106-110 of the first tool 100 are maneuverable independent of each other, the subsequent modification of another leaflet may require independent retrieving and re-positioning of each member 106-110, which may be tedious and/or time-consuming. In some embodiments, a positioning member can be used to simultaneously reposition the members 106-110 to modify the next leaflet, for example, by simple rotation of the members 106-110 relative to the existing valvular structure while maintaining the relative positions between the members 106-110.

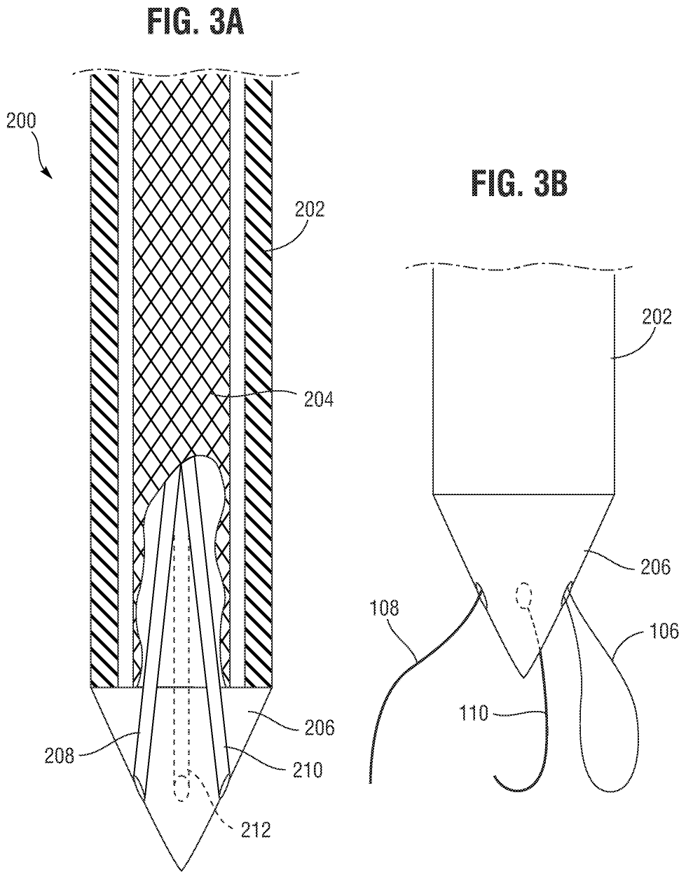

For example, FIGS. 3A-3B illustrates a second exemplary tool 200 that can be used to sequentially cut multiple leaflets 14 of an existing valvular structure. The second tool 200 can have a first positioning member 106, a second spacing member 108, and a lacerating member 110 and can operate in a similar manner as the first tool 100, as described above with respect to FIGS. 2A-2E. However, the second tool 200 can also have a multi-channel positioning member 206 disposed at the distal end of catheter 202. The multi-channel positioning member 206 can have channels 210, 208, and 212 through which the first positioning member 106, second spacing member 108, and lacerating member 110 respectively extend. The positioning member 206 can be rotatable about an axis of the catheter 202 such that the respective outlets of channels 208-212 are reoriented with respect to another leaflet of the existing valvular structure for cutting. The positioning member 206 can have a shape that facilitates insertion into and movement through a blood vessel of the subject. For example, the positioning member 206 can have a conical or tapered shape (e.g., nose-cone shaped) that narrows from its proximal to its distal end, as shown in FIGS. 3A-3B.

The channels 208-212 can be formed so as to maintain the desired relative positioning between the first positioning member 106, second spacing member 108, and lacerating member 110 during the leaflet cutting process as well as during the rotation of the positioning member 206. For example, each of the channels 208-212 can be angled with respect to a longitudinal axis of the catheter 202. In some embodiments, the distal opening of channel 208 for the second spacing member 108 is at a circumferential position on the positioning member 206 opposite to that of the distal opening of channel 210 for the first positioning member 106. The distal opening of channel 212 for the lacerating member 110 can be positioned adjacent to the distal opening of channel 210 for the first spacing member, or at a circumferential position on the positioning member 206 between openings of channels 208 and 210, as illustrated in FIGS. 3A-3B. In some embodiments, the positioning member 206 can further include another channel (not shown), for example, extending through a central portion of the positioning member 206, through which a guidewire can be extended.

In some embodiments, the tool 200 has an inner shaft 204 extending therein and attached at its distal end to the proximal end of the positioning member 206, as shown in FIG. 3A. The inner shaft 204 can be coaxial with the catheter 202 and moveable (e.g., rotatable) independently of catheter 202 (which can be referred to as an outer shaft of the tool in some embodiments). The members 106-110 can extend through a lumen of the inner shaft to the respective channels 208-210 of the positioning member. The attachment of the inner shaft 204 to the positioning member 206 thus allows the positioning member 206 to be rotated at the end of catheter 202 by rotation of the inner shaft 204 at its proximal end (e.g., by rotating the shaft 204 directly or via a handle at its proximal end). Alternatively, inner shaft 204 can be omitted in favor of attaching the positioning member 206 to the distal end of catheter 202, whereby rotation of the positioning member 206 with respect to the existing valvular structure is accomplished by rotation of the entire catheter 202 (e.g., by rotation the catheter 202 directly or via a handle at its proximal end). Since at least a portion of members 106-110 are retained within their respective channels 208-212, their positions relative to reach other are maintained throughout the rotational movement of the positioning member 206. Thus, the second tool 200 allows members 106-110 to be circumferentially repositioned to perform the cutting procedure on another leaflet 14 by simple rotation of only a single component, e.g., the positioning member 206, rather than requiring independent positioning of each of the members 106-110 as in the first tool 100.

In some instances, it may be desirable to cut one or more commissures 36 of the existing valvular structure, instead of or in addition to modification of the unattached portions (e.g., mid-region or free end) of the leaflets 14. By cutting the commissures 36, the leaflets 14 of the existing valvular structure may collapse distally (e.g., toward left ventricle 26). With the leaflets 14 in the collapsed orientation, obstruction of the coronary artery ostia, thereby allowing blood to flow toward the coronary arteries 22, 24 once the new prosthetic valve is installed within the existing valvular structure.

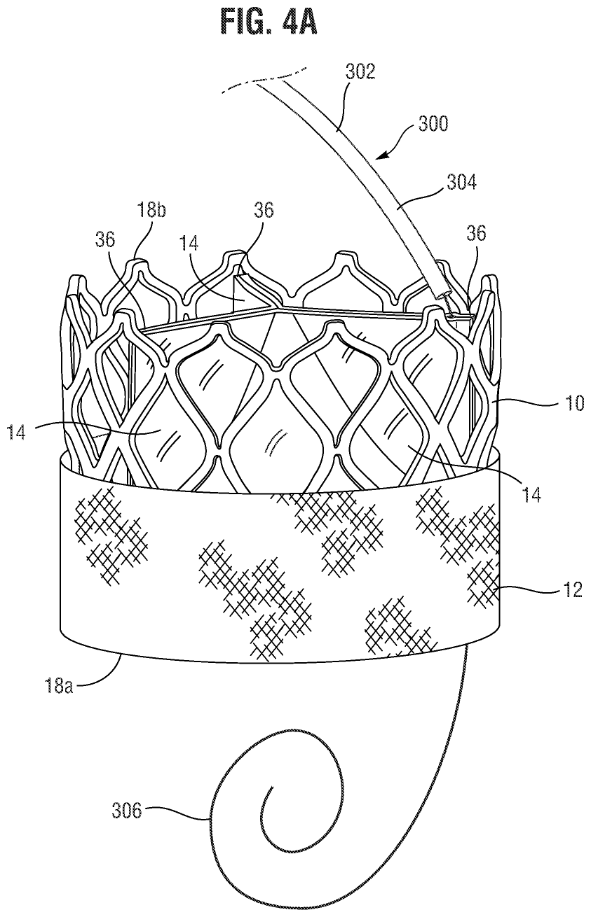

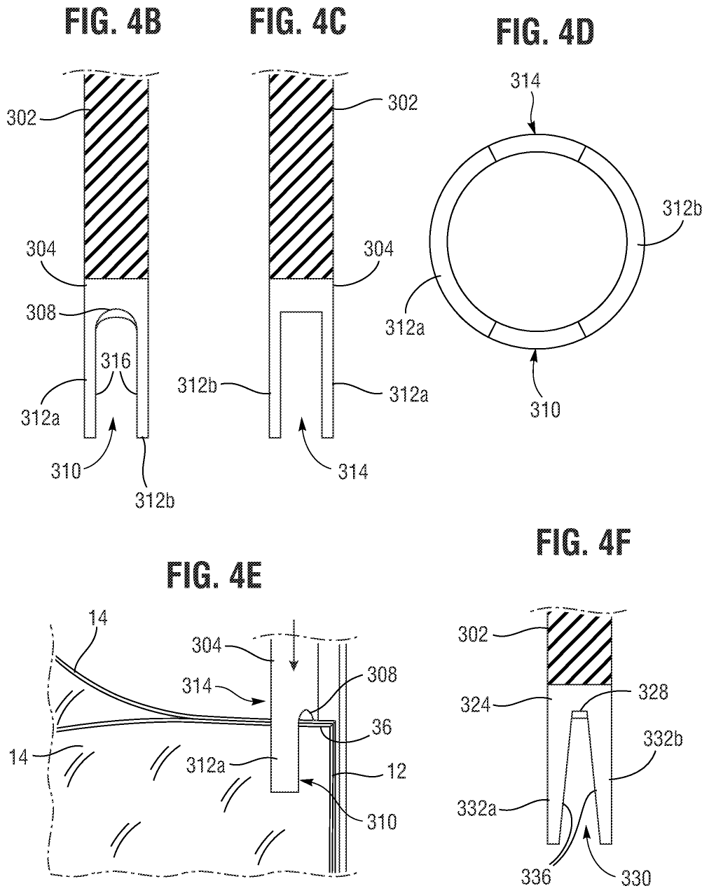

For example, FIGS. 4A-4E illustrate a third exemplary tool 300 that can be used to cut a commissure 36 of a valvular structure of an existing heart valve (whether a native aortic valve or a previously implanted prosthetic valve). The tool 300 comprises a catheter 302 (which also can be referred to as a shaft of the tool in some embodiments) with a distal end configured to be disposed within an ascending aorta of a subject. A substantially-cylindrical head portion 304 can be disposed at the distal end of the catheter 302. The head portion 304 can have structures that grip and cut the commissure 36. For example, the head portion 304 can have a first recess 310 that extends along an axial direction of the head portion, as shown in FIG. 4B. The first recess 310 can be defined by axially-extending edges 316 of first arm 312a and second arm 312b (e.g., portions of a circumferential wall of the head portion). The first recess 310 may be U-shaped with a curved proximal edge (e.g., as shown in FIG. 4B), have a substantially rectangular shape (e.g., similar to recess 314 in FIG. 4C), or have any other shape (e.g., an A-shape as in FIG. 4F, where head portion 324 has a recess 330 defined between edges 336 of arms 332a, 332b that narrows in the proximal direction to a cutting region 328). The opening at the distal end of recess 310 can be sized to accommodate the thickness of commissure 36 (e.g., double the thickness of a single leaflet) between the edges 316. At the proximal end of the first recess 310, the head portion 304 can include a mechanical cutting element 308, for example, a sharp edge or blade, which may be shaped to correspond to the shape of the recess (e.g., straight or arcuate). Alternatively or additionally, the cutting element 308 can be configured to cut tissue of the commissure 36 in contact therewith by application of electrical energy (e.g., RF energy). For example, cutting element 308 may be a non-insulated portion of the head portion 304.

The head portion 304 can have a structure that passively grips or abuts commissure 36 prior to or during cutting of the commissure 36 by the cutting element of the first recess 310. For example, the head portion 304 can have a second recess 314 on an opposite side of the head portion 304 from the first recess 310, as shown in FIGS. 4C-4D. The second recess 314 can also be defined by axially-extending edges of the first and second arms 312a, 312b. The second recess 314 can be sized and shaped similar to the first recess 310 or different from the first recess 310. For example, the second recess 314 may extend in the axial direction longer than the first recess 310. The second recess 314 may be U-shaped with a curved proximal edge (e.g., similar to recess 310 in FIG. 4B), have a substantially rectangular shape (e.g., as shown in FIG. 4C), or have any other shape (e.g., an A-shape as in FIG. 4F, where head portion 324 has a recess 330 defined between edges 336 of arms 332a, 332b that narrows in the proximal direction). The opening at the distal end of recessing 314 can be sized to accommodate the thickness of commissure 36 between the edges of arms 312a, 312b.

Alternatively or additionally, the head portion 304 can have a structure that actively grips commissure 36, for example, by a clamping mechanism.

Referring to FIGS. 4A and 4E, the third tool 300 is shown at various stages for cutting a commissure 36 of an existing valvular structure to avoid coronary artery obstruction by subsequent implantation of a prosthetic heart valve. Catheter shaft 302 can be advanced to the existing valvular structure (e.g., previously implanted valve 10 in FIG. 4A) from the ascending aorta. In some embodiments, a guidewire 306 can extend through a lumen of the catheter 302 toward the existing valve 10 to align the head portion 304 with the commissure 36 to be cut. In particular, the alignment can take advantage of the tendency of guidewire 306 to orient itself to a point of minimal energy, thereby displacing toward commissure 36 instead of a mid-region of leaflet 14. The commissure 36 thus acts as a receiving notch for the spring-like tensed guidewire 306. The guidewire 306 can extend through a center of the commissure 36 (e.g., between facing surfaces of adjacent leaflets 14).

The catheter 302 is then advanced over the guidewire 306 such that commissure 36 is received within recesses 310, 314 of the head portion 304, as shown in FIG. 4E. In some embodiments, the cutting recess 310 can be disposed on a radially outer side of head portion 304 facing a frame 12 of the valve 10 while the gripping recess 314 can be disposed on a radially inner side of head portion 304 facing a center of frame 12. In other embodiments, positions of the cutting and gripping recesses 310, 314 can be reversed, with the gripping recess 314 being between the cutting recess 310 and the frame 12 along the radial direction. In either case, as the head portion 304 advances in the distal direction, the proximal edge of the commissure 36 moves into contact with cutting element 308 of recess 310, which proceeds to slice through the commissure 36 using mechanical (e.g., sharp edge or blade) or electrical (e.g., RF energy applied to a non-insulated portion at the proximal end of recess 310) means.

If additional commissures 36 of the existing valvular structure are to be cut, the third tool 300, along with guidewire 306, can be retracted and repositioned with respect to an adjacent commissure. Cutting by the head portion 304 may then be repeated for the adjacent commissure in a manner similar to that described above. For example, when the existing valvular structure has three leaflets 14 and three commissures 36, the head portion 304 may be used to cut at least two of the three commissures, in order to allow the leaflets of the existing valvular structure to collapse distally out of the way of the coronary artery ostia.

As part of the ViV procedure, the new prosthetic heart valve in a crimped state can subsequently be advanced to the existing valvular structure with collapsed leaflets 14. The new valve is disposed within the valvular structure and expanded. However, the collapsed leaflets are disposed distal of the prosthetic heart valve, thereby allowing unobstructed blood flow from the outflow end of the new prosthetic valve to the coronary arteries 22, 24 once the ViV procedure is completed. Alternatively or additionally, when the existing valvular structure is a BAV, the collapsed leaflets can allow for improved mounting of the cylindrical prosthetic valve, which would otherwise be compromised by the non-circular geometry of the native BAV.

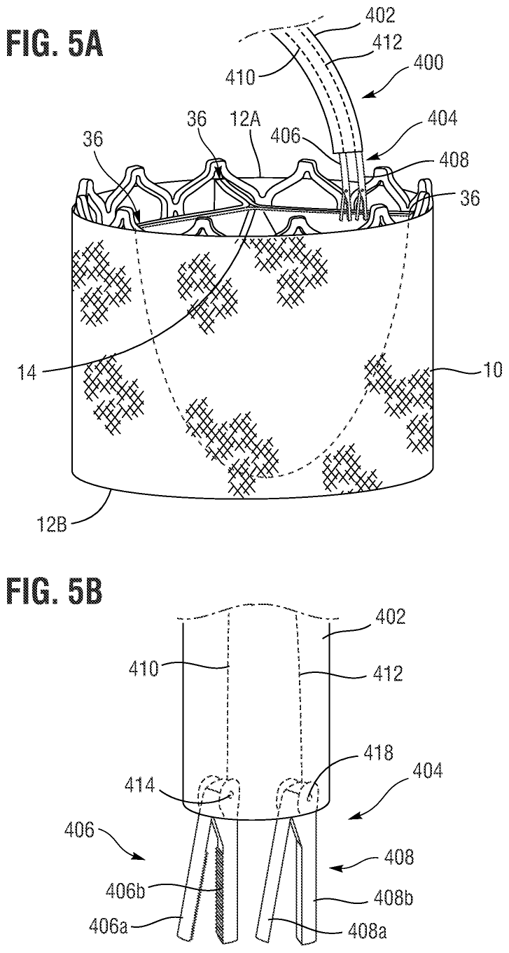

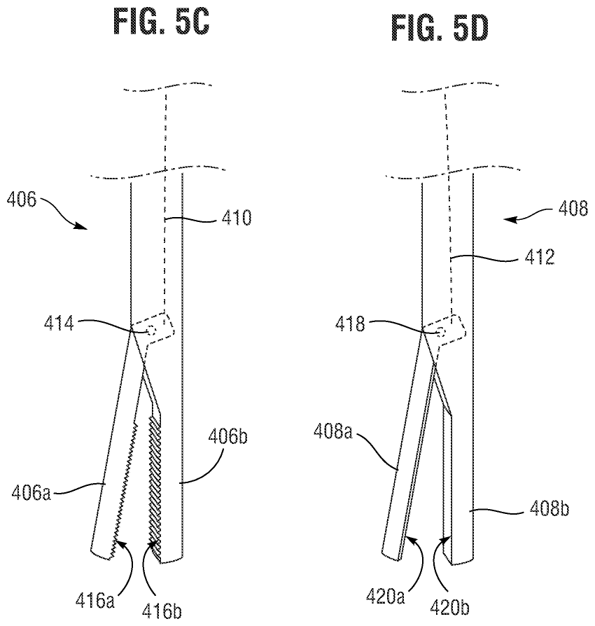

Instead of static recesses 310, 314 for cutting and passively gripping commissures 36, the head portion can be provided with active structures that move to cut and/or grip the commissures. For example, FIGS. 5A-5D illustrate a fourth exemplary tool 400 that can be used to cut a commissure 36 of a valvular structure of an existing heart valve (whether a native aortic valve or a previously implanted prosthetic valve 10). The tool 400 comprises a catheter 402 (which also can be referred to as a shaft of the tool in some embodiments) with a distal end configured to be disposed within an ascending aorta of a subject. A head portion 404 can be disposed at the distal end of the catheter 402. The head portion 404 can have active structures that grip and cut the commissure 36. For example, the head portion 404 can have a first active mechanism 406 for gripping and a second active mechanism 408 for cutting. A first member 410 (e.g., tether, suture, shaft, cable or wire) coupled to the first mechanism 406 can allow an operator to actuate the first mechanism 406, for example, via handle (not shown) at the proximal end of catheter 402. Similarly, a second member 412 (e.g., tether, suture, shaft, cable or wire) coupled to second mechanism 408 can allow the operator to actuate the second mechanism, for example, via the same or a different handle at the proximal end of catheter 402.

The first mechanism 406 can have a first arm 406a and a second arm 406b that are movable with respect to each other, as shown in FIGS. 5B-5C. For example, the first arm 406a and/or the second arm 406b can have respective facing edges that are enhanced for gripping commissure 36 (e.g., with serrated or toothed surfaces 416a, 416b). In some embodiments, the first arm 406a and second arm 406b are coupled together to allow pivoting or rotating motion relative to each other (e.g., similar to a scissor mechanism). In some embodiments, the second gripping arm 406b is held stationary (e.g., relative to catheter 402) while the first gripping arm 406a pivots around hinge 414 relative to the second gripping arm 406b. For example, as shown in FIG. 5C, the second gripping arm 406b can be an extension of or connected to the distal end of a shaft that extends through the catheter 402 and the first member 410 can extend through or alongside the shaft of the second gripping arm 406b. The first member 410, which extends through a lumen of the catheter 402 and is coupled to the first gripping arm 406a, can be used to actuate the first gripping arm 406a about hinge 414, such as by pulling the first member 410 proximally relative to the shaft of the second gripping arm 406b. Alternatively, the first gripping arm 406a is held stationary (e.g., relative to catheter 402) while the second gripping arm 406b pivots around a hinge relative to the first gripping arm 406a.

Alternatively or additionally, additional elements can be included within catheter 402 and/or in the head portion 404 to position the gripping arms 406a, 406b prior to, during, or after actuation by an operator. For example, a spring element (not shown) can be provided to urge the gripping arms 406a, 406b together in a normally-closed configuration or away from each other in a normally-open configuration. In another example, a protrusion or actuation stop may be provided between to limit motion of the gripping arms 406a, 406b toward each other, for example, to set a minimal gap between the arms 406a, 406b to maximize gripping without prematurely damaging the leaflets prior to cutting by the second mechanism 408.

The second mechanism 408 can have a first arm 408a and a second arm 408b that are movable with respect to each other, as shown in FIGS. 5B and 5D. For example, the first arm 408a and/or the second arm 408b can have respective facing edges that have mechanical cutting features (e.g. sharp edges or blade portions 420a, 420b). Alternatively, the first arm 408a and/or the second arm 408b can be configured to cut using electrical energy (e.g. RF energy) applied thereto (e.g., via non-insulated edge portions 420a, 420b). In some embodiments, the second member 412 or another member (e.g., cable or wire) extending through the lumen of catheter 402 may be configured to convey electrical energy (e.g., RF energy) to the first cutting arm 408a, the second cutting arm 408b, or portions thereof to effect cutting of commissure tissue.

In some embodiments, the first arm 408a and second arm 408b are coupled together to allow pivoting or rotating motion relative to each other (e.g., similar to a scissor mechanism). In some embodiments, the second cutting arm 408b is held stationary (e.g., relative to catheter 402) while the first cutting arm 408a pivots around hinge 418 relative to the second cutting arm 408b. For example, as shown in FIG. 5D, the second cutting arm 408b can be an extension of or connected to the distal end of a shaft that extends through the catheter 402 and the second member 412 can extend through or alongside the shaft of the second arm 408b. The second member 412, which extends through the lumen of the catheter 402 and is coupled to the first cutting arm 408a, can be used to actuate the first cutting arm 408a about hinge 418, such as by pulling the second member 412 proximally relative to the shaft of the second cutting arm 408b. Alternatively, the first cutting arm 408a is held stationary (e.g., relative to catheter 402) while the second cutting arm 408b pivots around a hinge relative to the first cutting arm 408a. Alternatively or additionally, additional elements can be included within catheter 402 and/or in the head portion 404 to position the cutting arms 408a, 408b prior to, during, or after actuation by an operator. For example, a spring element (not shown) can be provided to urge the cutting arms 408a, 408b together in a normally-closed configuration or away from each other in a normally-open configuration.

The operation of the fourth tool 400 to cut commissure 36 of an existing valvular structure may be similar to that of the third tool 300, except that the gripping and cutting are performed by active structures. In particular, catheter shaft 402 can be advanced to the existing valvular structure (e.g., previously implanted valve 10 in FIG. 5A) from the ascending aorta. In some embodiments, a guidewire (not shown) can extend through a lumen of the catheter 402 toward the existing valve 10 to align the head portion 404 with the commissure 36 to be cut. The alignment can take advantage of the tendency of the guidewire to orient itself to a point of minimal energy, thereby displacing toward a center of commissure 36 (e.g., between facing surfaces of adjacent leaflets 14) instead of a mid-region of leaflet 14. The commissure 36 thus acts as a receiving notch for the spring-like tensed guidewire.

The catheter 402 is then advanced over the guidewire such that commissure 36 is received within open recesses between gripping arms 406a, 406b and between cutting arms 408a, 408b, as shown in FIG. 5A. For example, the first gripping arm 406a can be pivoted away from the second gripping arm 406b, and the first cutting arm 408a can be pivoted away from the second cutting arm 408b. In some embodiments, the cutting mechanism 408 can be disposed on a radially outer side of head portion 404 facing a frame 12 of the valve 10 while the gripping mechanism 406 can be disposed on a radially inner side of head portion 404 facing a center of frame 12. In other embodiments, positions of the cutting and gripping mechanism 408, 406 can be reversed, with the gripping mechanism 406 being between the cutting mechanism 408 and the frame 12 along the radial direction.

Once the commissure 36 is sufficiently between the gripping and cutting mechanism 406, 408 (e.g., when the proximal end of the commissure 36 is adjacent to or abutting a proximal edge of the recess between cutting arms 408a, 408b), the commissure 36 can be gripped by the gripping mechanism 406, for example, by actuating first member 410 to cause the first gripping arm 406a to pivot towards the second gripping arm 406b, thereby clamping portions of the commissure 36 between surfaces 416a, 416b. After being gripped by mechanism 406, commissure 36 can then be cut by the cutting mechanism 408, for example, by actuating second member 412 to cause the first cutting arm 408a to pivot toward the second cutting arm 408b, thereby slicing (e.g., via mechanical or electrical cutting means) through portions of the commissure 36 between surfaces 420a, 420b.

In some embodiments, the length of arms 408a, 408b of cutting mechanism 408 along the axial direction of the catheter 402 is greater than that of arms 406a, 406b of gripping mechanism 406. The arms 406a, 406b may thus be dimensioned to grip a sufficient portion (e.g., a minimal length) of the commissure 36. The arms 408a, 408b may be dimensioned to extend beyond a corresponding dimension of the commissure 36 so as to allow complete cutting of the commissure by a single closing of the recess between arms 408a, 408b (e.g., a single scissoring motion of mechanism 408). Alternatively or additionally, the actuation of the second member 412 may be repeated, for example, to open and close the recess between cutting arms 408a, 408b repeatedly so as to scissor slice through the commissure 36. In some embodiments, the gripping mechanism 406 may clamp the commissure 36 between arms 406a, 406b until cutting by the cutting mechanism 408 is completed. The active gripping mechanism 406 can act to stabilize the commissure 36 during cutting thereof by the cutting mechanism 408. Moreover, the active gripping mechanism 406 can be used to retract a portion of the commissure 36 retained between the gripping arms 406a, 406b after cutting by the cutting mechanism 408.

If additional commissures 36 of the existing valvular structure are to be cut, the fourth tool 400, along with the guidewire, can be retracted and repositioned with respect to an adjacent commissure. Cutting by the head portion 404 may then be repeated for the adjacent commissure in a manner similar to that described above. For example, when the existing valvular structure has three leaflets 14 and three commissures 36, the head portion 404 may be used to cut at least two of the three commissures, in order to allow the leaflets of the existing valvular structure to collapse distally out of the way of the coronary artery ostia.

As part of the ViV procedure, the new prosthetic heart valve in a crimped state can subsequently be advanced to the existing valvular structure with collapsed leaflets 14. The new valve is disposed within the valvular structure and expanded. However, the collapsed leaflets are disposed distal of the prosthetic heart valve, thereby allowing unobstructed blood flow from the outflow end of the new prosthetic valve to the coronary arteries 22, 24 once the ViV procedure is completed. Alternatively or additionally, when the existing valvular structure is a BAV, the collapsed leaflets can allow for improved mounting of the cylindrical prosthetic valve, which would otherwise be compromised by the non-circular geometry of the native BAV.

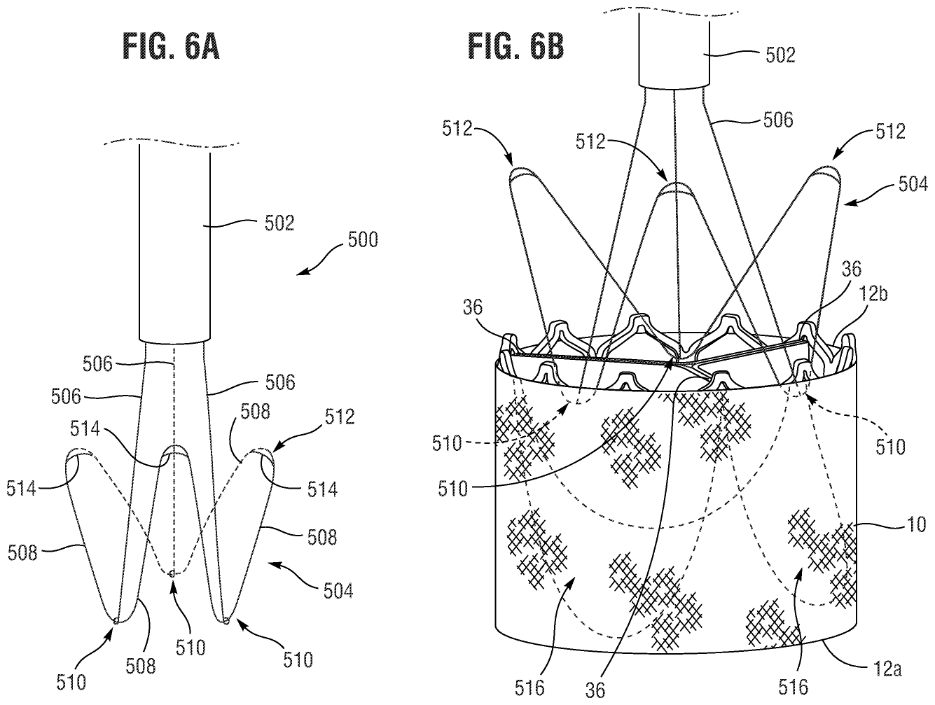

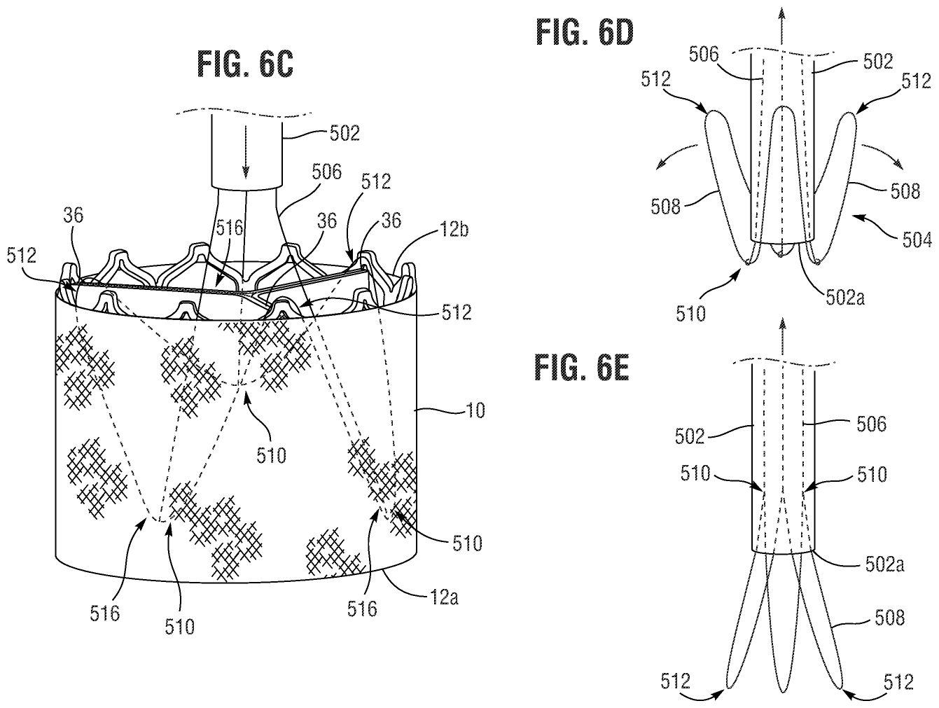

In some instances, it may be desirable to cut multiple commissures 36 at a same time, rather than sequentially cutting individual commissures as with the third 300 and fourth 400 tools. For example, FIG. 6A illustrates a fifth exemplary tool 500 that can be used to simultaneously cut through multiple commissures 36 of a valvular structure of an existing heart valve (whether a native aortic valve or a previously implanted prosthetic valve 10). The tool 500 comprises a catheter 502 (which can be referred to as a shaft of the tool in some embodiments) with a distal end configured to be disposed within an ascending aorta of a subject. The tool 500 can further include a three-dimensional-shaped flexible frame 504 that is configured to be expanded from a first size within catheter 502 to a second larger size outside catheter 502 (e.g., corresponding to a diameter where commissures 36 are located with respect to existing valvular structure). All or a portion of the frame 504 can be formed of a shape memory alloy, such as Nitinol, so as to automatically expand to the larger second size once released from the distal end of catheter 502. In this manner, the flexible frame 504 is self-expandable from a delivery state to its functional size once deployed from the catheter.

The flexible frame 504 can have a plurality of distal apices 510, a plurality of proximal apices 512, and a plurality of struts 508 that connect the distal and proximal apices together. In some embodiments, the frame 504 can be formed from a single wire, with bent sections of the wire forming apices 510, 512, and straight sections of the wire forming struts 508. Alternatively, multiple straight wires can be joined together, for example, at apices 510, 512. Each apex 510, 512 can have an arcuate shape (e.g., U-shape), a pointed shape (e.g., a V-shape), or any other shape (e.g., flat sections connected at opposite ends to corresponding angled struts 508). In some embodiments, the distal apices 510 can be disposed radially inward for the proximal apices 512 when the frame 504 is in the expanded second size.

Each proximal apex 512 can correspond to a respective one of the commissures 36 of the existing valvular structure. For example, a number of the proximal apices 512 of frame 504 can equal the number of commissures 36 of the existing valvular structure. Each proximal apex 512 can include a respective cutting element 514 configured to cut through a commissure 36 brought into contact therewith. For example, the cutting element 514 can be a sharp edge of the frame or a blade attached to the frame. Alternatively, the cutting element 514 can be configured to cut using electrical energy (e.g., RF energy) applied thereto. For example, the cutting element 514 can be a non-insulated metal portion of the frame or a separate member attached to the frame.

A plurality of support arms 506 can be coupled to the frame 504, for example, at distal apices 510. The support arms 506 can be used to position the frame 504 axially with respect to the catheter 502 (e.g., moving the frame 504 from within the catheter 502 to the existing valvular structure or vice versa) and/or circumferentially with respect to the valvular structure (e.g., to align distal apices 510 with corresponding recesses 516 formed between adjacent commissures 36). All or a portion of each support arm 506 can also be formed of a shape memory alloy, such as Nitinol.

Referring to FIGS. 6B-6C, the fifth tool 500 is shown at alignment and cutting stages, respectively, for simultaneously cutting multiple commissures 36 of an existing valvular structure to avoid coronary artery obstruction by subsequent implantation of a prosthetic heart valve. Catheter shaft 502 can be advanced to the existing valvular structure (e.g., previously implanted valve 10 in FIG. 6B) from the ascending aorta. During the advancing of catheter 502, the support arms 506 and frame 504 can be retained within a lumen of the catheter 502. Upon reaching the valve 10, the support arms 506 can be pushed distally, along with the frame 504 coupled thereto, out of the lumen of the catheter shaft 502. Alternatively or additionally, the support arms 506 and frame 504 can be maintained in place while the catheter shaft 502 can be retracted proximally to expose the frame 504 from the distal end of the catheter 502.

The frame 504 and/or support arms 506 can expand radially outwards, thereby increasing the diameter of the frame 504 such that locations of proximal apices 512 approach an inner diameter of valve frame 12, or at least match radial locations of the commissures 36 of valve 10. In some embodiments, the frame 504 may be rotatable about an axis of the catheter shaft 502, for example, to enable an operator to align distal apices 510 with corresponding pockets 516 between proximal surfaces of leaflets 14 and an inner wall of valve frame 12 and to align commissures 36 with the proximal apices 512. The shape and arrangement of distal apices 510 and/or struts 508 may also act to urge the frame 504 into alignment with commissures 36 as the frame 504 is advanced into contact with the valvular structure of existing valve 10.

Once the frame 504 is aligned with and contacting the existing valvular structure, it can be pushed further in the distal direction in order to simultaneously slice through multiple commissures 36 using the cutting elements 514 at the proximal apices 512, as shown in FIG. 6C. As noted above, the cutting elements 514 can cut the commissures 36 using mechanical (e.g., sharp edge or blade) or electrical (e.g., RF energy applied to a non-insulated portion at the proximal apex 512) means. The cutting of the multiple commissures can thus allow the leaflets 14 of the heart valve 10 to collapse distally out of the way of the coronary artery ostia prior to installation of the new prosthetic heart valve.

Referring to FIGS. 6D-6E, the fifth tool 500 is shown at first and second stages, respectively, for retraction of the frame 504 after cutting of the commissures 36 in preparation for removal from the subject. In the first stage of retraction, the frame 504 remains in the expanded state, for example, having a diameter at the proximal apices 512 that is greater than that of the catheter shaft 502, as shown in FIG. 6D. Support arms 506 can pull the distal apices 510 adjacent to the distal end of the catheter shaft 502. In the second stage of retraction, the support arms 506 are further retracted into the catheter 502, where interaction between walls of the catheter 502 and struts 508 of frame 504 cause deformation of the frame 504. For example, the struts 508 of frame 504 can be sufficiently flexible, such that they bend about distal apices 510. The struts 508 are thus pressed against and pushed by the distal lip of catheter 502, causing the struts 508 to bend until the proximal apices 512 are rotated to a position that is distal to the distal apices 512, as shown in FIG. 6E. The frame 504 can then be further retracted until it is fully retained within the catheter 502. The catheter 502 can then be removed from the subject and replaced with a delivery system for implant of the new prosthetic heart valve within the existing valvular structure.

As part of the ViV procedure, the new prosthetic heart valve in a crimped state can subsequently be advanced to the existing valvular structure with collapsed leaflets 14. The new valve is disposed within the valvular structure and expanded. However, the collapsed leaflets are disposed distal of the prosthetic heart valve, thereby allowing unobstructed blood flow from the outflow end of the new prosthetic valve to the coronary arteries 22, 24 once the ViV procedure is completed. Alternatively or additionally, when the existing valvular structure is a BAV, the collapsed leaflets can allow for improved mounting of the cylindrical prosthetic valve, which would otherwise be compromised by the non-circular geometry of the native BAV.

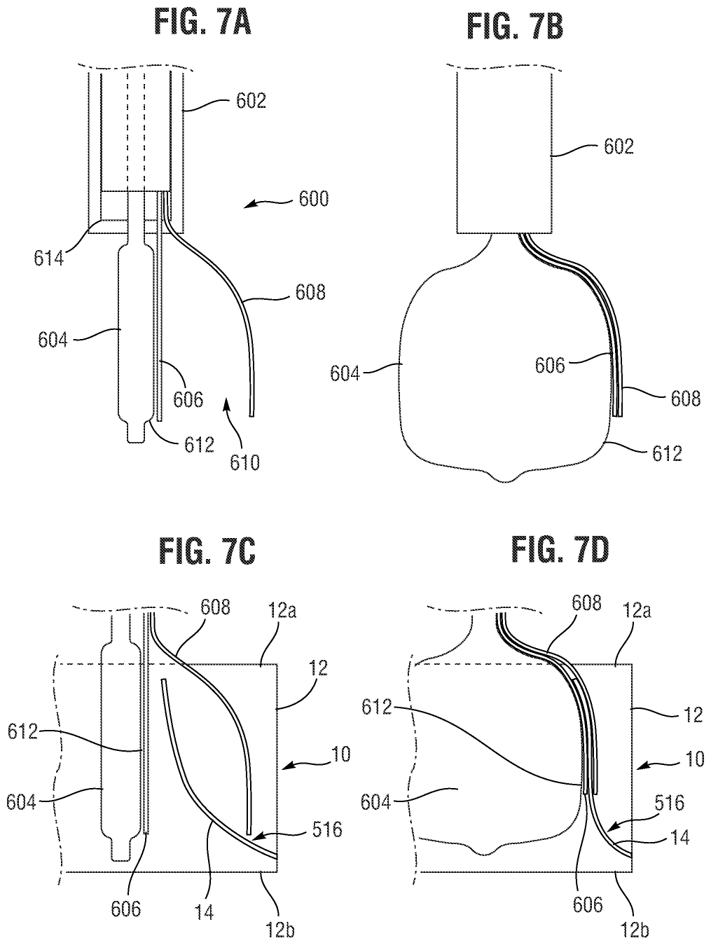

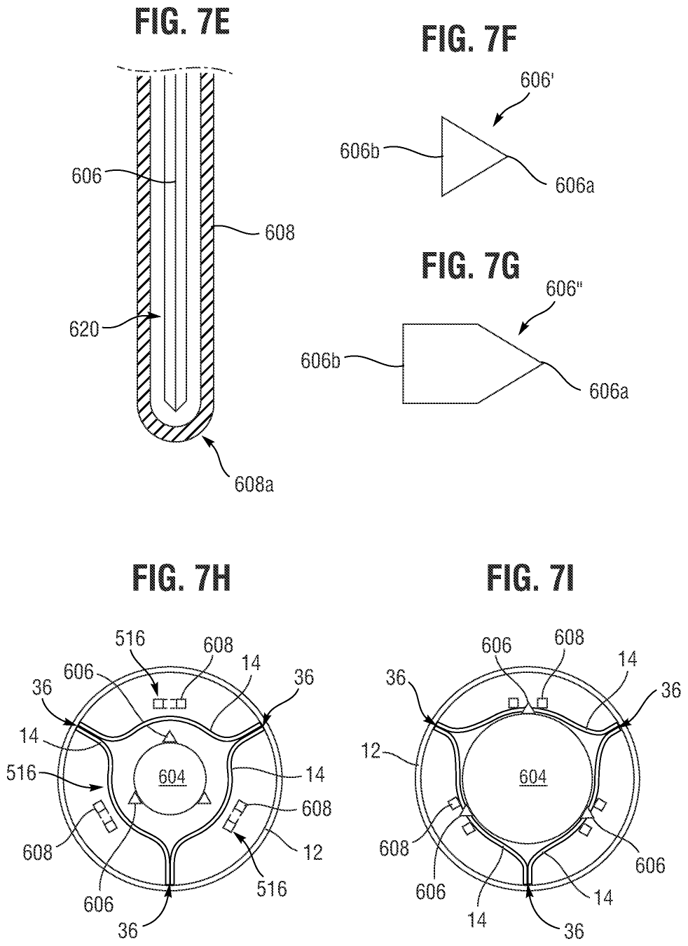

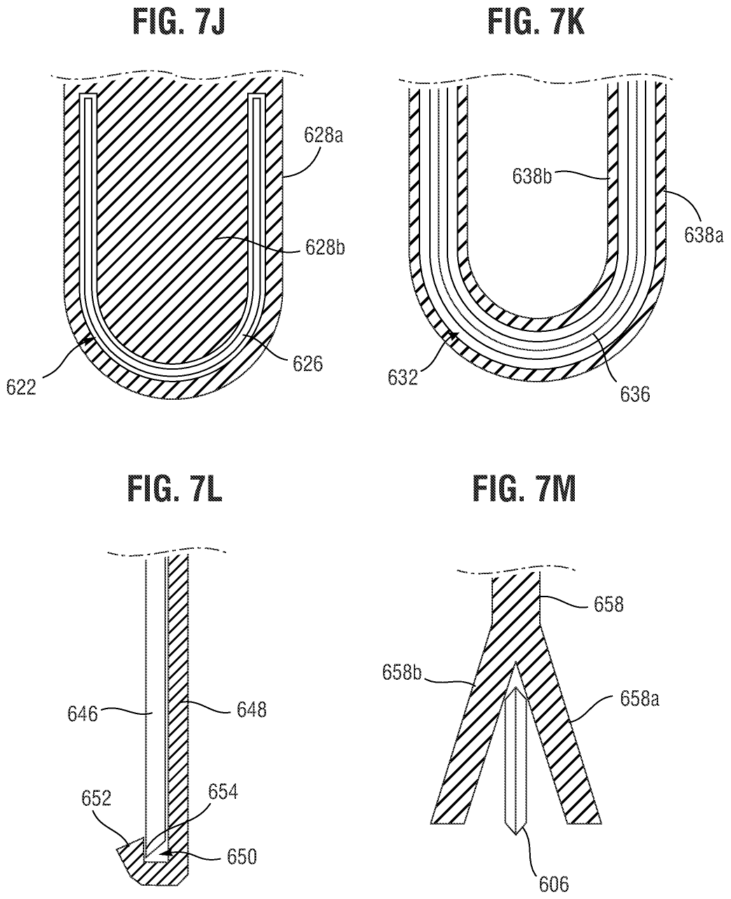

In some instances, it may be desirable to sequentially or simultaneously cut leaflets 14 of an existing valvular structure using a device that expands to grip and cut the leaflets. For example, FIGS. 7A-7E illustrate a sixth exemplary tool 600 that can be used to cut leaflets 14 of a valvular structure of an existing heart valve (whether a native aortic valve or a previously implanted prosthetic valve 10). The tool 600 comprises a catheter 602 (which can be referred to as a shaft of the tool in some embodiments) with a distal end configured to be disposed within an ascending aorta of a subject. The tool 600 can further include a positioning member 608, a cutting member 606, and an expansion device 604. In some embodiments, the tool 600 can further include a coupling member 614 that connects the positioning member 608, cutting member 606, and expansion device 604 together. Displacing the coupling member 614 distally along a lumen of the catheter 602 can thus simultaneously move the positioning member 608, cutting member 606, and expansion device 604 out of the distal end of the catheter 602.

For example, the expansion device can be a balloon 604 (shown deflated in FIG. 7A and inflated in FIG. 7B), a mechanically expandable frame (not shown) (which can be expanded by mechanical actuators), or a self-expanding frame (not shown) (e.g., formed of a shape memory alloy), or an annular braided structure (which can be self-expandable or expanded by mechanical actuators). The cutting member 606 can be disposed radially between the expansion device and the positioning member 608. By increasing the diameter of the expansion device (e.g., by inflating the balloon 604 as shown in FIG. 7B), surface 612 can displace cutting member 606 toward the positioning member 608 such that a leaflet 14 therebetween is initially gripped by interaction between cutting member 606 and positioning member 608, and then cut by cutting member 606.

The positioning member 608 can be configured as a prong that is bent or curved outward along its length. For example, the positioning member 608 can formed of a shape memory alloy, such as Nitinol, that automatically adopts the radially outward shape once fully extended from the distal end of catheter 602. The positioning member 608 can have an internal window 620, as shown in FIG. 7E. For example, the window 620 can have an inverted U-shape with an opening at a distal end of the positioning member 608, an inverted V-shape 658 (e.g., as shown in FIG. 7M) with an opening between flared arms 658a, 658b at a distal end of the prong, a rectangular shape with an open or closed distal end, a U-shape (e.g., as shown in FIG. 7E) with a closed distal end 608a, a V-shape with a closed distal end, a circular, oval, or elliptical shape with a closed distal end, or any other shape. In another example, the positioning member 608 can have a closed U-shaped window 622 (e.g., as shown in FIG. 7J) or V-shaped window (not shown) defined between outer portion 628a and inner portion 628b. In still another example, the positioning member 608 can have an outer member 638a separate from an inner member 638b to form a U-shaped channel 632 (e.g., as shown in FIG. 7K) or V-shaped channel (not shown) for the cutting element.

The cutting element 606 can be configured as a flexible member aligned with the positioning member 608, such that the cutting element 606 is displaced into the internal window 620 when the expansion device is expanded. In some embodiments, the cutting element 606 is formed as blade having a sharp edge that extends radially outward. Alternatively, the cutting element 606 is configured to cut using electrical energy (e.g., RF energy) applied thereto. For example, the cutting element can have a triangular cross-section 606′ as in FIG. 7F, a pentagonal cross-section 606″ as in FIG. 7G, or any other cross-section. For example, the cutting element has a pointed cutting edge 606a and a substantially flat surface 606b via which the expansion device 604 urges the cutting element into the corresponding window 620 of the positioning member 608 to effect cutting.

In some embodiments, the cutting element 606, or a portion thereof that interfaces with the positioning member 608, can have a shape complementary to that of the window 620 or channel of the positioning member 608. For example, the cutting element can have a U-shape 626 (e.g., as shown in FIG. 7J) or a V-shape that corresponds to the window 622 of the positioning member, or a U-shape 636 (e.g., as shown in FIG. 7K) or V-shape (not shown) that corresponds to channel 632 between adjacent portions (e.g., outer member 638a and inner member 638b) of a positioning member (or separate positioning members).

The U-shape or V-shape configurations of the positioning member 608 and/or cutting element 606 (e.g., as shown in FIGS. 7E, 7J, 7K, and 7M) can facilitate retraction thereof back into catheter 602 at the completion of a procedure. For example, the space between axially-extending portions of the U-shape or V-shape can accommodate flexure of the axially-extending portions toward each other, thereby allowing the positioning member 608 or cutting element 606 to contract to fit within the catheter 602.

Referring to FIGS. 7C-7D, the sixth tool 600 is shown at alignment and cutting stages, respectively, for cutting of leaflet 14 of an existing valvular structure to avoid coronary artery obstruction by subsequent implantation of a prosthetic heart valve. Catheter shaft 602 can be advanced to the existing valvular structure (e.g., previously implanted valve 10 in FIG. 7C) from the ascending aorta. During the advancing of catheter 602, the expansion device 604, the cutting element 606, and the positioning member 608 can be retained within a lumen of the catheter 602. Upon reaching the valve 10, the expansion device 604, the cutting element 606, and the positioning member 608 can be pushed distally out of the lumen of the catheter shaft 602. The positioning member 608 can extend radially outward once extended from the catheter 602 and tends to align with a pocket 516 formed between leaflet 14 and frame 12 (or an outer wall of the anatomy when the existing valve is the native aortic valve) when pushed distally toward the base of leaflet 14.

Meanwhile, the free end of leaflet 14 can be disposed in gap 610 between the positioning member 608 and the cutting element 606, as shown in FIG. 7C. When the expansion device 604 is expanded (e.g., by inflation of the balloon), the cutting element 606 is pushed radially outward by circumferential surface 612 into and/or against window 620 of the positioning member 608, thereby collapsing gap 610. The leaflet 14 is thus captured between the cutting element 606 and the positioning member 608, as shown in FIG. 7D, such that the cutting element 606 forms a cut in the leaflet 14 via mechanical means (e.g., sharp edge 606a) or electrical means (e.g., RF energy applied thereto). In some embodiments, the cutting element 606 forms a longitudinal cut in the leaflet 14 (e.g., to form a gash that splays the free end of the leaflet 14). In some embodiments, a shape of the cut in the leaflet corresponds to a shape of the cutting element and/or the positioning member, for example, a U-shaped or V-shaped cut.

If additional cutting of one or more leaflets 14 of the existing valvular structure is desired, for example, to provide an opening for both coronary arteries 22, 24, then the catheter 602 can be repositioned (e.g., rotated) with respect to the next leaflet after partial or full retraction of the expansion device 604, cutting element 606, and/or positioning member 608 into the catheter 602. The cutting of the next leaflet 14 can then be performed in a similar manner as described above for FIGS. 7C-7D. Alternatively, tool 600 can have a number of cutting elements 606 and positioning members 608 to correspond to a number of leaflets of the existing valvular structure. The expansion device 604 of tool 600 can actuate the cutting elements 606 to simultaneously cut multiple leaflets 14 of the existing valvular structure. For example, the tool 600 can have three cutting elements 606 and three corresponding positioning members 608 for simultaneously cut leaflets 14, as shown in FIGS. 7H-7I.

When no further cutting is desired, the expansion device 604, cutting element 606, and positioning member 608 can be fully retracted and the catheter 102 retrieved from the subject. As part of the ViV procedure, the new prosthetic heart valve in a crimped state can subsequently be advanced to the existing valvular structure with cut leaflets 14. The new valve is disposed within the valvular structure and expanded, such that the leaflets 14 are disposed on an external surface of the new valve frame. However, the one or more cuts formed in the leaflets 14 allows blood to flow from the outflow end of the new prosthetic valve to the coronary arteries 22, 24 once the ViV procedure is completed. Alternatively or additionally, when the existing valvular structure is a BAV, the cut leaflets can allow for improved mounting of the cylindrical prosthetic valve, which would otherwise be compromised by the non-circular geometry of the native BAV.

In some instances, the cutting element may be effective to fully sever a portion of the leaflet from the rest of the leaflet. In such cases, it may be desirable to remove the severed leaflet portion from the subject, for example, by using the cutting tool. For example, FIG. 7L illustrates an exemplary variation of the sixth tool 600 that can be used to cut the leaflets and secure the severed leaflet portion for removal with the tool. For example, the cutting member 646 and positioning member 648 can be constructed with a snap-fit feature. Thus, when the cutting member 646 is urged toward the positioning member 648 by balloon 604, the cutting member 646 engages with and is retained by the positioning member 648 (or vice versa). The portion of the leaflet disposed between the cutting member 646 and positioning member 648 is thus trapped and can be removed from the subject with the tool.

For example, the snap-fit feature can include a portion 652 of the positioning member 648 that protrudes radially inward to define a recess 650 into which a distal end of the cutting element 646 may fit. The positioning member portion 652 may have an angled surface that cooperates with a corresponding angled surface 654 of the cutting element 646. As the cutting element 646 is urged radially outward by the balloon 604, the surface 654 deflects positioning member portion 652 distally until the cutting element 646 moves into recess 650, after which the positioning member portion 652 returns to its original shape, thereby retaining the cutting element 646 to the positioning member 648 after balloon 604 is deflated. Other snap-fit configurations are also possible according to one or more contemplated embodiments.

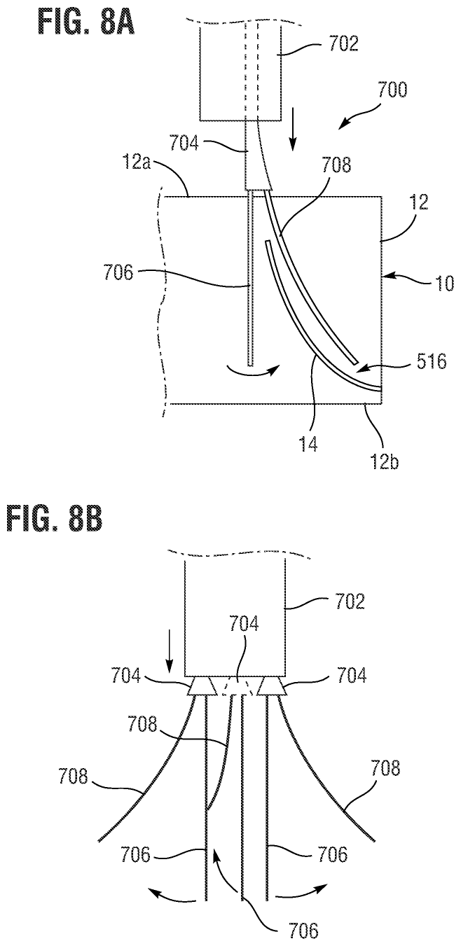

In some instances, other mechanisms can be used to urge the cutting element into contact with the positioning member instead of an expandable device. For example, FIG. 8A illustrates a seventh exemplary tool 700 that can be used to cut leaflets 14 of a valvular structure of an existing heart valve (whether a native aortic valve or a previously implanted prosthetic valve 10). Tool 700 comprises a catheter 702 (which can be referred to as a shaft of the tool in some embodiments) with a distal end configured to be disposed within an ascending aorta of a subject. The tool 700 can further include a positioning member 708, a cutting element 706, and a tubular member 704. The positioning member 708 and cutting element 706 can be disposed within tubular member 704 and can be axially displaced with respect to the tubular member 704 so as to extend from the distal end of the tubular member 704 in order to interact with the valvular structure.

The positioning member 708 can be configured as a prong that is bent or curved outward along its length. For example, the positioning member 708 can formed of a shape memory alloy, such as Nitinol, that automatically adopts the radially outward shape once fully extended from the distal end of tubular member 704. The cutting element 706 can be configured as a flexible member that is disposed radially inward of the positioning member 708 once fully extended from the distal end of tubular member 704. Thus, the exposed portions of the positioning member 708 and cutting element 706 can be positioned on opposite sides of a leaflet 14 of the valvular structure, as shown in FIG. 8A. The geometry of the tubular member 704 can be designed such that a predetermined amount of axial displacement of the tubular member 704 produces a predetermined reduction in spacing between the cutting element 706 and positioning member 708. For example, the tubular member 704 can be tapered along a portion of its length, with its distal end have a diameter greater than that of its proximal end. By distally moving the tubular member 704 along its longitudinal axis over the exposed portions of the cutting element 706 and positioning member 708, the geometry of the tubular member 704 forces the positioning member 708 and cutting element 706 toward each other in order to cut a portion of leaflet 14. In some embodiments, the cutting element 706 and positioning member 708 can then be moved proximally (e.g., by retracting catheter 702 together with components therein, by retracting tubular member 704 together with components therein into catheter 702, or by retracting the cutting element 706 and positioning member 708 into tubular member 704) to extend the initial cut in the leaflet 14.

If additional cutting of one or more leaflets 14 of the existing valvular structure is desired, for example, to provide an opening for both coronary arteries 22, 24, then the catheter 702 can be repositioned (e.g., rotated) with respect to the next leaflet after moving the tubular member 704 proximally to allow cutting element 706 and positioning member 708 to separate. The cutting of the next leaflet 14 can then be performed in a similar manner as described above. Alternatively, tool 700 can have a number of cutting elements 706 and positioning members 708 to correspond to a number of leaflets of the existing valvular structure. In some embodiments, the multiple cutting elements 706 and positioning members 708 can extend from a single tubular member 704, whereby moving the tubular member 704 distally simultaneously actuates each pair of cutting elements 706 and positioning members 708. Alternatively, each pair of cutting elements 706 and positioning members 708 may have their own respective tubular member 704, which may move together with or independently of other tubular members 704. For example, tubular members 704 of tool 700 can actuate the cutting elements 706 to simultaneously cut multiple leaflets 14 of the existing valvular structure. For example, the tool 700 can have three cutting elements 706, three positioning members 708, and three tubular members 704, as shown in FIG. 8B.

When no further cutting is desired, the tubular member 704, cutting element 706, and positioning member 708 can be fully retracted and the catheter 102 retrieved from the subject. The seventh tool 700 and variations thereof may thus be substantially similar to the sixth tool 600 and variations thereof described above with respect to FIGS. 7A-7M, except that the expansion device 604 is replaced with one or more tubular members 704. In a particular example, the positioning member 708 can have an inverted V-shape configuration with a recess open at the distal end, similar to that of positioning member in FIG. 7M, and the cutting element 706 can have a substantially linear configuration and be disposed between opposite arms of the positioning members, similar to that of cutting element 606 in FIG. 7M.

Such an inverted V-shape configuration 658 can accommodate retraction of the positioning member 708 into tubular member 704, for example, via flexing of opposing arms 658a, 658b toward each other.

As part of the ViV procedure, the new prosthetic heart valve in a crimped state can subsequently be advanced to the existing valvular structure with cut leaflets 14. The new valve is disposed within the valvular structure and expanded, such that the leaflets 14 are disposed on an external surface of the new valve frame. However, the one or more cuts formed in the leaflets 14 allows blood to flow from the outflow end of the new prosthetic valve to the coronary arteries 22, 24 once the ViV procedure is completed. Alternatively or additionally, when the existing valvular structure is a BAV, the cut leaflets can allow for improved mounting of the cylindrical prosthetic valve, which would otherwise be compromised by the non-circular geometry of the native BAV.

In some instances, the tool can employ a hook-shaped lacerating member for cutting a leaflet 14 of an existing valvular structure. The lacerating member can be initially positioned into a pocket formed between the leaflet and the valve frame (or vessel wall when applied to a native heart valve). A sharp tip of the hook-shaped lacerating member can be moved into contact with the facing leaflet, thereby piercing the leaflet. The lacerating member can then be moved proximally to form a gash extending from the point of initial piercing to the free end of the leaflet. The lacerating member may be formed of a shape memory alloy, such that it adopts the hook-shape only when deployed from a channel.

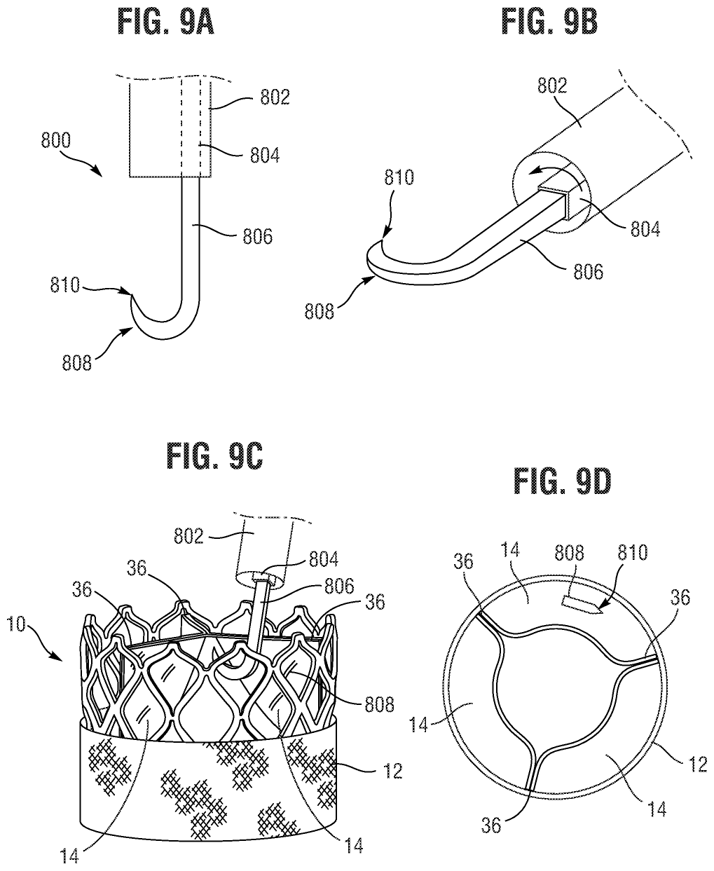

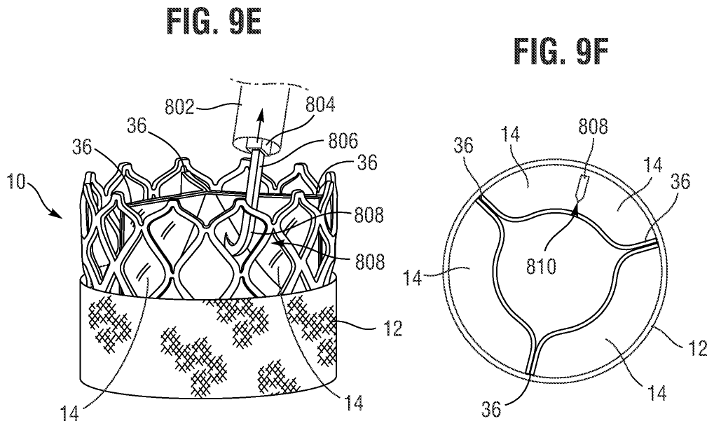

For example, FIG. 9A-9B illustrate an eighth exemplary tool 800, which uses a hook-shaped lacerating member to cut a leaflet of an existing valvular structure. The tool 800 comprises a catheter 802 (which can be referred to a shaft of the tool in some embodiments) with a distal end configured to be disposed within an ascending aorta of a subject. The catheter 802 has a rail or channel 804 extending therein. In some embodiments, the catheter 802 can be a multi-lumen catheter, and the channel 804 can be one of the multiple lumens thereof. The channel 804 can have a non-circular cross-section, such as rectangular, polygonal, oval, or elliptical. A lacerating member 806 can be disposed within the channel 804 and can have a non-circular cross-section, for example, a cross-section complementary to, but smaller than, that of the channel 804. The channel 804 can be rotatable within catheter 802 by an operator at its proximal end. The non-circular cross-sections of the channel 804 and the lacerating member 806 can allow rotation of the channel 804 to be directly transmitted to the lacerating member 806.

The lacerating member 806 is movable along the longitudinal axis of the channel 804 from a proximal position, where the lacerating member 806 is fully contained within channel 804, to a distal position, where the lacerating member 806 extends from the distal end of the channel 804, as shown in FIG. 9A-9B. For example, the lacerating member 806 can be formed of a shape memory alloy such as Nitinol. Thus, when the lacerating member 806 is within channel 804, it adopts a substantially linear configuration, thereby allowing the lacerating member 806 to move axially within channel 804. However, when the lacerating member 806 is extended from channel 804, the end portion 808 of the lacerating member adopts a hook-shape with a sharp tip 810, as shown in FIGS. 9A-9B. In some embodiments, the shape memory of the lacerating member 806 may further bias it radially outward once extended from the distal end of channel 804.

Extending the lacerating member 806 from the channel 804 and manipulating a position of catheter 802 and an orientation of channel 804 can allow an operator to position the sharp tip for cutting a leaflet of the existing valvular structure. For example, as shown in FIGS. 9C-9D, the lacerating member 806 can be extended from the channel 804 and positioned in a first orientation with the sharp tip 810 pointing along a circumferential direction of the valvular structure (e.g., substantially parallel to a wall of the valve frame or a wall of the blood vessel). The first orientation of the lacerating member 806 can help it to align with a pocket between a leaflet 14 and the valve frame 12 (or the aortic wall 30 when dealing with the native valve) as it is pushed distally out of channel 804 or catheter 802. For example, the lacerating member 806 can be moved distally until it contacts a portion of the leaflet where it attaches to the valve frame 12 or native heart valve annulus.

Once positioned at a desired location with respect to leaflet 14, the lacerating member 806 can be rotated to a second orientation via rotation of the channel 804. For example, the lacerating member 806 can be rotated by 90° such that the sharp tip 810 of the hook-shaped end portion 808 points along the radial direction of the valvular structure toward a center thereof, as shown in FIGS. 9E-9F. The sharp tip 810 may contact and pierce through the facing leaflet 14. The lacerating member 806 can then be retracted proximally, such that the initial piercing of the leaflet 14 is extended through a free end of the leaflet by tearing, thereby forming an extended gash that splays the leaflet into separate portions. Alternatively or additionally, the extended gash can be formed using electrical energy (e.g., RF energy) applied to the lacerating member 806 or a portion thereof (e.g., a non-insulated portion of hook-shaped end 808) as the lacerating member 806 is retracted proximally. For example, the proximal motion of the lacerating member 806 can be by retracting the lacerating member 806 into the channel 804 while holding the catheter 802 at a static position, retracting the catheter 802 in a proximal direction while maintaining a position of lacerating member 806 with respect to the distal end of the catheter 802, or any other combination of motions of lacerating member 806, channel 804, and catheter 802. In some embodiments, the proximal retraction of the lacerating member 806 into channel 804 can further act to move the hook-shaped end portion 808 radially inwards and towards the facing leaflet 14, thereby facilitating contact and cutting of the leaflet 14 during retraction.

If additional cutting of one or more leaflets 14 of the existing valvular structure is desired, for example, to provide an opening for both coronary arteries 22, 24, then the catheter 802 can be repositioned (e.g., rotated) with respect to the next leaflet after partial or full retraction of lacerating member 806 into channel 804. The cutting of the next leaflet 14 can then be performed in a similar manner as described above for FIGS. 9C-9F. Alternatively, tool 800 can have a number of lacerating members 806 and respective channels 804 in catheter 802 to correspond to a number of leaflets of the existing valvular structure. Each lacerating member 806 and channel 804 can be manipulated to simultaneously or sequentially cut multiple leaflets 14 of the existing valvular structure. For example, the tool 800 can have three lacerating members 806 and three corresponding channels 804 for simultaneously cutting three leaflets 14.

When no further cutting is desired, the lacerating member can be fully retracted into channel 804 and the catheter 802 retrieved from the subject. As part of the ViV procedure, the new prosthetic heart valve in a crimped state can subsequently be advanced to the existing valvular structure with cut leaflets 14. The new valve is disposed within the valvular structure and expanded, such that the leaflets 14 are disposed on an external surface of the new valve frame. However, the one or more gashes formed in the leaflets 14 allows blood to flow from the outflow end of the new prosthetic valve to the coronary arteries 22, 24 once the ViV procedure is completed. Alternatively or additionally, when the existing valvular structure is a BAV, the one or more gashes formed in the leaflets can allow for improved mounting of the cylindrical prosthetic valve, which would otherwise be compromised by the non-circular geometry of the native BAV.

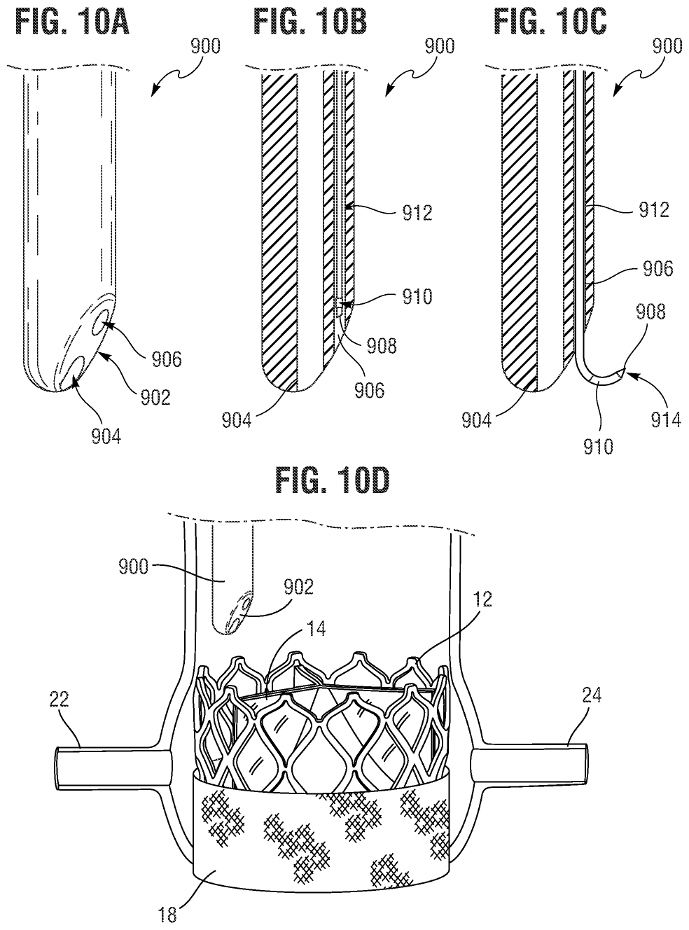

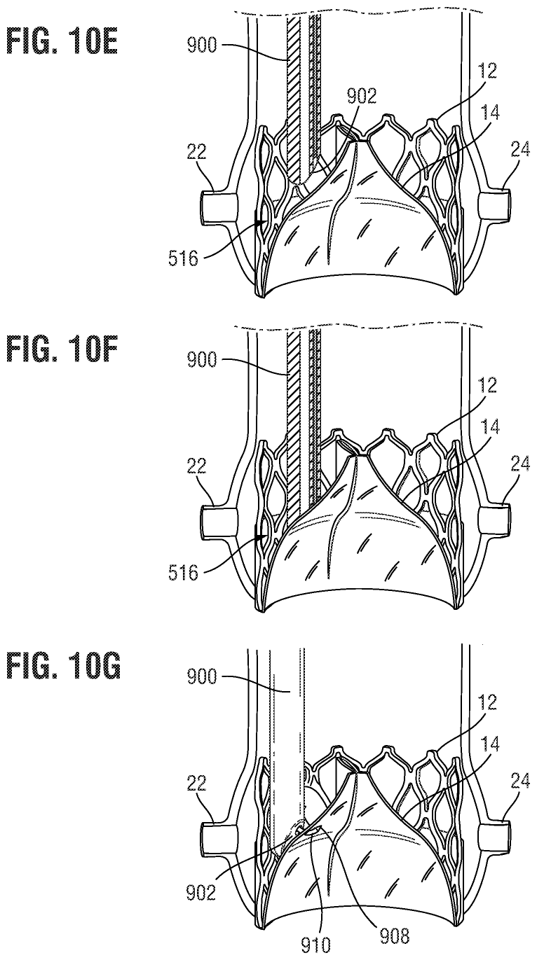

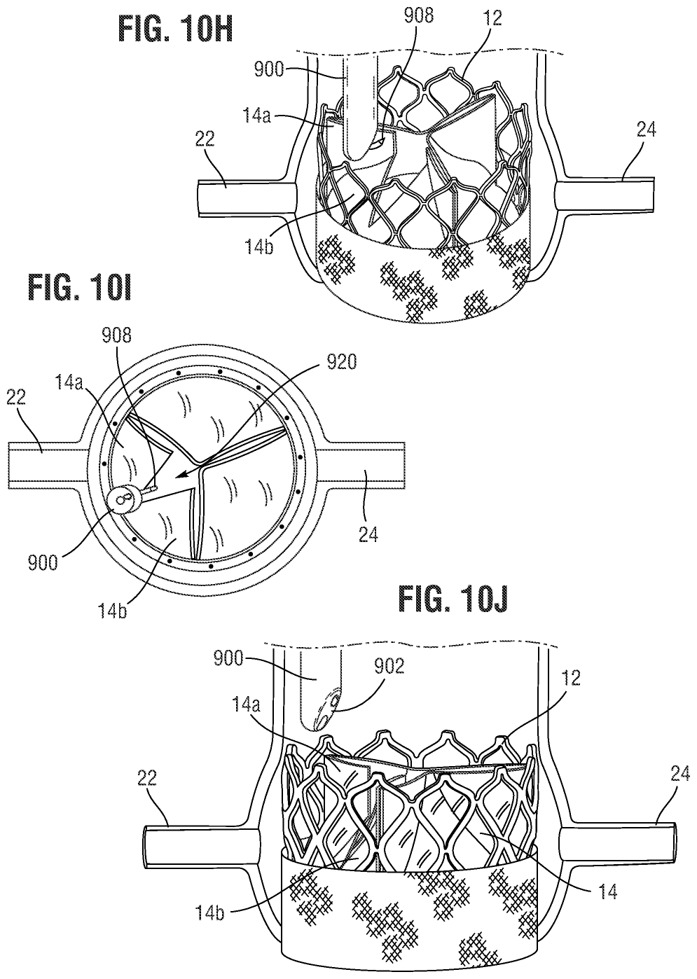

FIGS. 10A-10C illustrate a ninth exemplary tool 900, which uses a hook-shaped lacerating member to cut a leaflet of an existing valvular structure. The tool 900 comprises a delivery system or catheter with a distal end configured to be disposed within an ascending aorta of a subject. The delivery system can comprise a multi-lumen shaft having an angled surface 902 at its distal end with one or more openings therein. For example, the angled surface 902 can have a first opening or outlet corresponding to lumen 904, through which a guide wire (not shown) can extend. The angled surface 902 can also have a second opening or outlet corresponding to lumen 906, through which a lacerating member 907 can extend. The first opening corresponding to guide wire lumen 904 may be disposed on the angled surface 902 more distal than the second opening corresponding to lacerating member lumen 906.

The lacerating member 907 is movable along the longitudinal axis of the lumen 906 from a proximal position where the lacerating member 907 is fully contained within lumen 906 (as shown in FIG. 10B), to a distal position where an end portion 914 of the lacerating member 907 projects from the angled surface 902 (as shown in FIG. 10C). For example, the lacerating member 907 can be formed of a shape memory alloy such as Nitinol. Thus, when the lacerating member 907 is within lumen 906, it adopts a substantially linear configuration, thereby allowing the lacerating member 907 to move axially within lumen 906. However, when the lacerating member 907 is extended from lumen 906, the end portion 914 of the lacerating member 907 can adopt a hook-shape with a sharp tip 908, as shown in FIG. 10C.

In some embodiments, the lacerating member 907 is configured to cut a leaflet using electrical energy (e.g., RF energy) applied thereto. The end portion 914 of the lacerating member 907 can have a non-insulated section 910 via which tissue can be cut when the lacerating member is energized, for example, by application of appropriate electrical energy at a proximal end of the lacerating member. The lacerating member 907 can also have an insulated body portion 912. For example, the insulated body portion 912 may extend from the proximal end of the lacerating member 907 (e.g., where electrical energy is applied) to the non-insulated section 910, such that the delivery system is protected from the electrical energy application. In some embodiments, the section of end portion 914 from the sharp tip 908 to section 910 may also be insulated, such that cutting of tissue is restricted to a region spaced from both the angled surface 902 and the tip 908 (e.g., only where non-insulated section 910 is disposed).

Referring to FIGS. 10D-10J, various stages of a leaflet cutting process employing the ninth tool 900 are shown. As shown in FIG. 10D, the delivery system can be disposed within an ascending aorta of a subject. The delivery system can be aligned with a pocket 516 between a leaflet 14 and the valve frame 12 (or the aortic wall 30 when dealing with the native valve). The angled surface 902 of the delivery system may positioned to face toward a center of the valvular structure, for example, with the guide wire lumen 904 being disposed radially outward of the lacerating member lumen 906. In some embodiments, the delivery system may be aligned with a circumferential location of one of the coronary arteries 22, 24 (e.g., with the delivery system between the coronary artery 22 and a center of the valve frame 12 along a radial direction), as shown in FIG. 10E.

The delivery system can be advanced distally until the angled surface 902 contacts the facing leaflet 14, as shown in FIG. 10F. For example, the delivery system can be advanced such that distal edge of the angled surface 902 reaches a bottom of a suture line that connects the leaflet 14 to the valve frame 12. The angle of the surface 902 may follow the configuration of the leaflet 14, such that the leaflet contacts all, most, or at least a majority of the surface 902 once the delivery system is disposed within pocket 516. In some embodiments, a vacuum can be applied to one or both lumens 904, 906 of the tool to bring the leaflet into contact with the surface 902 and/or retain the leaflet against the surface 902. With the leaflet 14 in contact with angled surface 902, the lacerating member can be moved distally through lumen 906 so as to extend from the surface 902, as shown in FIG. 10G. The sharp tip 908 of the lacerating member end portion 914 thus pierces through the leaflet 14.

The lacerating member is moved distally until the end portion 914 is fully deployed from the angled surface 902, in which configuration the non-insulated section 910 aligns with the hole formed in the leaflet 14. Electrical energy can then be applied to the lacerating member (e.g., by applying RF energy at its proximal energy). Since body portion 912 and the portion at tip 908 are otherwise insulated, the application of electrical energy is effective only at non-insulated section 910 to cut tissue of the leaflet 14 in contact therewith. The delivery system and/or the lacerating member can then be moved proximally in order to further cut the leaflet 14 using electrical energy, as shown in FIGS. 10H-10I. For example, the tool 900 can be retracted proximally such that the initial piercing of the leaflet by sharp tip 908 is extended through a free end of the leaflet by electrical cutting, thereby forming an extended gash 920 that splays the leaflet into separate portions 14a, 14b. In some embodiments, the gash 920 may extend from at or near the leaflet suture line (e.g., offset by a distance corresponding to a distance between the distal-most end of the delivery system and the opening of lacerating member lumen 906) all the way to the free edge of the leaflet 14.

If additional cutting of one or more leaflets 14 of the existing valvular structure is desired, for example, to provide an opening for both coronary arteries 22, 24, then the tool 900 can be repositioned (e.g., rotated) with respect to the next leaflet after partial or full retraction of the lacerating member into lumen 906. The cutting of the next leaflet 14 can then be performed in a similar manner as described above for FIGS. 10D-10I. Alternatively, tool 900 can have a number of lacerating members and delivery systems to correspond to a number of leaflets of the existing valvular structure. For example, tool 900 can have multiple prongs spaced from each other along the circumferential direction and disposed to align with a respective one of the leaflets of the valvular structure. Each prong can be similarly constructed to the delivery system with lacerating member illustrated in FIGS. 10A-10C and may connect to a common central positioning member (e.g., catheter or mother delivery system). For example, the common central positioning member may have the guide wire lumen 904 to be shared for all prongs rather than each prong having its own guide wire lumen. The lacerating members in each delivery system prong can be manipulated to simultaneously or sequentially cut multiple leaflets 14 of the existing valvular structure. For example, the tool 900 can have three lacerating members and three corresponding delivery system prongs for simultaneously cutting three leaflets 14.

When no further cutting is desired, the lacerating member can be fully retracted into lumen 904, as shown in FIG. 10J. The tool 900 can then be retrieved from the subject. The gash 920 in the leaflet may create acute aortic insufficiency (AI) in the existing valvular structure since it will allow some of the blood to flow back through the gash 920 into the left ventricle during diastole. In some embodiments, in order to minimize the time of the AI, the leaflet cutting procedure using tool 900 can be conducted while another delivery system with a new prosthetic valve is already positioned in the aorta, with its guidewire resting in the left ventricle. The new prosthetic valve can then be deployed immediately as the tool 900 is removed.

As part of the ViV procedure, the new prosthetic heart valve in a crimped state can subsequently be advanced to the existing valvular structure with cut leaflets 14. The new valve is disposed within the valvular structure and expanded, such that the leaflets 14 are disposed on an external surface of the new valve frame. However, the one or more gashes formed in the leaflets 14 allows blood to flow from the outflow end of the new prosthetic valve to the coronary arteries 22, 24 once the ViV procedure is completed. Moreover, the implantation of the new valve may act to push outward and stretch the existing leaflets, which stretching may act to further expand the gash 920, thereby further improving access to the coronary arteries. Alternatively or additionally, when the existing valvular structure is a BAV, the one or more gashes formed in the leaflets can allow for improved mounting of the cylindrical prosthetic valve, which would otherwise be compromised by the non-circular geometry of the native BAV.

In some instances, the leaflet may be pierced, torn, or otherwise altered without forming a longitudinally-extending cut or gash. For example, a tool can have a tissue engagement member that extends from a catheter to pierce through a proximal surface of one or more leaflets of an existing valvular structure. The tissue engagement member can be in the form of a cable, wire, shaft, tube, or any structure. One or more sharp tips of the tissue engagement member can penetrate through the leaflet. In some embodiments, the tool can further have a cutting portion, for example, a cutting edge of the catheter that is used to sever the portion of the leaflet penetrated by the tissue engagement member to create a hole therein. In such cases, it may be desirable to remove a severed portion of the leaflet from the valvular structure, for example, to minimize debris that may adversely affect the subject or subsequent prosthetic heart valve implantation. Alternatively or additionally, the tissue engagement member can be retracted through the leaflet to cause tearing in a portion of the leaflet. Since any portion of the leaflet may be engaged by the tissue engagement member, high accuracy positioning of the tool with respect to the leaflet can be avoided.

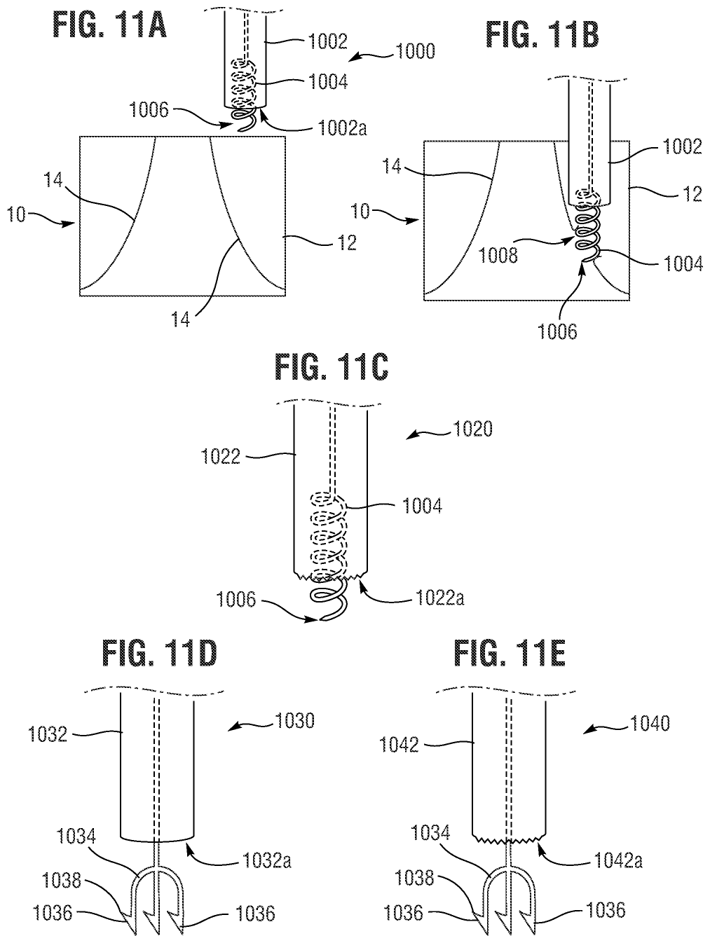

For example, FIGS. 11A-11B illustrate a tenth exemplary tool 1000, which employs a leaflet retaining device for piercing the leaflet of an existing valvular structure. The tool 1000 comprises a catheter 1002 (or other delivery shaft) with a distal end 1002a configured to be disposed within an ascending aorta of a subject. A leaflet retaining device 1004 can be disposed within the catheter 1002 and can be movable along the longitudinal axis of the catheter 1002 from a proximal position, where the leaflet retaining device 1004 does not extend beyond distal edge 1002a, to a distal position, where at least tip 1006 of leaflet retaining device 1004 extends beyond distal edge 1002a (as shown in FIG. 11A). The leaflet retaining device 1004 can also be configured to rotate about its longitudinal axis separate from and within catheter 1002.

The leaflet retaining device 1004 can have a cork-screw helical configuration with a sharp tip 1006 at its distal end. For example, the leaflet retaining device 1004 can be formed of a wire or cable of sufficient rigidity to transmit torque applied at its proximal end to its distal end, in particular, the sharp tip 1006. Extending the leaflet retaining device 1004 from the catheter 1002 into contact with the leaflet and rotating the leaflet retaining device 1004 can allow the leaflet retaining device 1004 to pierce and engage with the leaflet 14. Rotation of the leaflet retaining device 1004 can be effected by operator manipulation of an appropriate control mechanism at its proximal end, for example, turning of a knob.

For example, an operator can position the catheter distal end 1002a with respect to a leaflet 14 of the existing valvular structure. As shown in FIG. 11A, the leaflet retaining device 1004 may be extended distally toward the leaflet 14 from the catheter 1002 such that at least sharp tip 1006 is exposed. Alternatively, the leaflet retaining device 1004 may remain within catheter 1002 until catheter distal end 1002a is closer to or in contact with the leaflet 14. Then, the sharp tip 1006 of the leaflet retaining device 1004 can be brought into contact with the leaflet 14. Rotating the leaflet retaining device 1004 about its axis can screw the tip 1006 into and through the leaflet 14, as shown in FIG. 11B.

The leaflet retaining device 1004 can then be retracted proximally, where the coils of the helical shape can pull on portions of the leaflet in contact therewith to cause further tearing of the leaflet and widening of the aperture 1008 created by the initial piercing. The proximal motion of the leaflet retaining device 1004 can be by retracting the device 1004 into catheter 1002 while holding the catheter 1002 at a static position, retracting the catheter 1002 in a proximal direction while maintaining a position of leaflet retaining device 1004 with respect to the distal end 1002a of the catheter 1002, moving the catheter 1002 in a distal direction while holding the leaflet retaining device 1004 in a static position, or any other combination of motions of leaflet retaining device 1004 and catheter 1002.

Referring to FIG. 11D, an alternative configuration for a leaflet retaining device for use in a variation of the tenth tool is shown. Tool 1030 comprises a catheter 1032 (or other delivery shaft) with a distal end 1032a configured to be disposed within an ascending aorta of a subject. A leaflet retaining device 1034 can be disposed within the catheter 1032 and can be movable along the longitudinal axis of the catheter 1032 from a proximal position, where the leaflet retaining device 1034 does not extend beyond distal edge 1032a, to a distal position, where at least piercing tips 1036 of leaflet retaining device 1034 extend beyond distal edge 1032a (as shown in FIG. 11D). In some embodiments, the leaflet retaining device 1034 can also be configured to rotate about its longitudinal axis separate from and within catheter 1032.

The leaflet retaining device 1034 can have a multi-pronged pitch-fork configuration with a plurality of barbed piercing tips 1036 at its distal end. One, some, or all of the piercing tips 1036 may include barbed features that resist withdrawal of the respective tip 1036 once it has pierced the leaflet tissue. For example, each piercing tip 1036 can have an inverted prong section 1038. Extending the leaflet retaining device 1034 from the catheter 1032 into contact with the leaflet and can allow the tips 1036 to pierce and engage with the leaflet 14. Although FIG. 11D shows three tips 1036, additional or fewer tips are also possible. Moreover, although tips 1036 are shown in a linear planar arrangement in FIG. 11D, the tips can be oriented in a non-planar or three-dimensional arrangement (e.g., cross arrangement, circular arrangement, rectangular arrangement, etc.) in some embodiments.

An operator can position the catheter distal end 1032a with respect to a leaflet 14 of the existing valvular structure. The leaflet retaining device 1034 may be extended distally toward the leaflet 14 from the catheter 1032 such that at least tips 1036 are exposed. Alternatively, the leaflet retaining device 1034 may remain within catheter 1032 until catheter distal end 1032a is closer to or in contact with the leaflet 14. Then, the sharp tips 1036 of the leaflet retaining device 1034 can be brought into contact with the leaflet 14 and pierce through the leaflet 14. The leaflet retaining device 1034 can then be retracted proximally, where the inverted prong sections 1038 can pull on portions of the leaflet in contact therewith to cause further tearing of the leaflet. The proximal motion of the leaflet retaining device 1034 can be by retracting the device 1034 into catheter 1032 while holding the catheter 1032 at a static position, retracting the catheter 1032 in a proximal direction while maintaining a position of leaflet retaining device 1034 with respect to the distal end 1032a of the catheter 1032, moving the catheter 1032 in a distal direction while holding the leaflet retaining device 1034 in a static position, or any other combination of motions of leaflet retaining device 1034 and catheter 1032.

In some embodiments, the catheter or delivery shaft can also be configured to cut the leaflet 14 held by the leaflet retaining device. For example, the distal edge of the catheter can be a sharp edge, a serrated edge, or both. By contacting the distal edge with the leaflet, a portion of the leaflet can be severed from the rest of the leaflet. The severed portion of the leaflet can be held by the leaflet retaining device and retracted into the catheter with the leaflet retaining device for removal from the subject. For example, FIGS. 11C and 11E show alternative configurations for the catheter for use in variations of the tenth tool. Tool 1020 in FIG. 11C is similar to tool 1000 of FIGS. 11A-11B, except that catheter 1022 includes a serrated distal edge 1022a that can be used to cut a portion of a leaflet brought into contact therewith. Tool 1040 in FIG. 11E is similar to tool 1030 of FIG. 11D except that catheter 1042 includes a serrated distal edge 1042a that can be used to cut a portion of a leaflet brought into contact therewith.

For example, once a leaflet 14 is penetrated and grasped by leaflet retaining device 1004 or 1034, the leaflet 14 may be pulled into contact with the corresponding catheter distal edge 1022a or 1042a, for example, by retracting the leaflet retaining device into the catheter, by pushing the catheter toward the leaflet retaining device, or both. The contact with the corresponding catheter distal edge 1022a or 1042a may be sufficient to cut through the tissue and to sever a portion of the leaflet from the remainder. Alternatively or additionally, the catheter can be rotated about its longitudinal axis while keeping the leaflet retaining device stationary in order to saw or drill a circular hole within the leaflet. In such cases, the catheter can be configured to transmit torque along its length, and the rotation can be effected by operator manipulation of an appropriate control mechanism at its proximal end, such as by turning a knob. The severed portion may be defined by a circumference of the catheter distal edge. Since the severed portion remains engaged by the leaflet retaining device, it can be retracted into the catheter with the leaflet retaining device for removal from the subject.

If additional cutting of the same or different leaflets 14 of the existing valvular structure is desired, for example, to provide additional holes in the same leaflet or to provide holes for both coronary arteries 22, 24, then the tenth tool or variations thereof can be repositioned (e.g., laterally moving the catheter) after partial or full retraction of the leaflet retaining member into the catheter. The cutting of the next leaflet 14 can then be performed in a similar manner as described above for FIGS. 11A-11E. When no further cutting is desired, the leaflet retaining member, including any severed leaflet portions, can be fully retracted into the catheter. The tool can then be retrieved from the subject. As part of the ViV procedure, the new prosthetic heart valve in a crimped state can subsequently be advanced to the existing valvular structure with modified leaflets 14. The new valve is disposed within the valvular structure and expanded, such that the leaflets 14 are disposed on an external surface of the new valve frame. However, the one or more holes formed in the leaflets 14 allow blood to flow from the outflow end of the new prosthetic valve to the coronary arteries 22, 24 once the ViV procedure is completed.

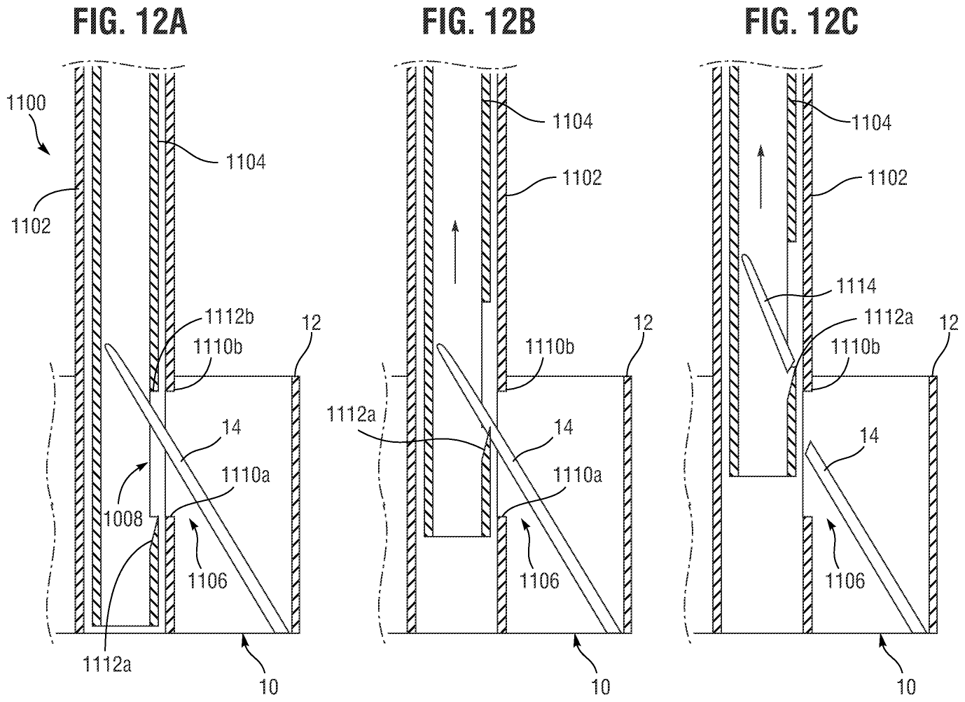

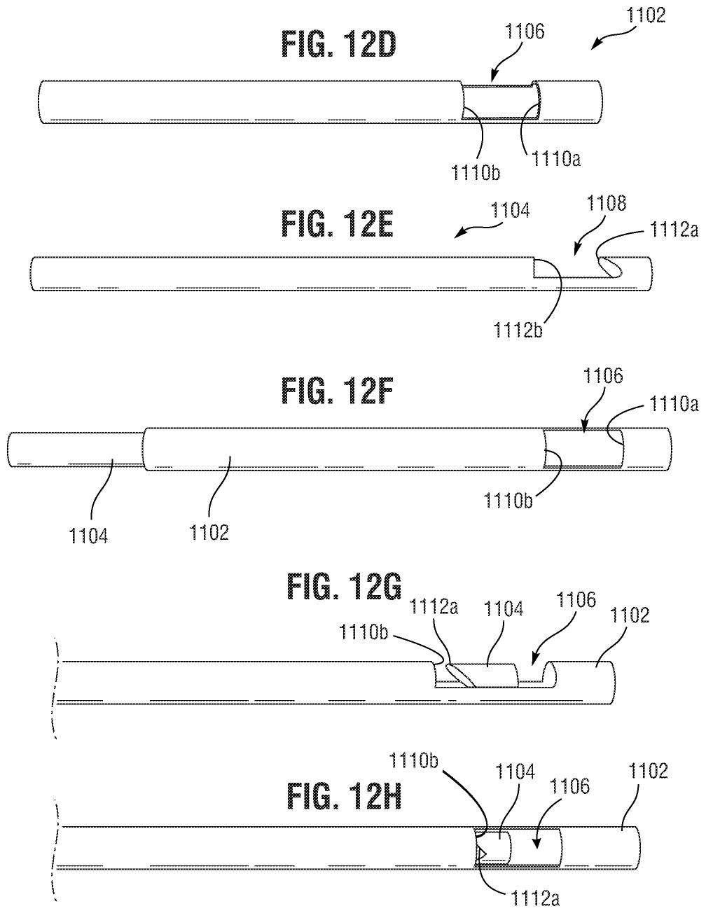

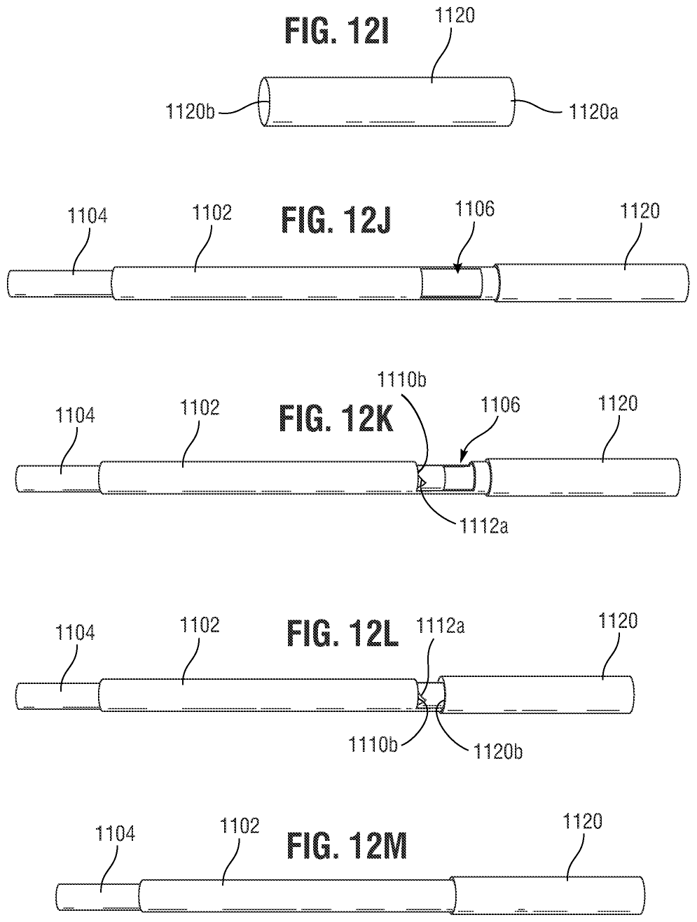

FIGS. 12A-12H illustrate an eleventh exemplary tool 1100, which is configured to cut and remove a portion of the leaflet of an existing valvular structure. The tool 1100 comprises an outer hollow shaft 1102 and an inner hollow shaft 1104. The inner shaft 1104 can be disposed within and movable relative to the outer shaft 1102 along an axial direction thereof. For example, the inner and outer shafts can have substantially-cylindrical cross-sections, and an outer diameter of the inner shaft 1104 is less than an inner diameter of the outer shaft 1102. A circumferential surface of the outer shaft 1102 can have a first window 1106. The first window 1106 can have a distal edge 1110a, a proximal edge 1110b separated from the distal edge 1110a along the axial direction, and a pair of axial edges extending between the distal edge 1110a and proximal edge 1110b, as shown in FIG. 12D. Similarly, a circumferential surface of the inner shaft 1104 can have a second window 1108. The second window 1108 can have a distal edge 1112a, a proximal edge 1112b separated from the distal edge 1112a along the axial direction, and a pair of axial edges extending between the distal edge 1112a and proximal edge 1112b, as shown in FIGS. 12E-12F.

At least the distal edge 1112a of the second window 1108 can be configured as a gripping edge, for example, a tooth that projects toward the proximal end of the inner shaft 1104. Alternatively or additionally, the distal edge 1112a may have a sharp edge or blade configured to cut tissue of the leaflet when sufficient pressure is applied (e.g., when the leaflet is clamped between the distal edge 1112a of the second window 1108 and the proximal edge 1110b of the first window 1106). Alternatively or additionally, the proximal edge 1110b of the first window 1106 can have a sharp edge or blade configured to cut tissue of the leaflet when sufficient pressure is applied thereto (e.g., when the leaflet is clamped between the distal edge 1112a of the second window 1108 and the proximal edge 1110b of the first window 1106).

The first window 1106 and the second window 1108 can be disposed close to distal ends of the outer shaft 1102 and inner shaft 1104 respectively. The distal end of the outer shaft 1102 and/or the distal end of the inner shaft 1104 may be closed, for example, to prevent ingress or egress of material between the subject and internal volumes of the shafts except via respective windows 1106, 1108. The distal end of the outer shaft 1102 (and the inner shaft 1104 contained therein) can be configured to be disposed within an ascending aorta of a subject and positioned with respect to a leaflet 14 of an existing valvular structure (e.g., previously implanted heart valve 10). En route to the existing valvular structure, the inner shaft 1104 can be disposed within the outer shaft 1106 such that inner window 1108 and outer window 1106 are misaligned, such that a circumferential outer surface of inner shaft 1104 blocks off window 1106. When the outer shaft 1106 reaches the existing valvular structure, the inner shaft 1104 can be rotated and/or moved axially within the outer shaft 1102 such that inner window 1108 and outer window 1106 are aligned, as shown in FIG. 12F. For example, the inner shaft 1104 can be positioned such that the distal edge 1112a of the inner window 1108 having the gripping edge (e.g., tooth) is not exposed by the outer window 1106 (e.g., distal edge 1110a of outer window 1106 is between the distal edge 1112a of the inner window 1108 and the proximal edge 1110b of outer window 1106 along the axial direction).

In this state, the tool 1100 can be advanced toward a free end or edge of leaflet 14, such that a portion of the leaflet 14 extends through the aligned windows 1106, 1108 into an internal volume of the inner shaft 1104, as shown in FIG. 12A. The inner shaft 1104 can then be retracted in the proximal direction relative to the outer shaft 1102, exposing the distal edge 1112a of the inner window 1108. Retraction of the inner shaft 1104 with respect to the outer shaft 1102 can be accomplished by holding the outer shaft 1102 in a fixed position and pulling the inner shaft 1104 in the proximal direction, holding the inner shaft 1104 in a fixed position and pushing the outer shaft 1102 in the distal direction, or a combination thereof.

Further retraction of the inner shaft 1104 in the proximal direction relative to the outer shaft 1102 brings the distal edge 1112a into contact with an underside of the leaflet 14, as shown in FIGS. 12B (with leaflet) and 12G (without leaflet), and can push or pull the leaflet into contact with proximal edge 1110b of the outer window 1106. The contact between the distal edge 1112a and the proximal edge 1110b can serve to clamp the leaflet between the inner shaft 1104 and the outer shaft 1106 for cutting. In particular, further retraction of the inner shaft 1104 in the proximal direction causes the distal edge 1112a to slice through the clamped portion of the leaflet, thereby severing a portion 1114 therefrom, as shown in FIGS. 12C (with leaflet) and 12H (without leaflet). As noted above, the proximal edge 1110b may also be configured to cut, such that further retraction of the inner shaft 1104 in the proximal direction causes the proximal edge 1110b to separately or additionally slice through the clamped portion of the leaflet. In some embodiments, the severed portion 1114 of the leaflet is retained within the internal volume of the inner shaft 1104 and can be removed with the tool 1100 from the subject or otherwise extracted (e.g., by vacuum applied to the internal volume of inner shaft 1104 when in the configuration of FIG. 12H).

In some instances, the distal edge 1112a of the inner window 1108 and/or the proximal edge 1110b of the outer window 1106 may be insufficient to cut the leaflet 14. Rather, the interaction between distal edge 1112a and proximal edge 1110b may only serve to clamp the leaflet 14 therebetween. Accordingly, in some embodiments, a separate cutting member can be provided to cut the leaflet once it is clamped by interaction between the window edges of the inner shaft 1104 and outer shaft 1102.

For example, FIGS. 12I-12M show a variation of the eleventh tool employing a separate cutting member. The cutting member 1120 can be configured as another hollow shaft that slides over the outer shaft 1102. Thus, the inner diameter of the cutting member 1120 is greater than an outer diameter of the outer shaft 1102. The cutting member 1120 can have a distal circumferential edge 1120a and a proximal circumferential edge 1120b. In some embodiments, the proximal edge 1120b, or at least a portion thereof, has a sharp edge or blade (e.g., a guillotine-style angled sharp blade or a series of serrated teeth) configured to cut tissue of the leaflet, and the opposite distal edge 1120a is configured as an atraumatic edge (e.g., a blunt, rounded, or smoothed edge designed to avoid cutting or injury to the subject). In such a configuration, the cutting member 1120 can be disposed distally of outer window 1106 and can be pulled proximally with respect to the outer shaft 1102 to cut a portion of leaflet 14 held between the distal end 1112a of the inner window 1108 and the proximal end 1110b of the outer window 1106. In other embodiments, the distal edge 1120a, or at least a portion thereof, has a sharp edge or blade (e.g., a guillotine-style angled sharp blade or a series of serrated teeth) configured to cut tissue of the leaflet, and the opposite proximal edge 1120b is configured as an atraumatic edge. In such a configuration, the cutting member 1120 can be disposed proximally of outer window 1106 and can be pushed distally with respect to the outer shaft 1102 to cut a portion of leaflet 14 held between the distal edge 1112a of the inner window 1108 and the proximal end 1110b of the outer window 1106.

In either configuration, the cutting member 1120 can be moved either proximally or distally along the axial direction by an appropriate axial member. For example, a positioning member, such as a rod, flat bar, or curved bar, can be coupled to a portion of the proximal edge 1120b of the cutting member 1120. The positioning member may thus be disposed adjacent to or follow an external circumferential surface of the outer shaft 1102. The positioning member may be disposed on a side of the outer shaft 1102 opposite to that of window 1106 so as not to obstruct window 1106 during the leaflet cutting procedure. Alternatively, the positioning member can be coupled to the distal end of the cutting member 1120. In such a configuration, the distal ends of the outer shaft 1102 and the inner shaft 1104 may be open, or at least have an opening for the positioning member to pass therethrough. The distal end of the cutting member may be closed, for example, to prevent ingress or egress of material between the subject and internal volumes of the shafts except via respective windows 1106, 1108. The positioning member may thus extend through an internal volume of the inner shaft 1104 and through an internal volume of the cutting member 1120 to attach to the distal end of the cutting member, for example, at a center thereof. By pushing or pulling on the positioning member, the cutting member 1120 can be moved along the axial direction. In such a configuration, the axial distance between the proximal edge 1120b and the proximal end 1110b of the outer window 1106 when the cutting member 1120 is in its initial position should be less than the axial distance between the distal end of the outer shaft 1102 and the distal end of the cutting member 1120 in the initial position, so as to allow for sufficient axial travel to provide cutting by cutting member 1120 (e.g., before further proximal motion is prevented by distal end of the cutting member 1120 abutting the distal end of outer shaft 1102).

Operation of the tool with cutting member 1120 may be similar to that discussed above with respect to FIGS. 12A-12C, but with the addition of axially displacing the cutting member 1120 after clamping of the leaflet to provide cutting. For example, FIGS. 12J-12K correspond to FIGS. 12A-12B, but with cutting element 1120 disposed distally of outer window 1106. Once the leaflet is clamped by the configuration of FIG. 12K, the cutting member 1120 can be retracted proximally, as shown in FIG. 12L, such that sharp proximal edge 1120b of the cutting member 1120 contacts through tissue of the leaflet clamped between distal edge 1112a and proximal edge 1110b. Further retraction, as shown in FIG. 12M, can force the sharp proximal edge 1120b through the tissue, thereby cutting the leaflet.

Other arrangements and configurations for the cutting member are also possible according to one or more contemplated embodiments. For example, the cutting member can be disposed within the inner shaft 1104 (e.g., the cutting member has an outer diameter less than an inner diameter of the inner shaft 1104). In another example, the cutting member can be disposed between the inner shaft 1104 and the outer shaft 1102 (e.g., the cutting member has an outer diameter less than an inner diameter of the outer shaft 1102 and an inner diameter greater than an outer diameter of the inner shaft 1104). The cutting member can be moved axially with respect to the inner shaft 1104 to cut a portion of the leaflet clamped between edges of windows 1106, 1108. However, in these examples, the clamping afforded by interaction between the inner shaft 1104 and outer shaft 1102 may be less than optimal for cutting by the cutting member as compared to the illustrated examples.

If additional cutting of the same or different leaflets 14 of the existing valvular structure is desired, for example, to cut additional portions of the same leaflet or to cut portions of other leaflets for both coronary arteries 22, 24, then the eleventh tool or variations thereof can be repositioned (e.g., laterally moving the outer shaft). The cutting of the next leaflet 14 can then be performed in a similar manner as described above for FIGS. 12A-12M. For example, it may be desirable to provide a cut in the leaflet of at least 5 mm in the leaflet. However, the dimensions of the windows of tool 1100 may afford a cut of only approximately 2 mm. Accordingly, the tool 1100 may be repositioned with respect to the same leaflet to extend the cut, for example, by repeating the cutting process 2-3 times in order to achieve the desired minimum cut length.

When no further cutting is desired, the tool can be retrieved from the subject. As part of the ViV procedure, the new prosthetic heart valve in a crimped state can subsequently be advanced to the existing valvular structure with modified leaflets 14. The new valve is disposed within the valvular structure and expanded, such that the leaflets 14 are disposed on an external surface of the new valve frame. However, the cut leaflets 14 can allow blood to flow from the outflow end of the new prosthetic valve to the coronary arteries 22, 24 once the ViV procedure is completed. Alternatively or additionally, when the existing valvular structure is a BAV, the cut leaflets can allow for improved mounting of the cylindrical prosthetic valve, which would otherwise be compromised by the non-circular geometry of the native BAV.

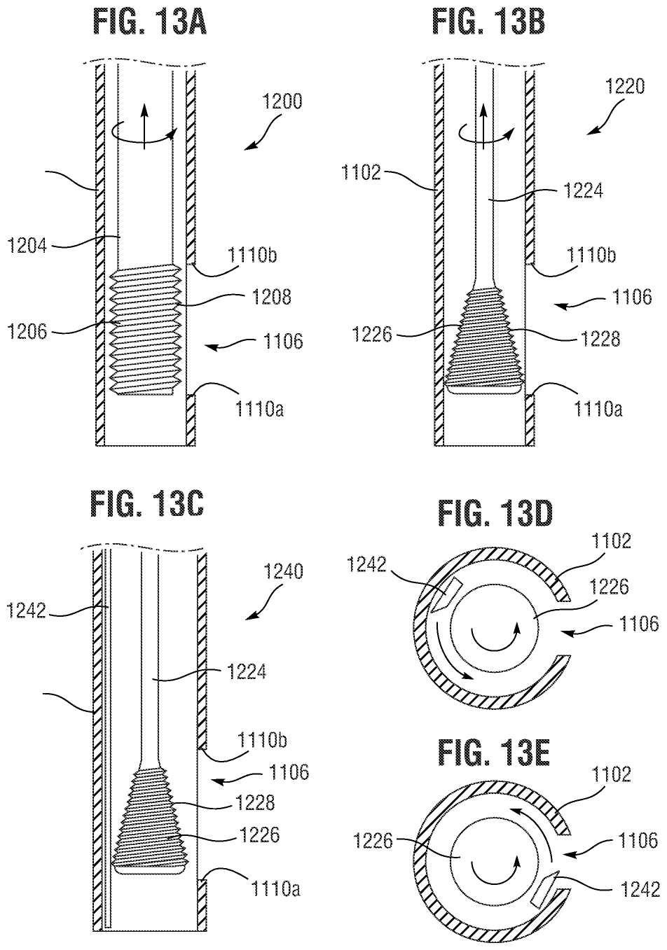

FIG. 13A illustrates a twelfth exemplary tool 1200 that is configured to cut a portion of a leaflet of an existing valvular structure. The tool 1200 comprises an outer hollow shaft 1102 similar to that employed in the eleventh tool 1100. Thus, the circumferential surface of the outer shaft 1102 has a first window 1106, which has a distal edge 1110a, a proximal edge 1110b separated from the distal edge 1110a along the axial direction, and a pair of axially-extending edges extending between the distal edge 1110a and proximal edge 1110b. The distal end of the outer shaft 1102 may be closed, for example, to prevent ingress or egress of material between the subject and internal volume of the shaft except via window 1106. However, instead of a hollow inner shaft as in the eleventh tool 1100, the twelfth tool 1200 comprises an inner shaft 1204 with a threaded portion 1206, which may be at or spaced from a distal end of the inner shaft 1204. The inner shaft 1204 is disposed within and movable relative to the outer shaft 1102 along an axial direction thereof.

The threaded portion 1206 can have an outer diameter that is less than an inner diameter of the outer shaft 1102. The outer diameter of the threaded portion 1206 may be substantially constant over its axial length. Alternatively, the outer diameter of the threaded portion 1206 may be variable, for example, taper from its distal end toward its proximal end, as shown in FIG. 13B. In some embodiments, the diameter of the inner shaft 1204 away from the threaded portion 1026 may be less than the threaded portion 1206.

The distal end of the outer shaft 1102 (and the inner shaft 1204 contained therein) can be configured to be disposed within an ascending aorta of a subject and positioned with respect to a leaflet 14 of an existing valvular structure (e.g., previously implanted heart valve 10). The inner shaft 1204 can be disposed such that threaded portion 1206 and outer window 1106 are aligned, as shown in FIG. 13A, or at least overlap. For example, the inner shaft 1204 can be positioned such that a proximal end of the threaded portion 1206 is adjacent to or near proximal edge 1110b of the outer window 1106. In this state, the tool 1200 can be advanced toward a free end or edge of leaflet (not shown), such that a portion of the leaflet extends through the window 1106 into contact with threads 1208 of threaded portion 1206. The inner shaft 1204 can then be retracted in the proximal direction relative to the outer shaft 1102. Retraction of the inner shaft 1204 with respect to the outer shaft 1102 can be accomplished by holding the outer shaft 1102 in a fixed axial position and pulling the inner shaft 1204 in the proximal direction, holding the inner shaft 1204 in a fixed axial position and pushing the outer shaft 1102 in the distal direction, or a combination thereof. At a same time, the inner shaft 1204 can be rotated about its axis, whereby interaction between threads 1208 may serve to pull the leaflet further into the window 1106.

In some embodiments, the proximal edge 1110b of the first window 1106 can have a sharp edge or blade configured to cut tissue of the leaflet when sufficient pressure is applied thereto (e.g., when the leaflet is clamped between the threads 1208 of the threaded portion 1206 and the proximal edge 1110b of the first window 1106). Thus, the combined rotational motion and retraction of inner shaft 1204 can increase the size of the leaflet portion cut by proximal edge 1110b. Additional or alternatively, the threads 1208 of portion 1206 can be configured as sharp threads, thereby providing further cutting of the leaflet in contact therewith, for example, a portion of the leaflet clamped between the proximal end threads of portion 1206 and the proximal edge 1110b or an inner circumferential surface of the outer shaft 1102. Where the threaded portion 1226 is tapered, as with variation 1220 shown in FIG. 13B, the threads 1228 can be arranged like an Archimedes screw such that gradually wider portions of the leaflet are cut as the inner shaft 1224 is further rotated and retracted in the proximal direction.

In some instances, the threads of the threaded portion of the inner shaft may not be able to provide sufficient cutting of the leaflet. Rather, the threads may merely cooperate with the outer shaft to clamp the leaflet therebetween (e.g., between threads 1228 and proximal edge 1110b of window 1106). Accordingly, in some embodiments, a separate cutting member can be provided to cut the leaflet once it is clamped. For example, FIGS. 13C-13E show a variation of the twelfth tool employing a separate cutting member. The cutting member 1242 can configured as an axially-extending blade (e.g., a gallstone-type angled blade) disposed between the inner shaft 1224 and the outer shaft 1102 along a radial direction. The cutting member 1242 can be movable around a circumference of the inner shaft 1224, in particular, the threaded portion 1226. For example, the cutting member 1242 can be positioned at an initial stowed location away from window 1106 (e.g., on a side of threaded portion 1226 opposite window 1106), as shown in FIGS. 13C-13D. Once the leaflet is clamped by the threaded portion 1226, the cutting member 1242 can be moved across window 1106 (e.g., by rotating the cutting member 1242 about a center of the outer shaft 1102), as shown in FIG. 13E, to cut through leaflet tissue within window 1106.

Other arrangements and configurations for the cutting member are also possible according to one or more contemplated embodiments. For example, the cutting member can be disposed over the outer shaft 1102 in a manner similar to cutting member 1120 in FIGS. 12I-12J. In another example, the cutting member can be disposed over the outer shaft 1102 and have a second window aligned with window 1106 of the outer shaft 1102. The second window of the cutting member can have an axial edge that is sharp or has a blade. The cutting member can thus be rotated with respect to the outer shaft 1102 in order to cut a portion of the leaflet within window 1106 clamped by the threaded portion 1228 using the axial edge of the second window.

If additional cutting of the same or different leaflets of the existing valvular structure is desired, for example, to cut additional portions of the same leaflet or to cut portions of other leaflets for both coronary arteries 22, 24, then the twelfth tool or variations thereof can be repositioned (e.g., laterally moving the outer shaft). The cutting of the next leaflet can then be performed in a similar manner as described above for FIGS. 13A-13E. When no further cutting is desired, the tool can be retrieved from the subject. As part of the ViV procedure, the new prosthetic heart valve in a crimped state can subsequently be advanced to the existing valvular structure with modified leaflets 14. The new valve is disposed within the valvular structure and expanded, such that the leaflets 14 are disposed on an external surface of the new valve frame. However, the cut leaflets 14 can allow blood to flow from the outflow end of the new prosthetic valve to the coronary arteries 22, 24 once the ViV procedure is completed. Alternatively or additionally, when the existing valvular structure is a BAV, the cut leaflets can allow for improved mounting of the cylindrical prosthetic valve, which would otherwise be compromised by the non-circular geometry of the native BAV.

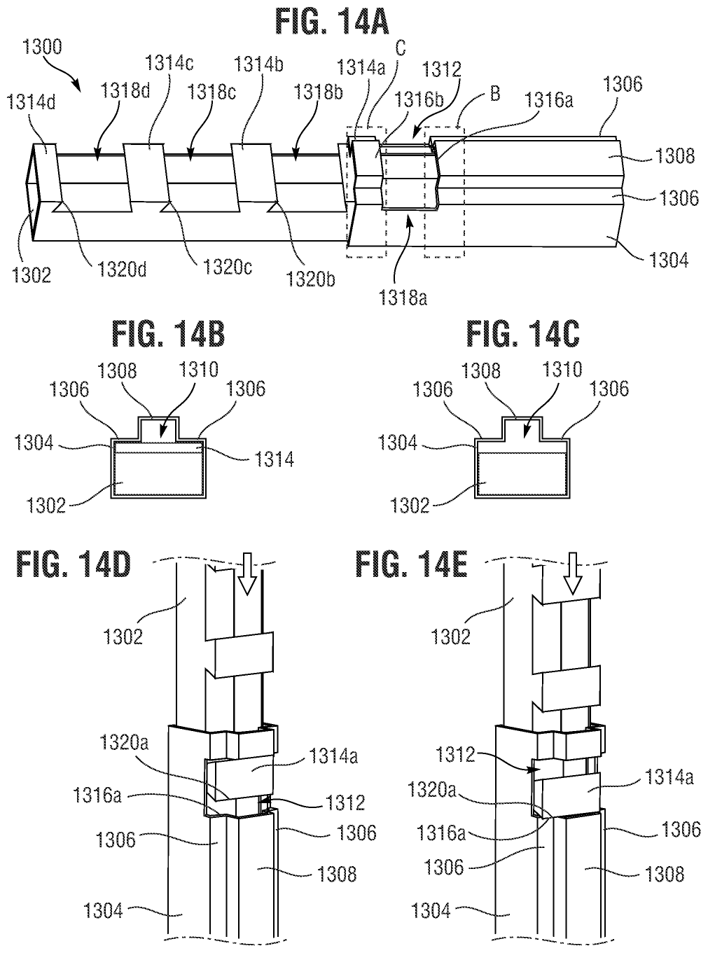

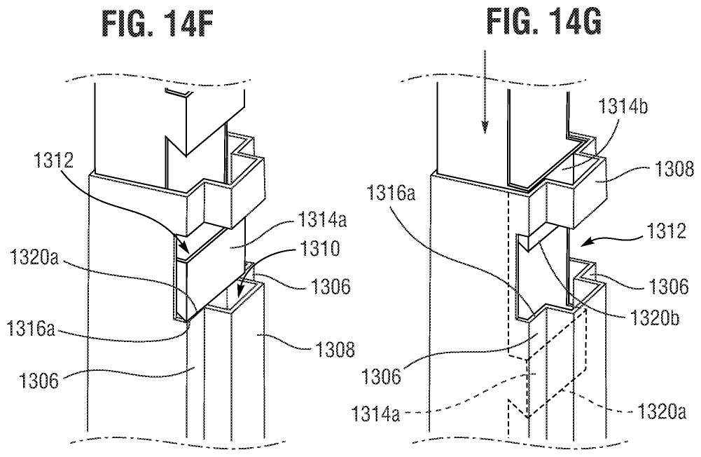

FIGS. 14A-14G illustrate a thirteenth exemplary tool 1300, which is configured to provide an extended cut in a leaflet of an existing valvular structure. The tool 1300 comprises an outer hollow shaft 1304 and an inner hollow shaft 1302. The inner shaft 1302 can be disposed within and movable relative to the outer shaft 1304 along an axial direction thereof. The outer shaft 1304 can have a shape in cross-section that is polygonal, for example, having eight sides. For example, the cross-section of the outer shaft 1304 can be composed of a substantially-rectangular main channel and a substantially-rectangular supplemental channel 1310 disposed along a cutting side of the outer shaft 1304. Thus, the combination of main channel and supplemental channel 1310 provides the cutting side of the outer shaft 1304 with a raised central portion 1308 between a pair of lateral flat portions 1306, (e.g., a fin profile) as illustrated in FIGS. 14A-14C. The cutting side of the outer shaft 1304 can have a first window 1312. The first window 1312 can have a distal edge 1316b (which follows a shape of the lateral flat portions 1306 and central portion 1308), a proximal edge 1316a (which follows a shape of the lateral flat portions 1306 and central portion 1308) separated from the distal edge 1316b along the axial direction, and a pair of axial edges extending the distal edge 1316b and the proximal edge 1316a.

The inner shaft 1302 can have a shape in cross-section that is substantially rectangular, as shown in FIGS. 14B-14C. Along a cutting side of the inner shaft 1302, a plurality of teeth 1314a-1314d can be disposed, with each tooth 1314 being separated from an adjacent tooth along the axial direction by a respective recess or inner window 1318a-1318d. Each tooth 1314a-1314d can have a respective cutting edge 1320a-1320d that faces toward the proximal direction. The cutting edge 1320a-1320d can comprise a sharpened edge or a blade. Alternatively or additionally, the proximal edge 1316a of the first window 1312 can comprise a sharpened edge or a blade. Although four teeth 1314a-1314d and four windows 1318a-1318d are illustrated in FIG. 14A, additional or fewer teeth and corresponding windows are also possible according to one or more contemplated embodiments.

In some embodiments, each tooth 1314 can be at the same height with respect to a side of the inner shaft 1302 opposite the cutting side. Accordingly, each tooth 1314 may be disposed such that its uppermost surface passes just underneath or in contact with an underside of the lateral flat portions 1306 when the inner shaft 1302 is disposed within the main channel of the outer shaft 1304. In other embodiments, the teeth 1314 may have different heights with respect to a side of the inner shaft 1302 opposite the cutting side. For example, heights of the teeth 1314 can get progressively less the closer they are to the distal end of the inner shaft 1302. Thus, distal tooth 1314d may have the smallest height and be disposed such that its uppermost surface is spaced a first distance from the underside of the lateral flat portions 1306 when the inner shaft 1302 is disposed within the main channel of the outer shaft 1304, and proximal tooth 1314a may have the greatest height and be disposed such that its uppermost surface passes just underneath or in contact with an underside of the lateral flat portions 1306 when the inner shaft 1302 is disposed within the main channel of the outer shaft 1304.

The inner shaft 1302 can be disposed within the outer shaft 1304 such that inner window 1318a adjacent to the proximal tooth 1314a is aligned with the first window 1312 of the outer shaft 1304, for example, as shown in FIG. 14A. The tool 1300 can be advanced in an ascending aorta of a subject toward a free end or edge of a leaflet (not shown) of the existing valvular structure. In particular, the cutting side of the tool 1300 can be oriented toward the leaflet such that a portion of the leaflet extends through the aligned windows 1312, 1318a. The inner shaft 1302 can then be retracted in the proximal direction relative to the outer shaft 1304, thereby exposing the proximal tooth 1314a as shown in FIG. 14D. Retraction of the inner shaft 1302 with respect to the outer shaft 1304 can be accomplished by holding the outer shaft 1304 in a fixed position and pulling the inner shaft 1304 in the proximal direction, holding the inner shaft 1302 in a fixed position and pushing the outer shaft 1304 in the distal direction, or a combination thereof.

With further retraction of the inner shaft 1302 in the proximal direction relative to the outer shaft 1304, the cutting edge 1320a of tooth 1314a of inner shaft 1302 approaches the proximal edge 1316a of the outer shaft 1304, as shown in FIGS. 14E-14F. A portion leaflet between the cutting edge 1320a and the proximal edge 1316a will initially be caught therebetween and then cut (by cutting edge 1320a and/or proximal edge 1316a) as the inner shaft 1302 is moved proximally. In particular, the portions of the proximal edge 1316a corresponding to lateral flat portions 1306 are sufficiently close to the passing cutting edge 1320a of tooth 1314a to provide cutting of the leaflet portions in contact therewith. However, the portions of the proximal edge 1316a corresponding to the raised central portion 1308 are spaced from the passing cutting edge 1320a of tooth 1314a and thus provide no or only minimal cutting of the leaflet. Thus, a central portion of the leaflet remains uncut and becomes disposed between the tooth 1314a and the internal walls of supplemental channel 1310. Further retraction of the inner shaft 1302 in the proximal direction, as shown in FIG. 14G, pulls the uncut portion of the leaflet along channel 1310 so that a different part of it can be cut by the next tooth 1314b. Each subsequent tooth 1314 can thus pull more of the leaflet into window 1312 for cutting.

Alternatively, as noted above, each subsequent tooth can be spaced at a different height (e.g., closer to lateral flat portions 1306 than previous teeth). In such an alternative configuration, the uncut portion of the leaflet can slide along in channel 1310 as the next tooth 1314b is advanced in the proximal direction. Since the second tooth 1314b is at a different height than the first tooth 1314a, a different part of the leaflet is presented to the cutting edge 1320b of the second tooth 1314b than was presented to the cutting edge 1320a of the first tooth 1314a. The cutting edge 1320b of the second tooth 1314b thus interacts with and cuts an uncut portion of the leaflet in a manner similar to that described above. Further retraction of the inner shaft 1302 in the proximal direction can cut the leaflet at different locations based on the different heights of subsequent teeth 1314c-1314d.

If additional cutting of the same or different leaflets of the existing valvular structure is desired, for example, to cut additional portions of the same leaflet or to cut portions of other leaflets for both coronary arteries 22, 24, then the thirteenth tool or variations thereof can be repositioned (e.g., laterally moving the outer shaft). For example, the inner shaft 1302 can be moved distally to its initial position (e.g., with proximal tooth 1314a disposed distally of window 1312 of the outer shaft 1304), thereby releasing the uncut leaflet portion previously retained in channel 1310. The cutting of the next leaflet can then be performed in a similar manner as described above for FIGS. 14A-14G.

When no further cutting is desired, the tool can be retrieved from the subject. As part of the ViV procedure, the new prosthetic heart valve in a crimped state can subsequently be advanced to the existing valvular structure with modified leaflets 14. The new valve is disposed within the valvular structure and expanded, such that the leaflets 14 are disposed on an external surface of the new valve frame. However, the cut leaflets 14 can allow blood to flow from the outflow end of the new prosthetic valve to the coronary arteries 22, 24 once the ViV procedure is completed. Alternatively or additionally, when the existing valvular structure is a BAV, the cut leaflets can allow for improved mounting of the cylindrical prosthetic valve, which would otherwise be compromised by the non-circular geometry of the native BAV.

In some embodiments, one or more cutting elements can be positioned with respect to a center of an existing valvular structure. A self-expanding stent or frame can contact the surrounding structure (e.g., the walls of the blood vessel) proximal to the existing valvular structure to stabilize a position of the cutting elements with respect to the valvular structure. For example, each cutting element can be supported on or be a part of an axially-extending crossing catheter. Each cutting element can extend radially from a main body of the crossing catheter, such that when the crossing catheter is moved axially through the existing valvular structure, the cutting element comes into contact with and slice through one or more leaflets of the existing valvular structure. In some embodiments, the crossing catheter has a cutting element corresponding to each leaflet, such that multiple leaflets can be cut simultaneously by movement of the crossing catheter.

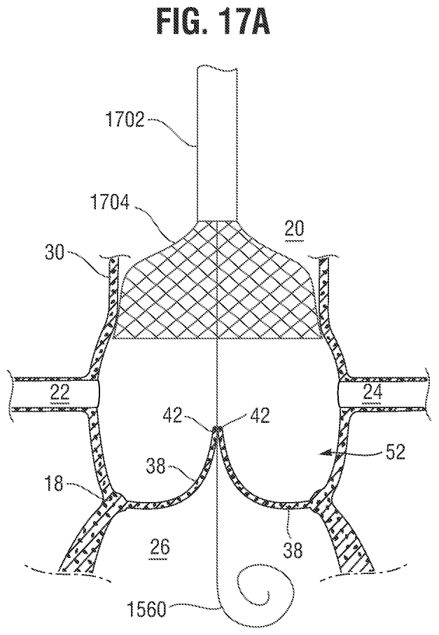

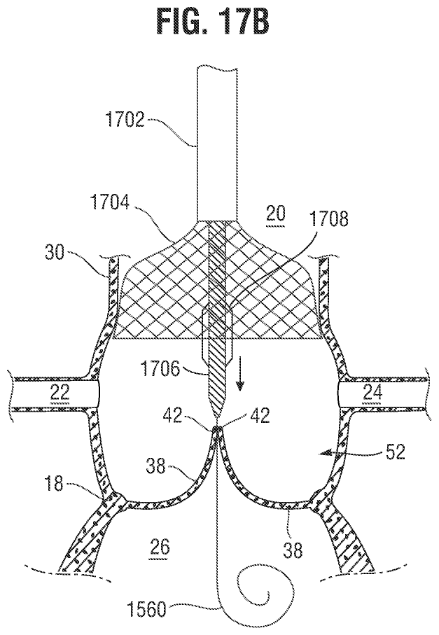

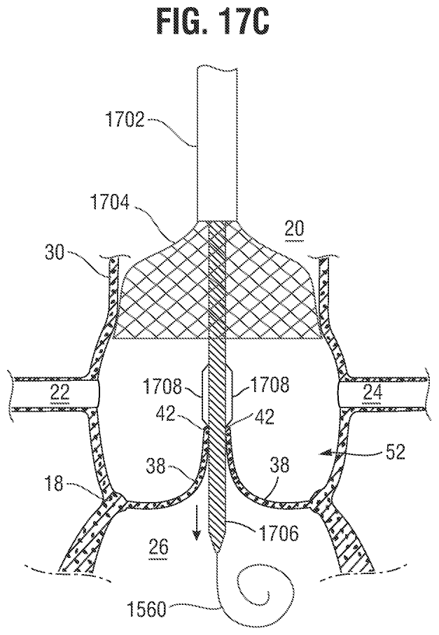

For example, FIGS. 17A-17C illustrate a fourteenth exemplary tool, which employs a crossing catheter to cut one or more leaflets 38 of the existing valvular structure. The tool comprises a delivery system 1702 (e.g., a catheter, capsule, sheath, or other delivery shaft) with a distal end thereof configured to be disposed within an ascending aorta of a subject using a guide wire 1560. The tool can further comprise a stabilizing member, such as in the form of a frame or stent 1704, which may be formed of a shape-memory alloy. The stabilizing stent 1704 may be initially disposed within the delivery system 1702 in a compressed state. When the stent 1704 is released from the delivery system 1702, the stent 1704 can automatically expand from the compressed state to an unbiased or free state, where a portion of the stent 1704 (e.g., a circumferential surface of a distal end portion) contacts an adjacent wall 30 of the aorta, as shown in FIG. 17A. In other embodiments, the stent 1704 can be a mechanically expandable stent that can be expanded using mechanical actuators. The stent 1704 can contact the aortic wall 30 just above the existing valvular structure 52, for example, at, near, or within the sinotubular junction. At least a proximal portion of the stent 1704 can be retained in the delivery system 1702 or otherwise coupled thereto, so as to provide a stabilizing force to center the delivery system 1702 with respect to valvular structure 52.

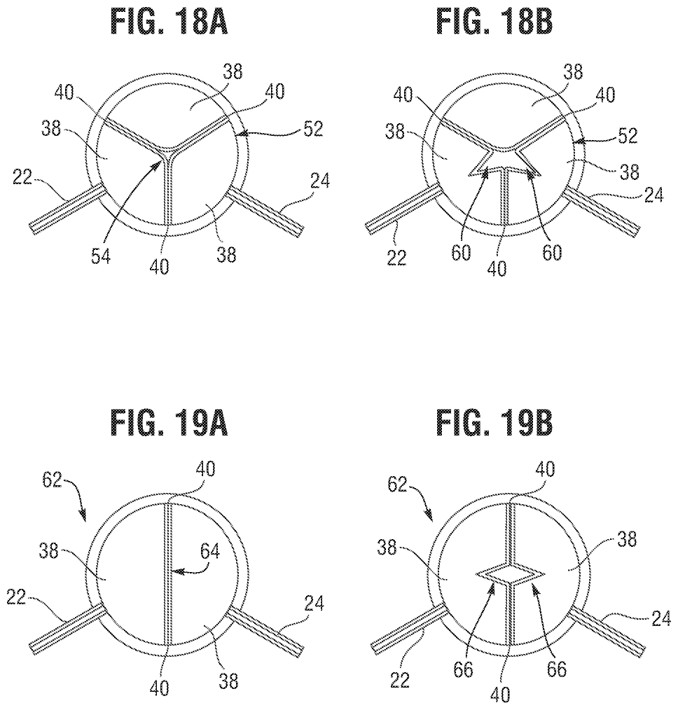

With the stabilizing stent 1704 in contact with the aortic wall 30, a crossing catheter 1706 can be distally advanced from the distal end of the delivery system 1702 toward a center of the valvular structure (e.g., center 54 of a normal aortic valve in FIG. 18A or center 64 of a bicuspid aortic valve in FIG. 19A). Alternatively, in some embodiments, the crossing catheter 1706 be positioned offset from a center of the valvular structure (e.g., center 54 of a normal aortic valve in FIG. 18A or center 64 of a bicuspid aortic valve in FIG. 19A), for example, such that the gashes produced are more closely positioned to the location of the coronary arteries.

In the example illustrated in FIG. 17B, the crossing catheter is aligned to pass between free ends 42 of adjacent leaflets 38. The crossing catheter 1706 can include one or more cutting elements 1708 that slice through portions of leaflet 38 that come into contact therewith during axial movement (e.g., distal movement) of the catheter 1706 through the existing valvular structure 52, as shown in FIG. 17C. For example, each cutting element 1708 can include a sharp edge or blade that projects radially from a main body of the catheter 1706. Alternatively or additionally, each cutting element 1708 can be configured to cut leaflets in contact therewith using electrical energy applied thereto. Although a particular geometry for the cutting element 1708 has been illustrated in FIG. 17C, other geometries are also possible according to one or more contemplated embodiments. For example, the cutting element 1708 can be tapered along the axial direction, such that the cutting element 1708 projects farther from the main body of the catheter 1706 at its proximal end than its distal end. Accordingly, a length of the gash formed in the leaflet 38 by contact with the cutting element 1708 can be increased as the crossing catheter 1706 moves distally further through the valvular structure.

In some embodiments, the catheter 1706 can include a cutting element 1708 for each leaflet 38 of the valvular structure 52, so as to simultaneously cut multiple leaflets at the same time. For example, when existing valvular structure 52 is a native aortic valve with three leaflets 38, the catheter 1706 can have three cutting elements 1708, each disposed at a 120° interval with respect to the other cutting elements. Alternatively, in some embodiments, the catheter 1706 may have a pair of cutting elements 1708 even though the native aortic valve has three leaflets 38. For example, the pair of cutting elements 1708 can be positioned to simultaneously cut leaflets 38 that would face the ostia of coronary arteries 22, 24 after implantation of the new prosthetic valve. Alternatively, when the existing valvular structure is a native aortic valve with two leaflets 38 (e.g., a bicuspid aortic valve (BAV)), the catheter 1706 can have two cutting elements 1708. For example, the pair of cutting elements 1708 can be positioned at a 180° interval with respect to the other so as to simultaneously cut leaflets 38 on opposite sides of the catheter 1706. Additional or fewer cutting elements 1708 are also possible according to one or more contemplated embodiments. For example, the number of cutting elements 1708 can be less than the number of leaflets 38, and the catheter 1706 can be repositioned (e.g., rotated with respect to the valvular structure) and moved axially (e.g., either proximally or distally) through the valvular structure 52 to effect cutting of additional leaflets.

When no further cutting is desired, the crossing catheter 1706 and the stabilizing stent 1704 can be fully retracted into the delivery system 1702. The tool can then be retrieved from the subject. The new prosthetic heart valve in a crimped state can subsequently be advanced to the existing valvular structure with modified leaflets 38. The new valve is disposed within the valvular structure 52 and expanded, such that the leaflets 38 are disposed on an external surface of the new valve frame. However, the one or more gashes formed in the leaflets 38 allow blood to flow from the outflow end of the new prosthetic valve to the coronary arteries 22, 24 once the new valve installation procedure is completed. Alternatively or additionally, when the existing valvular structure is a BAV, the one or more gashes formed in the leaflets can allow for improved mounting of the cylindrical prosthetic valve, which would otherwise be compromised by the non-circular geometry of the native BAV.

In some instances, exemplary tools can be configured to cut out a portion of a leaflet of an existing valvular structure and to remove the cut-out portion from the subject. For example, a tool can have a coring tip with a sharp edge surrounding an open end. The coring tip can be coupled to a torque shaft and can extend from a catheter (also referred to as a sheath or shaft). In some embodiments, the coring tip can be integrated with existing components of a delivery system for implantation of the prosthetic heart valve. For example, the coring tip can be attached to a distal end of the balloon shaft (also referred to as balloon catheter) or another shaft of a delivery system, such as the Edwards Commander delivery system, which is disclosed in U.S. Patent Application Publication No. 2013/0030519, which is incorporated herein by reference. A vacuum can be applied to the coring tip via the torque shaft, such that a surface of one or more leaflets of an existing valvular structure contacts the sharp edge. Rotation of the torque shaft causes the sharp edge of the coring tip to slice through the leaflet to create a hole therein. The cutout portion of the leaflet is drawn under vacuum into the interior of the coring tip, for example, to minimize debris that may adversely affect the subject or subsequent prosthetic heart valve implantation. Moreover, because any portion of the leaflet may be engaged by the coring tip, high accuracy positioning of the tool with respect to the leaflet can be avoided.

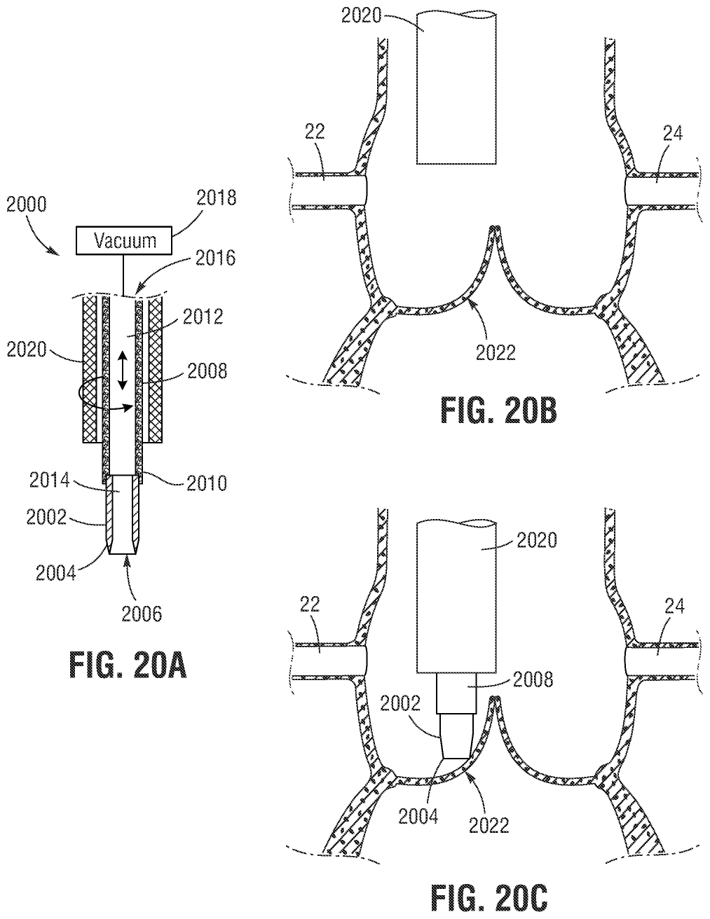

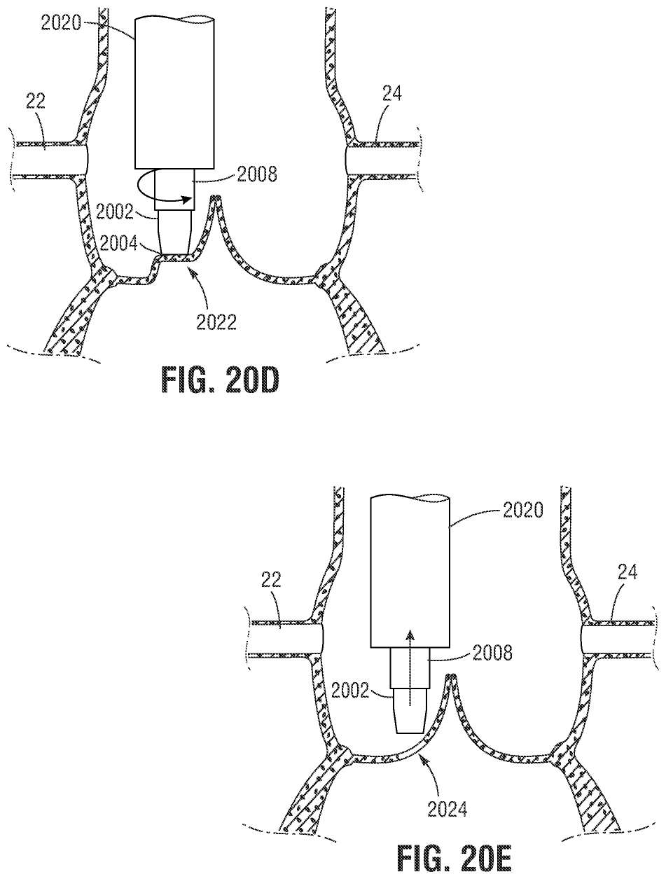

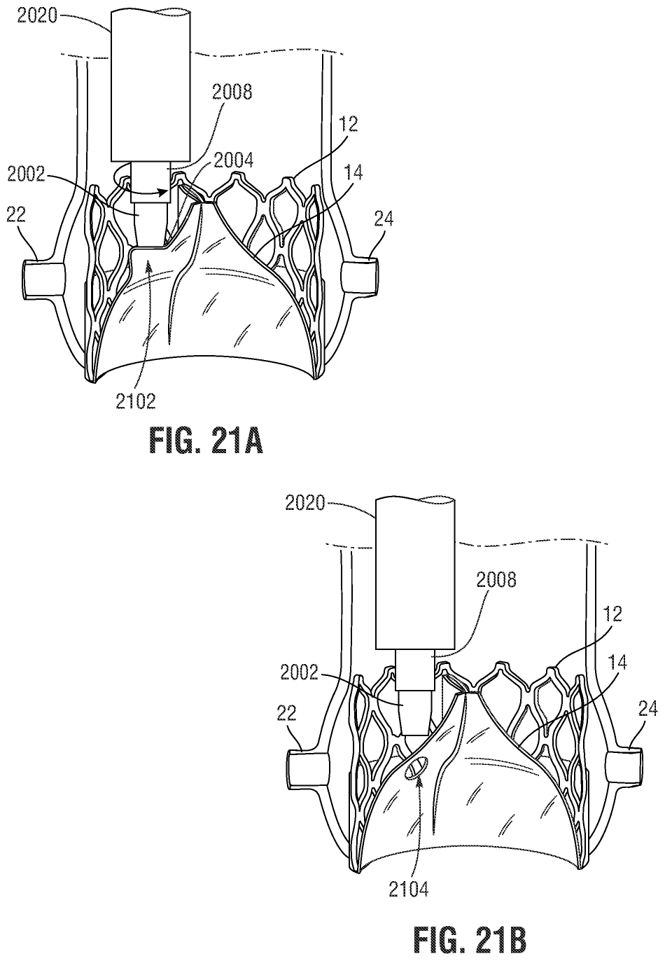

For example, FIGS. 20A-21B illustrate a fifteenth exemplary tool 2000, which employs a vacuum-assisted coring tip to create a hole within a leaflet of an existing valvular structure. The tool 2000 comprises a coring tip 2002 coupled to a distal end 2010 of a torque shaft 2008. The coring tip 2002 can have a cutting edge 2004 (e.g., a sharpened, beveled edge, a serrated edge, or any other type of cutting edge) surrounding a substantially-circular opening 2006 at a distal end thereof. For example, in some embodiments, the coring tip 2002 can comprise a hypotube formed of a metal or metal alloy (e.g., surgical steel, titanium, and nickel-titanium) and having a sharpened circumferential edge an axial end thereof. The torque shaft 2008 can be coupled to a vacuum source 2018 (e.g., vacuum pump) at its proximal end 2016, such that a vacuum (e.g., a pressure less than that of the environment surrounding the coring tip 2002) is generated at opening 2006 via internal volume 2014 of the coring tip 2002 and connecting internal volume 2012 of the torque shaft 2008.

The coring tip 2002 can be rigidly connected to the torque shaft 2008 such that the coring tip 2002 and the torque shaft 2008 move together. For example, the torque shaft 2008 can be movable within the sheath 2020 along a longitudinal axis thereof so as to move the coring tip 2002 between a first location disposed within an internal volume of the sheath 2020 and a second location exposed from the distal end of the sheath 2020. In some embodiments, the torque shaft 2008 is rotatable about its longitudinal axis (independent of the sheath 2020 or in conjunction with rotation of the sheath 2020) in order to spin the sharp edge 2004 in a circumferential direction of the coring tip 2002, for example, to assist in cutting through tissue (e.g., leaflet) in contact therewith. Rotation of the torque shaft 2008 and/or sheath 2020 can be effected by operator manipulation of an appropriate control mechanism at a corresponding proximal end thereof, for example, by turning of a knob.

For example, an operator can position a distal end of the sheath 2020 with respect to a resection target 2022 of a leaflet of a native aortic valve, as shown in FIG. 20B. The coring tip 2002 can then be extended distally toward the resection target 2022 from the sheath 2020 such that at least the cutting edge 2004 is exposed, as shown in FIG. 20C. The vacuum source can then be engaged, and the resection target 2022 brought into contact with the cutting edge 2004, as shown in FIG. 20D. With the vacuum engaged, the torque shaft 2008 can be rotated about its axis, thereby causing the cutting edge 2004 to slice through the leaflet tissue in contact therewith and forming an opening 2024 (e.g., a substantially circular hole) in the leaflet, as shown in FIG. 20E. The vacuum sucks the cut portion of the leaflet, and any other debris generated by the cutting process, into the internal volume 2014 of the coring tip 2002 (and optionally further into internal volume 2012 of the torque shaft 2008), thereby reducing the risk of the leaflet cutting procedure (e.g., due to clotting or other complications that may result from the debris). Alternatively, the coring tip 2002 may remain within sheath 2020 during the cutting procedure, for example, by relying on vacuum application to draw a portion of the leaflet into the distal end of the sheath 2020 and into contact with the cutting edge 2004 within the sheath 2020.

Although the above description has focused on the use of the fifteenth tool 2000 to cut leaflets of a native aortic valve, embodiments of the disclosed subject matter are not limited thereto. Indeed, in some embodiments, the fifteenth tool 2000 can be used to modify one or more leaflets of a valvular structure of a previously-implanted prosthetic heart valve in order to prevent, or at least reduce the risk of, obstruction of blood flow to the coronary arteries 22, 24 after a new prosthetic heart valve is installed within the existing valve. For example, FIGS. 21A-21B illustrate engagement of the coring tip 2002 with a resection target 2102 of a leaflet 14 of an implanted prosthetic heart valve and cutting thereof to form an opening 2104, in a manner similar to that described above for FIGS. 20A-20E.

If additional cutting of the same or different leaflets is desired, for example, to provide additional openings in the same leaflet or to provide openings for both coronary arteries 22, 24, the fifteenth tool can be repositioned (e.g., by laterally moving the sheath 2020) with or without prior retraction of the coring tip 2002 into sheath 2020. The cutting of the next resection target can then be performed in a similar manner as described above for FIGS. 20A-20E. In some embodiments, the vacuum can be disengaged or paused after the coring tip 2002 has cut through the resection target to allow for repositioning with respect to the next resection target or leaflet. Alternatively or additionally, the vacuum can remain engaged during repositioning. For example, when the vacuum is initially engaged, the resection target can displace into contact with the cutting edge 2004. However, once the cutting is complete, the newly formed opening (e.g., opening 2024 or opening 2104) may allow the leaflet to revert to its original position despite the continued application of vacuum to the coring tip 2002. The coring tip 2002 can then move to the next leaflet for additional cutting, where the continued vacuum application again pulls the new resection target into contact with the cutting edge 2004.

When no further cutting is desired, the coring tip 2002 can be retracted into the sheath 2020, and the tool 2000 retrieved from the subject. As part of the ViV procedure, the new prosthetic heart valve in a crimped state can subsequently be advanced to the existing valvular structure with modified leaflets (e.g., either the native valve of FIGS. 20B-20E or the prosthetic valve of FIGS. 21A-21B). The new valve is disposed within the valvular structure and expanded, such that the leaflets are disposed on an external surface of the new valve frame. However, the one or more openings holes formed in the leaflets allow blood to flow from the outflow end of the new prosthetic valve to the coronary arteries 22, 24 once the ViV procedure is completed.

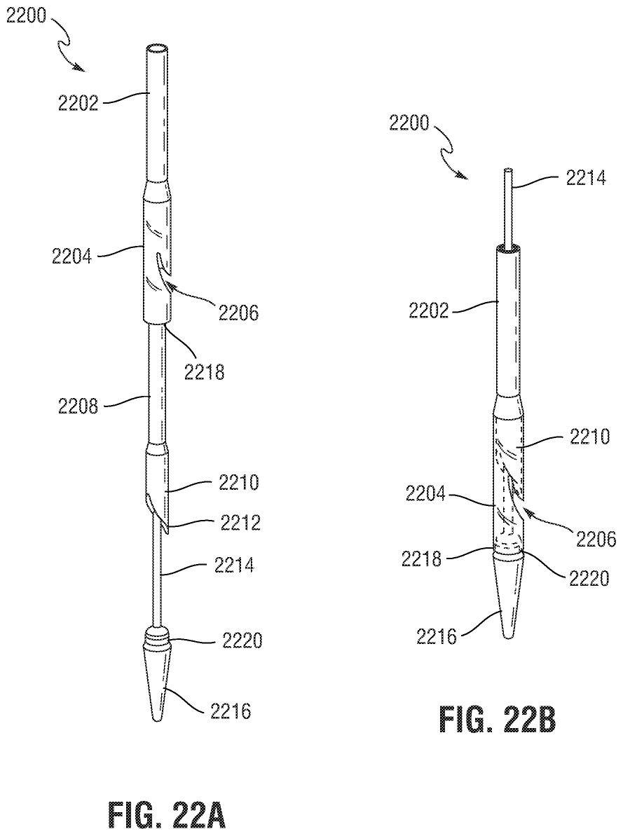

In some instances, exemplary tools can be configured to cut out a portion of a leaflet of an existing valvular structure and to retain the cut-out portion for removal from the subject with the tool. By retaining the cut-out portion, the amount of debris that may adversely affect the subject or subsequent prosthetic heart valve implantation can be minimized or at least reduced. For example, FIGS. 22A-22B illustrate a sixteenth exemplary tool 2200 that can provide such functions. The tool 2200 comprises a hollow, outer shaft 2202 and a hollow, inner shaft 2208. The outer shaft 2202 can include at its distal end a capsule portion 2204. In some embodiments, the capsule portion 2204 can have a diameter larger than that of the remainder of the outer shaft 2202, and the capsule portion 2204 can be connected to the remainder of the outer shaft 2202 by a flared portion, as shown in FIG. 22A. The inner shaft 2208 can include at its distal end a blade member 2210. In some embodiments, the blade member 2210 can have a diameter larger than that of the remainder of the inner shaft 2208, and the blade member 2210 can be connected to the remainder of the inner shaft 2208 by a flared portion, as shown in FIG. 22A.

The capsule portion 2204 can have a curved slot 2206 in a circumferential wall thereof, thereby providing access to an internal volume of the capsule portion 2204. For example, the curved slot 2206 can have a crescent-shaped profile, with opposite ends of the slot 2206 being arranged proximally and a center of the slot 2206 being arranged distally. In some embodiments, a width of the slot 2206 (e.g., as measured between facing edges of the slot) can be substantially the same along its length from one end to the other end. Alternatively, in some embodiments, the width of the slot 2206 can vary along its length, for example, with a center of the slot 2206 having a largest width and ends of the slot 2206 having the smallest width. In some embodiments, the geometry of the slot 2206 (e.g., the profile curvature, width, length, etc.) is selected to match the geometry of a leaflet to be accommodated therein. As described in more detail below, a portion of the leaflet fits into and extends along the slot 2206 when the capsule 2204 slides over the free end of the leaflet in order to retain the leaflet for cutting thereof by a blade member 2210 within the capsule portion 2204.

A distal edge 2212 of the blade member 2210 can be configured as a cutting edge, for example by being sharpened and/or having a serrated edge. A profile of the distal edge 2212 can follow a profile of the curved slot. For example, in some embodiments, when the blade member 2210 is displaced axially past slot 2206, the distal edge 2212 initiates contact with each portion of the leaflet retained within slot 2206 substantially simultaneously. Alternatively, in some embodiments, the distal edge 2212 can have a profile different from that of the curved slot 2206. For example, in some embodiments, the distal edge 2212 can be substantially perpendicular to the axial direction of the outer shaft 2202. In such a configuration, when the blade member is displaced axially past slot 2206, the distal edge initiates contact with portions of the leaflet retained at opposite ends of slot 2206 before a portion of the leaflet retained at a middle of the slot 2206.

The tool 2200 further comprises a nosecone 2216 at a distal end of a guidewire lumen 2214. The inner shaft 2208 and the outer shaft 2202 are disposed over the guidewire lumen 2214 in a coaxial arrangement, with the inner shaft 2208 being disposed within and movable with respect to the outer shaft 2202. For example, in some embodiments, the inner shaft 2208 can be moved axially within the outer shaft 2202, in particular, to move the blade member from a first location proximal of the curved slot 2206 to a second location distal of the curved slot 2206. Alternatively or additionally, in some embodiments, the inner shaft 2208 can be rotated about at a longitudinal axis thereof within the outer shaft 2202, for example, to facilitate cutting through a portion of the leaflet that extends through slot 2206.

In the fully-assembled initial configuration of FIG. 22B, the distal end 2218 of the capsule portion 2204 abuts a proximal end 2220 of the nosecone 2216, thereby defining an enclosed volume accessible via curved slot 2206. In addition, in this fully-assembled initial configuration, the blade member 2210 may be disposed proximal of the curved slot 2206, thereby concealing the cutting edge 2212 from incidental, unintended contact with surrounding tissue. In some embodiments, in the fully-assembled initial configuration, the flared portion of the inner shaft 2208 can be disposed within the flared portion of the outer shaft 2202 (e.g., at a common axial location), as shown in FIG. 22B.

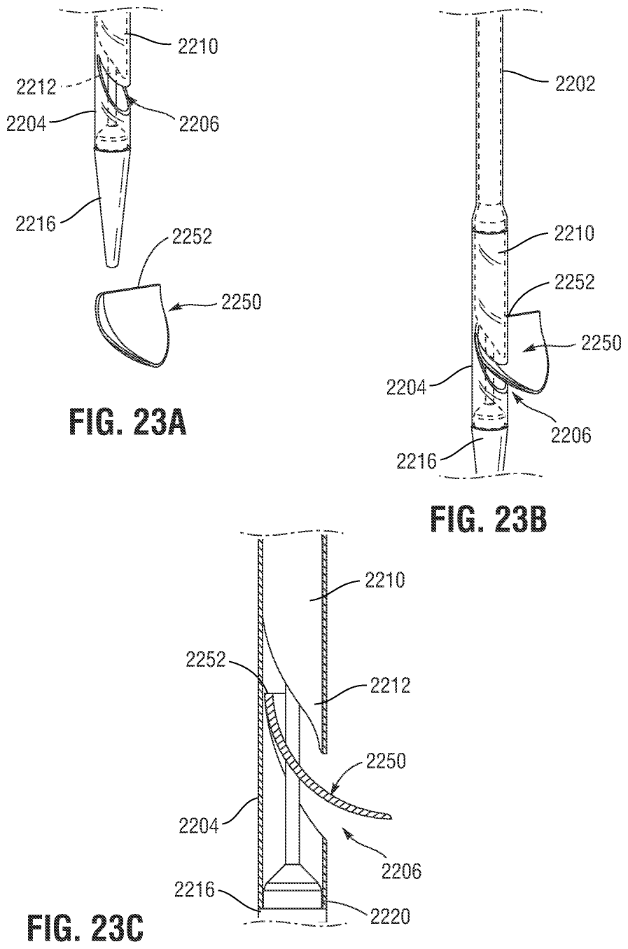

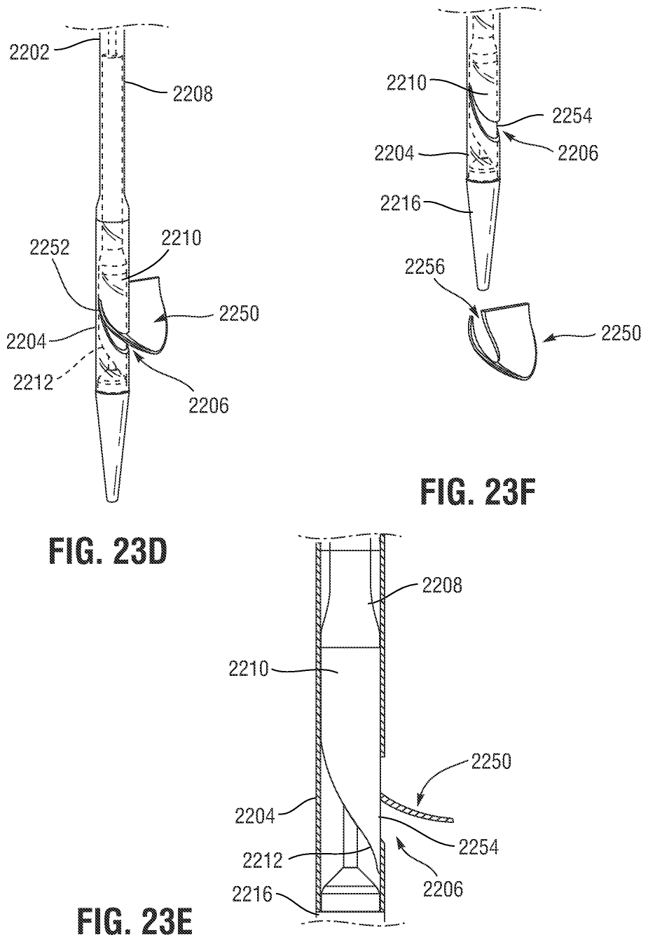

FIGS. 23A-23F illustrate an exemplary operation of the sixteenth tool 2200 in cutting a leaflet of an existing valvular structure (e.g., a native aortic valve or a previously-implanted prosthetic heart valve). Prior to FIG. 23A, the nosecone 2216 and guidewire lumen 2214 can be advanced via the ascending aorta of a subject to a position with respect to one leaflet 2250 (other leaflets not illustrated for clarity). The inner shaft 2208 and the outer shaft 2202 can then be advanced together over guidewire lumen 2214 toward the leaflet 2250, with the blade member 2210 being positioned proximal to the curved slot 2206 during this delivery. As such, the nosecone 2216 can be positioned within the subject's anatomy first (e.g., via a guidewire extending through the nosecone 2216 and lumen 2214) in order to serve as a guide for subsequent advancement of the inner and outer shafts of the tool 2200 over the attached guidewire lumen 2214. Once the distal end 2218 of the capsule portion 2204 of the outer shaft 2202 abuts the nosecone 2216, the sixteenth tool 2200 attains the fully-assembled initial configuration, as shown in FIG. 23A.

In this state, the tool can be advanced toward a leaflet 2250, and the capsule portion 2204 can slide along the leaflet 2250 such that a free end or edge portion 2252 of the leaflet 2250 enters into the curved slot 2206, as shown in FIGS. 23B-23C. The sliding of capsule portion 2204 over the leaflet to engage with an edge thereof can allow for easier operation and avoid a requirement for high accuracy positioning. The outer shaft 2202, in particular the capsule portion 2204, can be maintain its position with respect to leaflet 2250 while the inner shaft 2208, in particular the blade member 2210 is moved distally (and optionally rotated about its axis) into contact with the portion of the leaflet 2250 retained in slot 2206. The cutting edge 2212 of the blade member 2210 can slice through the portion of the leaflet 2250 as the cutting edge 2212 moves distal of the curved slot 2206, thereby severing a portion of the leaflet 2250. Further movement of the blade member 2210 distally along the axial direction causes a sidewall 2254 of the blade member 2210 to block the slot 2206 as shown in FIGS. 23D-23E, thereby retaining the severed portion of the leaflet 2250 within the capsule. The tool 2200 can then be repositioned or retracted, for example, as shown in FIG. 23F.

If additional cutting of the same or different leaflets of the existing valvular structure is desired, for example, to cut additional portions of the same leaflet or to cut portions of other leaflets for both coronary arteries, then the sixteenth tool or variations thereof can be repositioned (e.g., laterally moving the outer shaft). The cutting of the next leaflet can then be performed in a similar manner as described above for FIGS. 23A-23F. However, in some embodiments, after the cutting of the leaflet 2250, the tool 2200 can be retracted from the subject, for example, to allow for disposal of the severed portion of the leaflet 2250. In such embodiments, the capsule portion 2204 can remain in contact with the nosecone throughout the cutting operation and retraction, and the slot 2206 can be closed off by the sidewall 2254 of the blade member 2210. Accordingly, the severed leaflet portion can be kept within the capsule portion 2204 and not otherwise exposed to the subject's bloodstream in order to avoid, or at least reduce, the risk of procedural complications (e.g., clotting). Moreover, in some embodiments, the cutting edge 2212 can be offset from the curved slot 2206 along the axial direction of the outer shaft 2202 during all phases of operation except the cutting phase. For example, during the advancing to position the leaflet 2250 within the curved slot 2206, the cutting edge 2212 is disposed proximal of the slot 2206, and after the cutting of the leaflet 2250, the cutting edge 2212 is disposed distal of the slot 2206. Accordingly, the cutting edge 2212 is concealed within the capsule portion 2204 before and after the cutting phase, thereby avoiding, or at least reducing, the risk of inadvertent cutting of tissue.

When no further cutting is desired, the tool can be retrieved from the subject. As part of the ViV procedure, the new prosthetic heart valve in a crimped state can subsequently be advanced to the existing valvular structure with modified leaflets. The new valve is disposed within the valvular structure and expanded, such that the leaflets are disposed on an external surface of the new valve frame. However, the gash or recessed portion 2256 formed in one or more of the leaflets 2250 can allow blood to flow from the outflow end of the new prosthetic valve to the coronary arteries once the ViV procedure is completed. Alternatively or additionally, when the existing valvular structure is a BAV, the cut leaflets can allow for improved mounting of the cylindrical prosthetic valve, which would otherwise be compromised by the non-circular geometry of the native BAV.

In any of the above noted examples, the modification of the existing valvular structure by any of the first through sixteenth tools can be performed as part of, before, or after a balloon annular valvuloplasty procedure on the existing valvular structure, for example, to prepare an existing heart valve (e.g., native aortic valve or previously implanted prosthetic valve) for subsequent implantation of a new prosthetic valve. Alternatively, in any of the above noted examples, the modification of the existing valvular structure by the tool can be performed as part of or preceding a transcatheter aortic valve implantation (TAVI) or transcatheter aortic valve replacement (TAVR) procedure, for example, to replace a native aortic valve or to replace a failing prosthetic valve (e.g., ViV procedure).

In some embodiments, any of the first through sixteenth tools can be used to modify the valvular structure of a previously-installed prosthetic heart valve, for example, as part of a valve-in-valve procedure. In other embodiments, any of the first through sixteenth tools can be used to modify the native valvular structure, for example, a normal aortic valve 52 having three leaflets 38 (as shown in FIG. 18A). As shown in FIG. 18B, the modification can include forming one or more gashes 60 in leaflets 38 that would face and potentially block the ostia of coronary arteries 22, 24 after installation of a new prosthetic heart valve within native valve 52. Other modifications to the leaflets 38 and/or commissures 40 besides the illustrated gashes 60 are also possible, for example, as described in detail above with respect to examples of the individual tools.

In still other embodiments, any of the first through sixteenth tools can be used to modify the native valvular structure of an abnormal aortic valve, for example, a BAV 62 having two leaflets 38 (as shown in FIG. 19A). Since the leaflets 38 of the BAV 62 can be relatively stiff and would normally define a non-circular mounting cross-section, implantation of a prosthetic heart valve within the native BAV 62 may be at risk for annular rupture and/or reduced hemodynamic performance. The modification can include forming one or more gashes 66 in leaflets 38, as shown in FIG. 19B, thereby allowing the BAV 62 to accommodate the cylindrical profile of the new prosthetic heart valve and avoiding the risks associated with implantation in a native BAV. Other modifications to the leaflets 38 and/or commissures 40 of BAV 62 besides the illustrated gashes 66 are also possible, for example, as described in detail above with respect to examples of the individual tools.

Since modification of the leaflets may compromise the ability of the existing valvular structure to function properly, it may be desirable to minimize, or at least reduce, a time period between the modification and subsequent implantation of the new prosthetic valve. Thus, in any of the above noted examples, the modification of the existing valvular structure by any of the first through sixteenth tools can be performed during implantation of, or between expansion stages in implanting, a new prosthetic valve within the existing heart valve (e.g., native aortic valve or previously-implanted prosthetic valve). For example, a mechanically-expandable prosthetic heart valve can be partially expanded within the existing valvular structure prior to modification of the existing valvular structure. The partial-expansion pushes the leaflets of the existing valvular structure radially outward into an annular region between a circumferential surface of the valve and the surrounding structures (e.g., the native anatomy or the frame of a previously implanted prosthetic valve). However, the partial-expansion allows the leaflets in the annular region to remain accessible from the ascending aorta. Accordingly, the leaflets can be modified by the tool in the ascending aorta, and then the prosthetic heart valve can be fully expanded to its implanted configuration soon after the modification, thereby minimizing or reducing a time of valve insufficiency due to the modification.

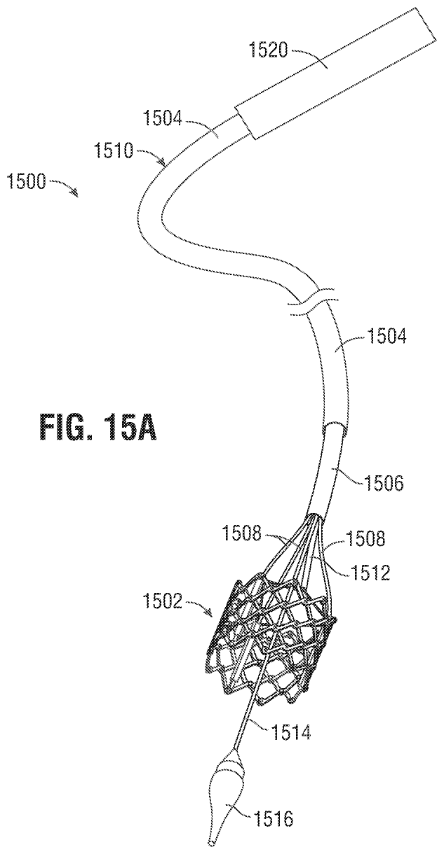

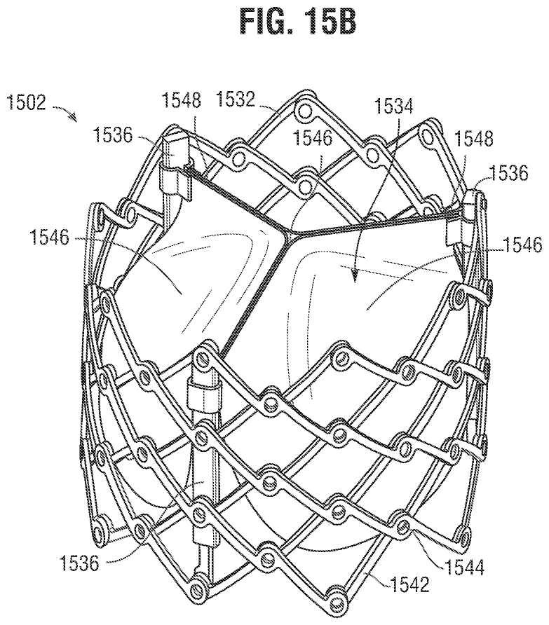

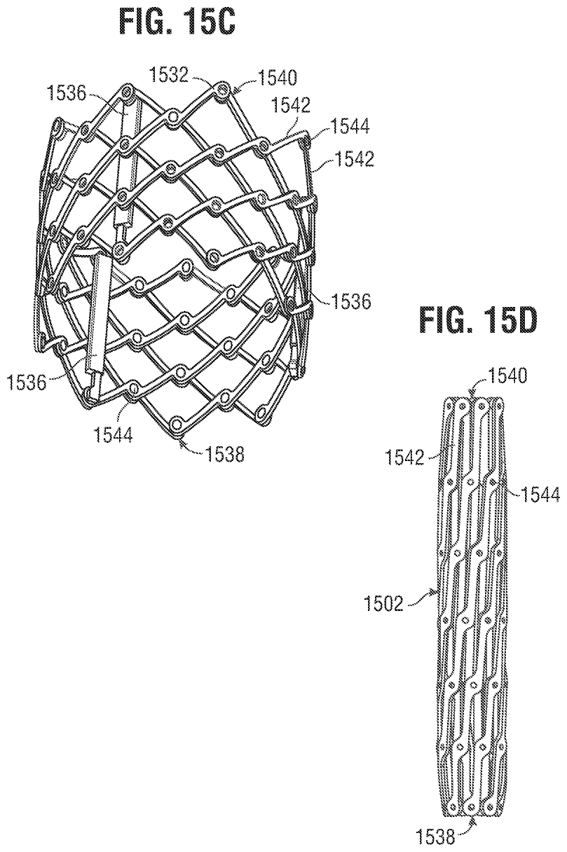

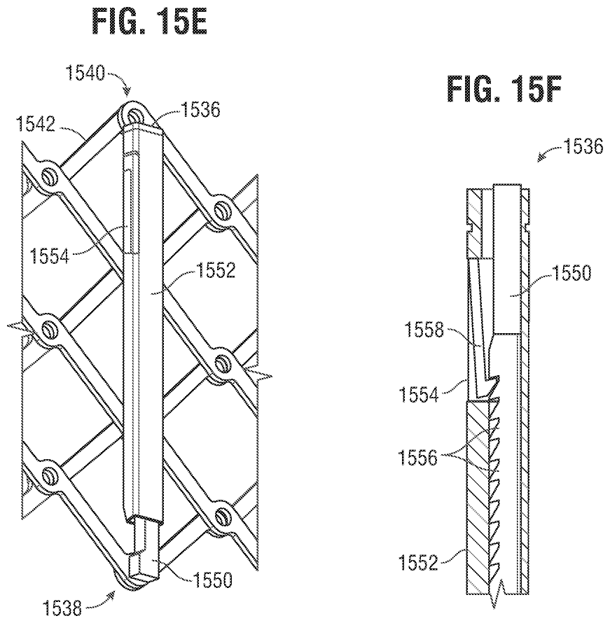

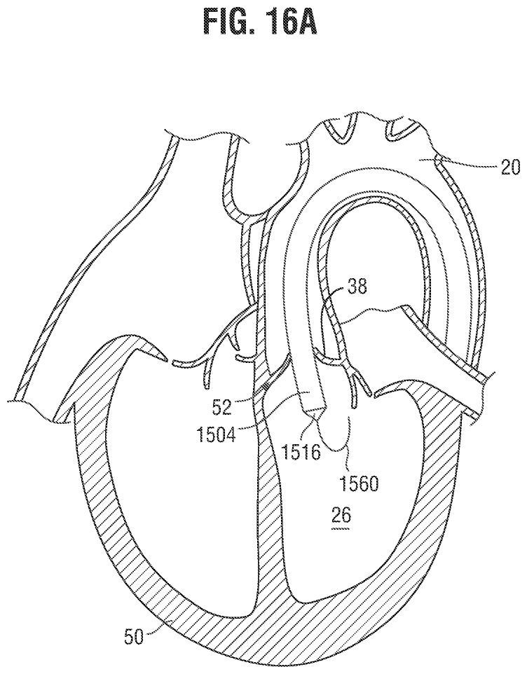

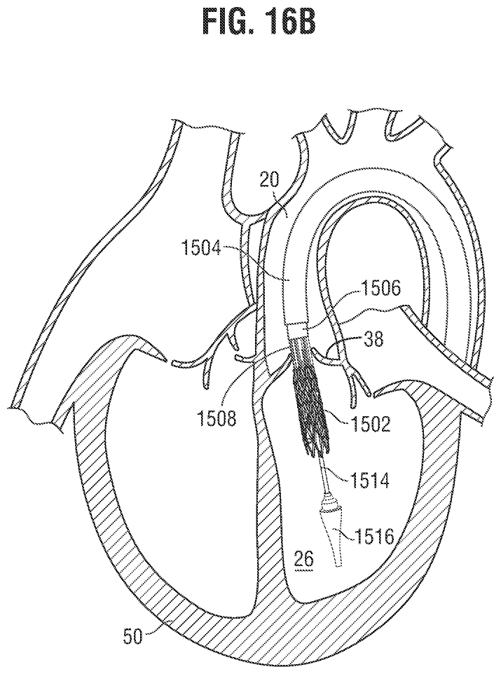

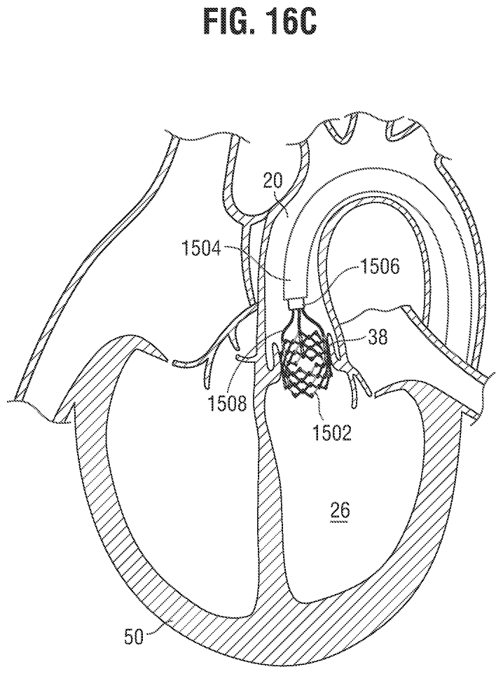

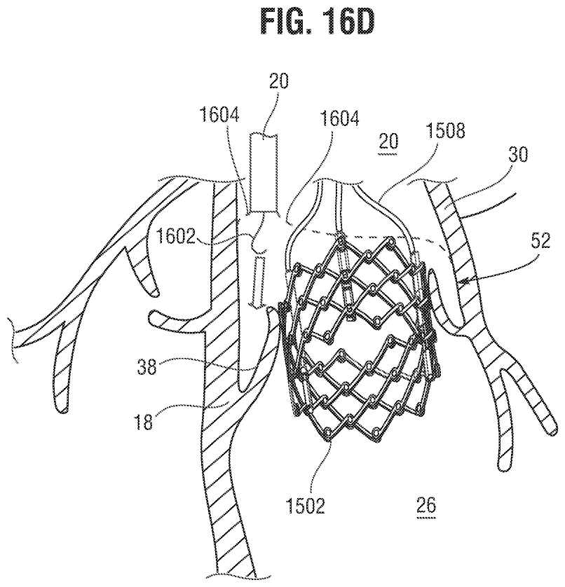

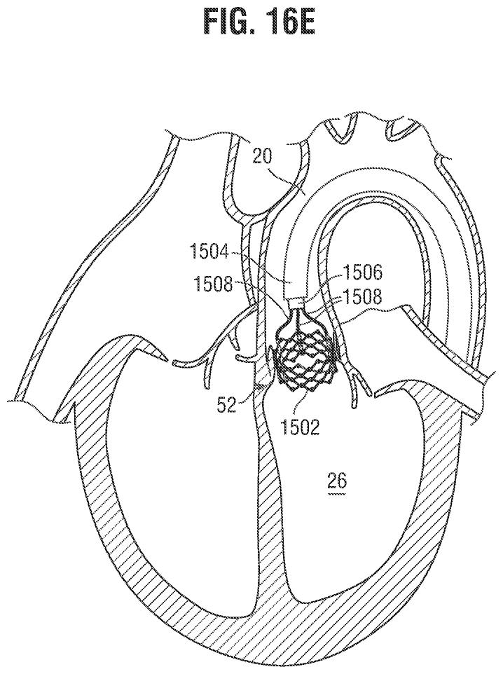

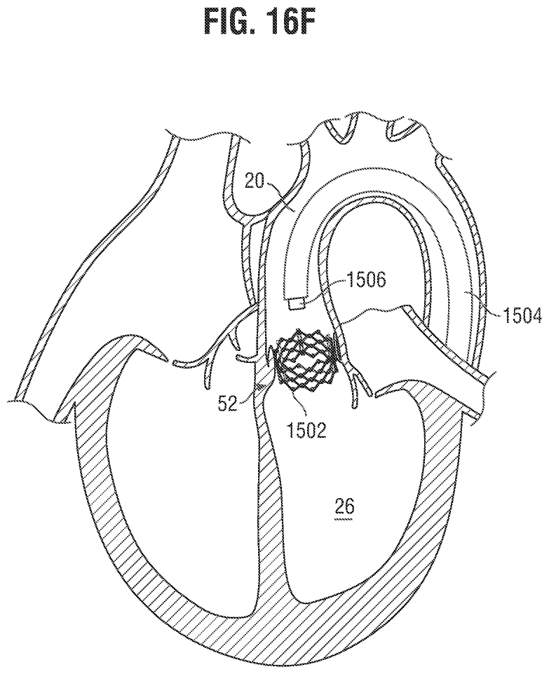

FIG. 15A shows an example of a delivery assembly 1500 that can be used to provide a partially-expanded prosthetic heart valve prior to modification of the existing valvular structure. The delivery assembly 1500 can include a prosthetic heart valve 1502 and a delivery system 1510. The prosthetic valve 1502 can be configured to replace a native heart valve (e.g., aortic valve) or previously-implanted prosthetic heart valve. As shown, the prosthetic valve 1502 is releasably coupled to a distal end portion of the delivery system 1510. The delivery system 1510 can be used to deliver and implant the prosthetic valve 1502 in the native heart valve of a subject (see, e.g., FIGS. 16A-16F). The prosthetic valve 1502 can be expandable from a compressed state (e.g., FIG. 15D), for introduction into the existing heart valve, to a fully-expanded state (e.g., FIGS. 15B-15C), for mounting within the existing heart valve. In particular, the prosthetic valve 1502 may have one or more incremental partially-expanded states between the compressed state and the fully-expanded state. The prosthetic valve 1502 can be constructed with one or more locking mechanisms that allow the valve 1502 to maintain a partially-expanded and/or fully-expanded states, for example, to allow positioning of leaflets of the existing heart valve for cutting, as referenced above and in described in further detail below.

FIG. 15B shows a detailed perspective view of the prosthetic valve 1502. As illustrated, the prosthetic valve 1502 comprises three main components: a frame 1532, a valvular structure 1534, and one or more actuators 1536 (e.g., three actuators in the illustrated example). The frame 1532 (which is also referred to herein as “a stent” or “a support structure”) can be constructed to support the valvular structure 1534 and to secure the prosthetic valve 1502 within the existing heart valve. The valvular structure 1534 is coupled to the frame 1532 and/or to the actuators 1536. The valvular structure 1534 is configured to allow blood flow through the prosthetic valve 1502 in one direction (i.e., antegrade) and to restrict blood flow through the prosthetic valve 1502 in the opposite direction (i.e., retrograde). The actuators 1536 can be coupled to the frame 1532 and can be configured to adjust expansion of the frame 1532 between the various states (e.g., compressed, fully-expanded, and one or more partially-expanded states). Note that the valvular structure 1534 of the prosthetic valve 1502 is not shown in FIGS. 15A and 15C-15D in order to assist in the description and illustration of the underlying features.

Referring to FIG. 15C, the frame 1532 of the prosthetic valve 1502 has a first end 1538 and a second end 1540. In the illustrated embodiment, the first end 1538 of the frame 1532 is an inflow end and the second end 1540 of the frame 1532 is an outflow end. In other embodiments, the first end 1538 of the frame 1532 can be the outflow end and the second end 1540 of the frame 1532 can be the inflow end. The frame 1532 can be made of any of various suitable materials, including biocompatible metals and/or biocompatible polymers. Exemplary biocompatible metals from which the frame can be formed include stainless steel, cobalt chromium alloy, and/or nickel titanium alloy.

The frame 1532 can include a plurality of interconnected struts 1542 arranged in a lattice-type pattern. In FIG. 15C, the frame 1532 of the prosthetic valve 1502 is in a radially expanded configuration, which results in the struts 1542 of the frame 1532 extending diagonally relative to a longitudinal axis of the prosthetic valve 1502. In FIG. 15D, the frame 1532 of the prosthetic valve 1502 is in a radially compressed configuration, which results in the struts 1542 of the frame 1532 extending parallel (or at least substantially parallel) to the longitudinal axis of the prosthetic valve 1502. In other configurations (e.g., one or more partially-expanded states), the struts 1542 of the frame 1532 can positions (e.g., offset with respect to adjacent struts) and/or orientations (e.g., angle with respect to the longitudinal axis) different than those depicted in FIGS. 15C-15D.

To transition the valve 1502 between the various states, the struts 1542 of the frame 1532 are pivotably coupled to one another at one or more pivot joints along the length of each strut. For example, each of the struts 1542 can be formed with apertures at opposing ends and along the length of the strut. The frame 1532 can have hinges at locations where struts 1542 overlap. For example, the struts 1542 can be pivotably coupled together via fasteners, such as rivets or pins 1544, that extend through the apertures of the struts 1542. The hinges allow the struts 1542 to pivot relative to one another as the frame 1532 moves between the radially expanded and the radially compressed states, such as during assembly, preparation, delivery, and/or implantation of the prosthetic valve 1502. The frame 1532 can be constructed by forming individual components (e.g., the struts 1542 and pins 1544 of the frame 1532) and then mechanically assembling and coupling the individual components together. Alternatively, the struts are not coupled to each other with respective hinges but are otherwise pivotable or bendable relative to each other to permit radial expansion and contraction of the frame. For example, a frame can be formed (e.g., via laser cutting, electroforming or physical vapor deposition) from a single piece of material (e.g., a metal tube).

Referring again to FIG. 15B, the valvular structure 1534 of the prosthetic valve 1502 is coupled to the frame 1532. The valvular structure 1534 can be configured to allow blood flow through the prosthetic valve 1502 from the inflow end 1538 to the outflow end 1540 and to restrict blood through the prosthetic valve 1502 from the outflow end 1540 to the inflow end 1538. The valvular structure 1534 can include, for example, a leaflet assembly comprising one or more leaflets 1546 (e.g., three leaflets in the illustrated embodiment). As with the other examples described above, leaflets 1546 of the prosthetic valve 1502 can be made of a flexible material, for example, biological material (e.g., pericardium from cow or other sources), bio-compatible synthetic materials, and/or other such materials. The leaflets 1546 can be arranged to form commissures 1548 (e.g., pairs of adjacent leaflets), which can, for example, be mounted to respective actuators 1536. The commissures 1548 of the valve structure 1534 can be coupled to housing members 1552 of the actuators 1536 (see FIGS. 15E-15F), such that the valvular structure 1534 is supported on the frame 1532 by the commissure-actuator couplings. Additional details regarding coupling the valvular structure to the actuators can be found, for example, in International Publication No. WO/2021/003167, which is incorporated herein by reference.

The actuators 1536 of the prosthetic valve 1502 can be mounted to and spaced circumferentially around a radially-inner surface of the frame 1532. The actuators 1536 can be configured to, among other things, radially expand and/or radially compress the frame 1532. Thus, the actuators 1536 can be referred to as “expansion mechanisms.” The actuators 1536 may also be configured to lock the frame 1532 at a desired state (e.g., fully-expanded state, one or more partially-expanded states, etc.). Accordingly, the actuators 1536 can also be referred to as “lockers” or “locking mechanisms.” Each of the actuators 1536 can be configured to form a releasable connection with one or more respective sleeves 1508 of delivery system 1510.

As shown in FIGS. 15E-15F, each actuator 1536 can have a rack member 1550 (which can also be referred to as an “actuation member”), a housing member 1552 (which can also be referred to as a “support member”), and a locking member 1554. The rack members 1550 can be coupled to the frame 1532 of the prosthetic valve 1502 at a first axial location (e.g., closer to the inflow end 1538 of the frame 1532), and the housing members 1552 can be coupled to the frame at a second axial location (e.g., closer to the outflow end 1540 of the frame 1532). The rack members 1550 can extend through and be axially movable relative to respective housing members 1552. Thus, relative axial movement between the rack members 1550 and the housing members 1552 applies axial directed forces to the frame 1532 and results in radial expansion or compression of the frame 1532 as the struts 1542 of the frame 1532 pivot relative to each other about pins 1544. Moving the rack members 1550 proximally (e.g., up in the orientation depicted in FIGS. 15E-15F) relative to the housing members 1552 radially expands the frame 1532 (e.g., toward the state shown in FIGS. 15B-15C). Conversely, moving the rack members 1550 distally (e.g., down in the orientation depicted in FIGS. 15E-15F) relative to the housing members 1552 radially compresses the frame 1532 (e.g., toward the state shown in FIG. 15D).

As shown in FIG. 15F, one or more of the rack members 1550 can include a segment with one or more teeth 1556. Each locking member 1554 can be coupled to a respective housing member 1552 and can comprise one or more pawls 1558 that are biased to engage the teeth 1556 of the rack member 1550. In this manner, the rack member 1550 and the locking member 1554 form a ratchet-type mechanism that allows the rack member 1550 to move proximally relative to the housing member 1552 (thereby allowing expansion of the prosthetic valve 1502) and that restricts the rack member 1550 from moving distally relative to the housing member 1552 (thereby restricting compression of the prosthetic valve 1502).

In the illustrated example, the locking member 1554 is integrally formed with the housing member 1552 as a unitary structure. Alternatively, the locking member 1554 and the housing member 1552 can be formed as separate components that are coupled together (e.g., with fasteners, adhesive, welding, and/or other means for coupling). In the illustrated example, the prosthetic valve 1502 includes three actuators 1536. However, additional or fewer actuators are also possible according to one or more contemplated embodiments. For example, the prosthetic valve can have any number of actuators between one and fifteen, inclusive. Although not shown, the prosthetic valve 1502 can also include one or more skirts or sealing members, for example, as described above for valve 10 in FIGS. 1A-1B. Further details regarding construction and operation of the prosthetic valve 1502 and the delivery system 1510 can be found in International Application Nos. PCT/US2020/057691 and

PCT/US2020/063104, and U.S. Provisional Application No. 62/990,299, each of which is incorporated herein by reference, as well as the other documents incorporated herein by reference above.

As noted above, the prosthetic valve 1502 can be expanded from an initial compressed state (e.g., as illustrated in FIG. 15D) to one or more partially-expanded states within the existing valvular structure and be locked in that state (e.g., via locking member 1554) so as to position leaflets of the existing valvular structure for modification, for example, by cutting, slicing, tearing, or piercing the leaflets or commissures of the existing valvular structure. For example, for introduction into a subject, the prosthetic valve 1502 can be releasably coupled to the delivery system 1510 and can be radially compressed by actuating the actuators 1536, by tensioning a circumferentially-extending recompression member via recompression shaft 1512, and/or by inserting the prosthetic valve 1502 and delivery system 1510 into a crimping device. A first shaft 1504 of the delivery system 1510 can be advanced over a second shaft 1506 of the delivery system 1510 and the prosthetic valve 1502. The compressed prosthetic valve 1502 can thus be disposed within a lumen of the first shaft 1504 and a distal end of the first shaft 1504 can abut a nosecone 1516. A distal end portion of the delivery assembly 1500 can then be inserted into a subject's vasculature, and the prosthetic valve 1502 can be advanced to an implantation location using the delivery system 1510, as shown in FIG. 16A. Note that the illustrated procedure is with respect to a transfemoral delivery of the prosthetic valve; however, other delivery procedures are also possible according to one or more contemplated embodiments, such as transventricular, transapical, transseptal, etc.

In FIG. 16A, the distal end portion of the delivery assembly 1500 is inserted into the subject's vasculature such that the first shaft 1504 extends through the ascending aorta 20 and such that the nosecone 1516 extends through the existing valvular structure 52 (e.g., the annulus of the native aortic valve in FIG. 16A) and into the left ventricle 26 of the subject's heart 50. A guidewire 1560 can be initially extended through the ascending aorta 20 and used to guide and position the distal end portion of the delivery assembly 1500 within a central region of the valvular structure 52 between leaflets thereof. As shown in FIG. 16B, the prosthetic valve 1502 can then be deployed from the first shaft 1504 of the delivery system 1510, for example, by moving the first shaft 1504 proximally relative to the second shaft 1506 and/or by moving the second shaft 1506 distally relative to the first shaft 1504.

The first shaft 1504 can be moved further proximally such that the support sleeves 1508 are exposed from a distal end of the first shaft 1504.

With the prosthetic valve 1502 disposed within the existing valvular structure 52, the valve 1502 can be expanded from the compressed state to a first partially-expanded state, for example, as shown in FIGS. 16C-16D. As noted above, expansion of the frame 1532 of prosthetic valve 1502 can be effected by moving rack member 1550 proximally relative to housing member 1552, for example, via actuation shafts disposed within support sleeves 1508 of the delivery system 1510 (see FIGS. 15A-15F). As the valve 1502 expands, teeth 1556 can displace into engagement with pawl 1558 to thereby lock the frame in each incremental partially-expanded state. As shown in FIG. 16D, the partially-expanded frame within the existing valvular structure 52 creates an annular region between a circumferential exterior of the valve 1502 and the surrounding aortic wall 30, with leaflets 38 of the existing valvular structure 52 captured within the annular region. The annular region is open at its proximal end so as to be accessible from the ascending aorta.

With the partially-expanded frame retaining the leaflets 38 in the annular region, a modification tool 1602 (which may be provided in the ascending aorta 20 by an additional shaft or catheter 1600) can be advanced to the proximal end of the annular region and can be used to modify the existing valvular structure 52 (e.g., by cutting, tearing, slicing, or piercing one or more leaflets 38 or commissures 40). Since the prosthetic valve 1502 is not fully expanded, the leaflets 38 of the existing valvular structure 52 may be sufficiently exposed to the tool 1602 and can enable relatively convenient and efficient modification. In the example illustrated in FIGS. 16C-16D, any of the first through sixteenth exemplary tools described above can be used as modification tool 1602 to modify the existing valvular structure (e.g., the native valve 52 or the valvular structure of an existing, previously implanted prosthetic valve).

In some embodiments, one or more filters 1604 can also be provided. The filter 1604 may be part of the modification tool 1602 or deployed from the same catheter 1600 as the modification tool 1602. Alternatively, the filter 1604 may be part of delivery system 1510 or deployed from the first shaft 1504 or second shaft 1506 of the delivery system 1510. The filter 1604 can extend radially across the ascending aorta proximal of the existing valvular structure 52, for example, so as to contact the aortic wall 30 at an end of filter 1604 (e.g., near or at the sinotubular junction). The filter 1604 can be constructed to allow blood (or at least a portion of the blood) to pass through while capturing particulate or other debris (e.g., portions of the leaflet tissue). For example, if leaflet 38 are highly calcified, the modification can release the calcification into the blood, which may pose a risk to the subject. The filter 1604 can thus capture the debris resulting from the modification of valvular structure 52 by tool 1602. In some embodiments, the filter 1604 can be self-expandable such that it expands to its functional size once deployed from the catheter 1600. The filter 1604 can comprises, for example, a braided structure having a mesh size small enough to trap emboli. Alternatively, the filter 1604 can comprise an expandable membrane (e.g., made of a metal or polymer) formed with perforations or pores small enough to trap emboli.

Once desired modification of the existing valvular structure 52 is completed, the modification tool 1602 (along with its catheter 1600 and optional filter 1604) can be retrieved from the subject, and the prosthetic valve 1502 can be further expanded to the desired fully-expanded state, as shown in FIG. 16E. In some embodiments, the partially-expanded valve 1502 remains coupled to the delivery system 1510 throughout modification of the existing valvular structure 52 by tool 1602. Thus, the delivery system 1510 can be utilized to further expand the frame 1532 from the partially-expanded state to the fully-expanded state, for example, by moving rack member 1550 proximally relative to housing member 1552 using actuation shafts disposed within support sleeves 1508 of the delivery system 1510. As the valve 1502 further expands, other teeth 1556 displace into engagement with pawl 1558 to thereby retain the frame 1532 in the fully-expanded state within the valvular structure 52. Once the prosthetic valve 1502 has been fully expanded and secured, it can then be released from the delivery system 1510, as shown in FIG. 16F. The release can be accomplished by de-coupling the actuation shafts of support sleeves 1508 from the rack members 1550. The support sleeves 1508 and the second shaft 1506 can then be retracted into the first shaft 1504, and the delivery system 1510 can be removed from the subject. Note that for purposes of illustration, the recompression shaft 1512 and recompression member are not shown in FIGS. 16A-16F, and the nosecone 1516 and nosecone shaft 1514 are not shown in FIGS. 16C-16F.

Alternatively, in some embodiments, the delivery system 1510 can be decoupled from the prosthetic heart valve 1502 once the heart valve 1502 is set in the partially-expanded state. For example, the delivery system 1510 can be decoupled from the prosthetic heart valve 1502 and removed from the ascending aorta in order to accommodate the second catheter 1600 of the modification tool 1602 or to deploy a particulate filter proximal of the modification tool 1602. Alternatively, the delivery system 1510 can be decoupled from the prosthetic heart valve 1502 and used to deliver the modification tool 1602 to the existing valvular structure, in which case first shaft 1504 or second shaft 1506 may be considered catheter 1600 of the modification tool 1602. Once desired modification of the existing valvular structure 52 is completed, the modification tool 1602 can be retrieved from the subject, and the prosthetic valve 1502 can be further expanded to the desired fully-expanded state. Since the delivery system 1510 has been decoupled from the valve 1502, further expansion of the valve 1502 may be effected by a separate expansion device disposed within the valve 1502 after the modification. For example, a balloon catheter in a collapsed state can be advanced into a central lumen of the partially-expanded valve 1502, and the balloon catheter can then be inflated to push the valve frame 1532 radially outward to the fully-expanded state. As the valve 1502 further expands, teeth 1556 displace into engagement with pawl 1558 to thereby retain the frame 1532 in the fully-expanded state within the valvular structure 52. Once the prosthetic valve 1502 has been fully expanded and secured, the balloon catheter can be deflated and removed from the subject.

The modification can cause particulate or other debris to be released from the existing valvular structure. For example, if the leaflets of the existing valvular structure are calcified, the cutting or other modification of the leaflets can release the calcification or other particles (e.g., pieces of the leaflet tissue) into the bloodstream. Such debris in the blood stream can pose a risk to the subject, for example, for vascular occlusion or stroke. Thus, in any of the above noted examples, the modification of the existing valvular structure by any of the first through sixteenth tools can include providing one or more filters (such as filter 1604 in FIG. 16D) to capture any particulate or other debris released during the modification. The filter can extend radially across the ascending aorta proximal of the existing valvular structure, for example, to contact the aortic wall (e.g., near or at the sinotubular junction).

For example, the filter (or combination of separate filters) can define a capture region that covers an entire cross-sectional area (or at least a majority of the area, and preferably most of the area) of the blood vessel in the region proximal to the existing valvular structure. In particular, the filter can be constructed to allow blood (or at least a portion of the blood) to pass through while preventing passage of particulate or other debris. For example, the filter can comprise a polymer membrane having a plurality of pores (e.g., each pore having a diameter of ˜100 μm) and supported in an expanded configuration to contact the aortic wall by one or more struts (e.g., formed of a shape-memory alloy). In some embodiments, the filter can be provided as part of the first through sixteenth tools, for example, being deployed from the same catheter or delivery system. In other embodiments, the filter can be provided by a catheter or delivery system separate from that of the first through sixteenth tools.

In still other embodiments, any of the first through sixteenth tools can be used to modify the native valvular structure of another of the native heart valves, for example, pulmonary, tricuspid, or mitral valves, or prosthetic heart valves previously implanted therein. Any of the first through sixteenth tools can thus be configured to be delivered via other blood vessels to the heart. For example, to access the tricuspid or pulmonary positions, the tool(s) can be delivered via the inferior and superior vena cava. For accessing the mitral position, the tool(s) can be delivered via a transseptal procedure, for example, by advancing through the inferior or superior vena cava and across through the atrial septum. Alternatively, for accessing the mitral position, the tool(s) can be delivered via a transapical approach, for example, by advancing through the wall of the left ventricle at the base of the heart.

In any of the disclosed examples, any of the first through sixteenth tools can be provided from the ascending aorta to the existing valvular structure via any transcatheter aortic access route, such as but not limited to transfemoral, transaxillary, transaortic, transapical, transcarotid, transseptal, transcaval, subclavian, radial, or carotid.

Where not otherwise expressly noted above, components of the disclosed tools can be formed of any type of biocompatible material of sufficient strength or flexibility for use in the particular application. Such materials can include, but are not limited to, metals or metal alloys such as surgical steel, titanium, and nickel-titanium (e.g., Nitinol), and polymers such as polyurethane, polytetrafluorethylene, and polyethersulfone.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A heart valve implantation method comprising:

providing an end of a catheter in an ascending aorta of a subject;

extending a first spacing member from the end of the catheter to contact a leaflet of an existing valvular structure between the ascending aorta and a left ventricle of a heart, and extending a second spacing member from the end of the catheter to contact a structure opposite the leaflet along a radial direction, the contact with the second spacing member urging the first spacing member into alignment with the leaflet, the contact with the first spacing member pushing a portion of the leaflet inward with respect to the radial direction;

extending a lacerating member from the end of the catheter to contact the leaflet in a region between a free end of the leaflet and the first spacing member;

cutting the leaflet with the lacerating member, the cutting including retracting the lacerating member in a proximal direction along the leaflet to form a gash that splays the leaflet;

retracting the lacerating member, the first spacing member, and the second spacing member into the catheter, and removing the catheter from the ascending aorta; and

mounting a prosthetic heart valve within the existing valvular structure.

Example 2. The heart valve implantation method of any example herein, particularly Example 1, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 3. The heart valve implantation method of any example herein, particularly Example 2, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 4. The heart valve implantation method of any example herein, particularly any one of Examples 2-3, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 5. The heart valve implantation method of any example herein, particularly any one of Examples 2-3, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 6. The heart valve implantation method of any example herein, particularly any one of Examples 1-5, further comprising capturing particulate or debris released from the existing valvular structure by the cutting of the leaflet.

Example 7. The heart valve implantation method of any example herein, particularly Example 6, wherein the capturing is by a filter extended from the end of the catheter and disposed proximal of the existing valvular structure.

Example 8. The heart valve implantation method of any example herein, particularly any one of Examples 1-7, wherein the cutting with the lacerating member comprises applying electrical energy to the lacerating member.

Example 9. The heart valve implantation method of any example herein, particularly any one of Examples 1-8, wherein the lacerating member extending from the end of the catheter has a hook-shape with a sharp tip, and the cutting includes piercing the leaflet with the sharp tip.

Example 10. The heart valve implantation method of any example herein, particularly any one of Examples 1-9, wherein the first spacing member, the second spacing member, or the lacerating member comprises a wire formed of a shape-memory alloy.

Example 11. The heart valve implantation method of any example herein, particularly any one of Examples 1-10, wherein the first spacing member extending from the end of the catheter has a circular or elliptical shape, and/or the second spacing member extending from the end of the catheter has a bend or is curved along its length.

Example 12. The heart valve implantation method of any example herein, particularly any one of Examples 1-11, wherein the first spacing member comprises a pair of wires coupled to each other at respective proximal ends.

Example 13. The heart valve implantation method of any example herein, particularly any one of Examples 1-12, further comprising:

after the cutting and before the removing the catheter, rotating the first and second spacing members and the lacerating member within the ascending aorta; and

repeating the extending the first spacing member, the extending the second spacing member, the extending the lacerating member, and the cutting for a different leaflet of the valvular structure.

Example 14. The heart valve implantation method of any example herein, particularly any one of Examples 1-13, wherein:

the end of the catheter has a multi-channel positioning member with channels through which the first spacing member, the second spacing member, and the lacerating member respectively extend, and

the rotating comprises rotating the positioning member via a shaft coupled thereto, the positioning member maintaining relative positions between the first spacing member, the second spacing member, and the lacerating member during the rotating.

Example 15. The heart valve implantation method of any example herein, particularly Example 14, wherein the positioning member has a conical or tapered shape.

Example 16. The heart valve implantation method of any example herein, particularly any one of Examples 1-15, wherein the existing valvular structure is a native aortic valve of the heart, and the structure contacted by the second spacing member is a wall of the ascending aorta.

Example 17. The heart valve implantation method of any example herein, particularly Example 16, wherein the gash in the splayed leaflet allows blood to flow through a frame of the prosthetic heart valve to a coronary artery of the heart, which would otherwise be blocked by the uncut leaflet of the native aortic valve.

Example 18. The heart valve implantation method of any example herein, particularly any one of Examples 16-17, wherein the native aortic valve is a bicuspid aortic valve having two leaflets, the prosthetic heart valve has a cylindrical shape, and the gash in the splayed leaflet allows the prosthetic heart valve to be mounted within the bicuspid aortic valve.

Example 19. The heart valve implantation method of any example herein, particularly any one of Examples 1-15, wherein the existing valvular structure is of a second prosthetic valve previously implanted in the subject, the structure contacted by the second spacing member is a frame of the second prosthetic valve, and the mounting comprises installing the prosthetic heart valve within the second prosthetic valve.

Example 20. The heart valve implantation method of any example herein, particularly Example 19, wherein the gash in the splayed leaflet allows blood to flow through a frame of the prosthetic heart valve en route to a coronary artery of the heart, which would otherwise be blocked by the uncut leaflet of the previously-implanted second prosthetic valve.

Example 21. A cutting tool for modifying a valvular structure comprising:

a catheter having an end configured to be disposed within an ascending aorta of a subject;

a first spacing member within the catheter and configured to be extended from the end of the catheter to contact a leaflet of an existing valvular structure;

a second spacing member within the catheter and configured to be extended from the end of the catheter to contact a structure opposite the leaflet along a radial direction; and

a lacerating member within the catheter and configured to be extended from the end of the catheter to contact and cut the leaflet in a region between a free end of the leaflet and the first spacing member.

Example 22. The cutting tool of any example herein, particularly Example 21, wherein the lacerating member is configured to cut using electrical energy applied thereto.

Example 23. The cutting tool of any example herein, particularly any one of Examples 21-22, wherein the lacerating member extending from the end of the catheter has a hook-shape with a sharp tip.

Example 24. The cutting tool of any example herein, particularly any one of Examples 21-23, wherein the first spacing member, the second spacing member, or the lacerating member comprises a wire formed of a shape-memory alloy.

Example 25. The cutting tool of any example herein, particularly any one of Examples 21-24, wherein the first spacing member extending from the end of the catheter has a circular or elliptical shape, and/or the second spacing member extending from the end of the catheter has a bend or is curved along its length.

Example 26. The cutting tool of any example herein, particularly any one of Examples 21-25, wherein the first spacing member comprises a pair of wires coupled to each other at respective proximal ends.

Example 27. The cutting tool of any example herein, particularly any one of Examples 21-26, further comprising one or more filters within the catheter and configured to be extended from the end of the catheter, each filter being constructed to capture particulate or other debris released by cutting of the leaflet.

Example 28. The cutting tool of any example herein, particularly Example 27, wherein each filter is configured to be extended radially from the end of the catheter to contact a wall of the aorta proximal of the existing valvular structure.

Example 29. The cutting tool of any example herein, particularly any one of Examples 21-28, wherein the end of the catheter has a multi-channel positioning member with channels through which the first spacing member, the second spacing member, and the lacerating member respectively extend, and the positioning member is configured to maintain relative positions between the first spacing member, the second spacing member, and the lacerating member during the rotation of the positioning member within the ascending aorta.

Example 30. A heart valve implantation method comprising:

providing an end of a catheter in an ascending aorta of a subject, a head portion being coupled to the end of the catheter, a wall of the head portion having a first recess and a second recess on an opposite side from the first recess, the first recess having a cutting element at an edge thereof;

cutting a commissure of adjacent leaflets of an existing valvular structure by inserting the commissure into the first and second recesses of the head portion, the second recess gripping the inserted commissure while the cutting element of the first recess cuts the inserted commissure, the existing valvular structure being between the ascending aorta and a left ventricle of a heart, the cut commissure freeing the adjacent leaflets to collapse distally toward the left ventricle;

removing the catheter from the ascending aorta; and

mounting a prosthetic heart valve within the existing valvular structure.

Example 31. The heart valve implantation method of any example herein, particularly Example 30, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 32. The heart valve implantation method of any example herein, particularly Example 31, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 33. The heart valve implantation method of any example herein, particularly any one of Examples 32-33, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 34. The heart valve implantation method of any example herein, particularly any one of Examples 32-33, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 35. The heart valve implantation method of any example herein, particularly any one of Examples 30-34, further comprising, capturing particulate or debris released from the existing valvular structure by the cutting of the commissure.

Example 36. The heart valve implantation method of any example herein, particularly Example 35, wherein the capturing is by a filter extended from the end of the catheter and disposed proximal of the existing valvular structure.

Example 37. The heart valve implantation method of any example herein, particularly any one of Examples 30-36, wherein the first recess or the second recess has a U-shape with a curved proximal edge, an A-shape with a narrow proximal edge, or rectangular shape with a straight proximal edge.

Example 38. The heart valve implantation method of any example herein, particularly any one of Examples 30-37, wherein the providing comprises extending a guidewire through a lumen of the catheter toward the existing valvular structure, wherein the guidewire orients itself within the commissure.

Example 39. The heart valve implantation method of any example herein, particularly any one of Examples 30-38, further comprising:

after the cutting and before the removing the catheter, repositioning the head portion with respect to another commissure; and

repeating the cutting for the another commissure of the existing valvular structure.

Example 40. The heart valve implantation method of any example herein, particularly any one of Examples 30-39, wherein the cutting comprises applying electrical energy to the cutting element.

Example 41. The heart valve implantation method of any example herein, particularly any one of Examples 30-40, wherein the cutting element comprises a sharp edge of the first recess or a separate blade.

Example 42. The heart valve implantation method of any example herein, particularly any one of Examples 30-41, wherein the existing valvular structure is a native aortic valve of the heart.

Example 43. The heart valve implantation method of any example herein, particularly Example 42, wherein the collapsed leaflets allow blood to flow through a frame of the prosthetic heart valve to a coronary artery of the heart, which would otherwise be blocked by the leaflets of the native aortic valve.

Example 44. The heart valve implantation method of any example herein, particularly any one of Examples 42-43, wherein the native aortic valve is a bicuspid aortic valve having two leaflets, the prosthetic heart valve has a cylindrical shape, and the collapsed leaflets allow the prosthetic heart valve to be mounted within the bicuspid aortic valve.

Example 45. The heart valve implantation method of any example herein, particularly any one of Examples 30-41, wherein the existing valvular structure is of a second prosthetic valve previously implanted in the subject, and the mounting comprises installing the prosthetic heart valve within the second prosthetic valve.

Example 46. The heart valve implantation method of any example herein, particularly Example 45, wherein the collapsed leaflets allow blood to flow through a frame of the prosthetic heart valve en route to a coronary artery of the heart, which would otherwise be blocked by the leaflets of the previously-implanted second prosthetic valve.

Example 47. A cutting tool for modifying a valvular structure comprising:

a catheter having an end configured to be disposed within an ascending aorta of a subject; and

a head portion coupled to the end of the catheter,

wherein the head portion has a wall with a first recess and a second recess on an opposite side from the first recess,

the first recess has a cutting element at an edge thereof and being configured to cut a commissure of an existing valvular structure inserted into the first and second recesses, and

the second recess is configured to grip the commissure inserted into the first and second recesses.

Example 48. The cutting tool of any example herein, particularly Example 47, wherein the first recess or the second recess has a U-shape with a curved proximal edge, an A-shape with a narrow proximal edge, or a rectangular shape with a straight proximal edge.

Example 49. The cutting tool of any example herein, particularly any one of Examples 47-48, wherein a shape of the first recess is different than a shape of the second recess.

Example 50. The cutting tool of any example herein, particularly any one of Examples 47-49, further comprising a guidewire that extends through a lumen of the catheter and is configured to orient itself within the commissure.

Example 51. The cutting tool of any example herein, particularly any one of Examples 47-50, wherein the cutting element is configured to cut using electrical energy applied thereto.

Example 52. The cutting tool of any example herein, particularly any one of Examples 47-51, wherein the cutting element comprises a sharp edge of the first recess or a separate blade.

Example 53. The cutting tool of any example herein, particularly any one of Examples 47-52, further comprising one or more filters constructed to capture particulate or other debris released by cutting of the commissure.

Example 54. The cutting tool of any example herein, particularly Example 53, wherein each filter is configured to extend radially from a catheter to contact a wall of the aorta proximal of an existing valvular structure.

Example 55. A heart valve implantation method comprising:

providing an end of a catheter in an ascending aorta of a subject and arranged with respect to a commissure of adjacent leaflets of an existing valvular structure, the existing valvular structure being between the ascending aorta and a left ventricle of a heart;

extending a gripping mechanism from the end of the catheter such that the commissure is disposed within a first recess formed by first and second arms of the gripping mechanism;

gripping the commissure by moving at least one of the first and second arms with respect to the other such that the first and second arms contact opposite sides of the commissure;

extending a cutting mechanism from the end of the catheter such that the commissure is disposed within a second recess formed by third and fourth arms of the cutting mechanism;

cutting the commissure by moving at least one of the third and fourth arms with respect to the other such that the third and fourth arms slice through the commissure, the cut commissure freeing the adjacent leaflets to collapse distally toward the left ventricle;

removing the catheter from the ascending aorta; and

mounting a prosthetic heart valve within the existing valvular structure.

Example 56. The heart valve implantation method of any example herein, particularly Example 55, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 57. The heart valve implantation method of any example herein, particularly Example 56, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 58. The heart valve implantation method of any example herein, particularly any one of Examples 56-57, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 59. The heart valve implantation method of any example herein, particularly any one of Examples 56-57, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 60. The heart valve implantation method of any example herein, particularly any one of Examples 55-59, further comprising, capturing particulate or debris released from the existing valvular structure by the cutting of the commissure.

Example 61. The heart valve implantation method of any example herein, particularly Example 60, wherein the capturing is by a filter extended from the end of the catheter and disposed proximal of the existing valvular structure.

Example 62. The heart valve implantation method of any example herein, particularly any one of Examples 55-61, wherein the extending the gripping mechanism and the extending the cutting mechanism occur simultaneously.

Example 63. The heart valve implantation method of any example herein, particularly any one of Examples 55-61, wherein the gripping the commissure is before the cutting the commissure.

Example 64. The heart valve implantation method of any example herein, particularly any one of Examples 55-63, wherein the first arm and/or the second arm has an edge facing the first recess that is serrated or has a plurality of teeth along its length.

Example 65. The heart valve implantation method of any example herein, particularly any one of Examples 55-64, wherein the third arm and/or the fourth arm has a sharp edge facing the second recess or a blade portion facing the second recess.

Example 66. The heart valve implantation method of any example herein, particularly any one of Examples 55-65, wherein the cutting the commissure comprises applying electrical energy to the third arm and/or the fourth arm.

Example 67. The heart valve implantation method of any example herein, particularly any one of Examples 55-66, wherein the providing comprises extending a guidewire through a lumen of the catheter toward the existing valvular structure, wherein the guidewire orients itself within the commissure.

Example 68. The heart valve implantation method of any example herein, particularly any one of Examples 55-67, further comprising, after the cutting and before the removing the catheter:

moving at least one of the third and fourth arms with respect to the other such that the third and fourth arms open to reform the second recess;

moving at least one of the first and second arms with respect to the other such that the first and second arms open to reform the first recess;

repositioning the end of the catheter with respect to another commissure; and

repeating the gripping and cutting for the another commissure of the existing valvular structure.

Example 69. The heart valve implantation method of any example herein, particularly any one of Examples 55-68, wherein a length of the first arm or the second arm is less than a length of the third arm or the fourth arm, and the length of the third arm or the fourth arm is greater than a length of the commissure within the second recess.

Example 70. The heart valve implantation method of any example herein, particularly any one of Examples 55-69, further comprising, after the cutting:

moving at least one of the third and fourth arms with respect to the other such that the third and fourth arms open to reform the second recess; and

withdrawing the gripping mechanism toward the catheter while the first and second arms grip the cut portion of the commissure so as to retract or remove the cut portion of the commissure.

Example 71. The heart valve implantation method of any example herein, particularly any one of Examples 55-70, wherein the existing valvular structure is a native aortic valve of the heart.

Example 72. The heart valve implantation method of any example herein, particularly Example 71, wherein the collapsed leaflets allow blood to flow through a frame of the prosthetic heart valve to a coronary artery of the heart, which would otherwise be blocked by the leaflets of the native aortic valve.

Example 73. The heart valve implantation method of any example herein, particularly any one of Examples 71-72, wherein the native aortic valve is a bicuspid aortic valve having two leaflets, the prosthetic heart valve has a cylindrical shape, and the collapsed leaflets allow the prosthetic heart valve to be mounted within the bicuspid aortic valve.

Example 74. The heart valve implantation method of any example herein, particularly any one of Examples 55-70, wherein the existing valvular structure is of a second prosthetic valve previously implanted in the subject, and the mounting comprises installing the prosthetic heart valve within the second prosthetic valve.

Example 75. The heart valve implantation method of any example herein, particularly Example 74, wherein the collapsed leaflets allow blood to flow through a frame of the prosthetic heart valve to a coronary artery of the heart, which would otherwise be blocked by the leaflets of the previously-implanted second prosthetic valve.

Example 76. A cutting tool for modifying a valvular structure comprising:

a catheter having an end configured to be disposed within an ascending aorta of a subject;

a gripping mechanism within the catheter and configured to be extended from the end of the catheter, the gripping mechanism having first and second arms that form a first recess, at least one of the first and second arms being movable with respect to the other so as to grip a commissure of an existing valvular structure disposed within the first recess; and

a cutting mechanism within the catheter and configured to be extended from the end of the catheter, the cutting mechanism having third and fourth arms that form a second recess, at least one of the third and fourth arms being movable with respect to the other so as to cut the commissure disposed within the second recess.

Example 77. The cutting tool of any example herein, particularly Example 76, wherein the first arm and/or the second arm has an edge facing the first recess that is serrated or has a plurality of teeth along its length.

Example 78. The cutting tool of any example herein, particularly any one of Examples 76-77, wherein the third arm and/or the fourth arm has a sharp edge facing the second recess or a blade portion facing the second recess.

Example 79. The cutting tool of any example herein, particularly any one of Examples 76-78, wherein the cutting mechanism is configured to cut using electrical energy applied to the third arm and/or the fourth arm.

Example 80. The cutting tool of any example herein, particularly any one of Examples 76-79, further comprising a guidewire that extends through a lumen of the catheter and is configured to orient itself within the commissure.

Example 81. The cutting tool of any example herein, particularly any one of Examples 76-80, wherein a length of the first arm or the second arm is less than a length of the third arm or the fourth arm.

Example 82. The cutting tool of any example herein, particularly any one of Examples 76-81, wherein a first actuation member extends through a lumen of the catheter and is coupled to the gripping mechanism, and a second actuation member extends through the lumen of the catheter and is coupled to the cutting mechanism.

Example 83. The cutting tool of any example herein, particularly Example 82, wherein the first actuation member or the second actuation member comprises a wire or cable.

Example 84. The cutting tool of any example herein, particularly any one of Examples 76-83, further comprising one or more filters constructed to capture particulate or other debris released by cutting of the commissure.

Example 85. The cutting tool of any example herein, particularly Example 84, wherein each filter is configured to be extended radially from the end of the catheter to contact a wall of the aorta proximal of the existing valvular structure.

Example 86. A heart valve implantation method comprising:

providing an end of a catheter in an ascending aorta of a subject;

extending a cutting frame from the end of the catheter, the cutting frame having a plurality of first apices at a proximal end and a plurality of second apices at a distal end, each first apex being connected to a pair of the second apices by respective struts and having a respective cutting element, the extending being such that the cutting frame expands from a first diameter within the catheter to a second diameter greater than the first diameter outside the catheter;

positioning each of the first apices into contact with a corresponding commissure of adjacent leaflets of an existing valvular structure, the existing valvular structure being between the ascending aorta and a left ventricle of a heart;

cutting each commissure by moving the cutting frame distally such that the cutting elements of the first apices slice through the commissures, the cut commissure freeing the adjacent leaflets to collapse distally toward the left ventricle;

removing the catheter from the ascending aorta; and

mounting a prosthetic heart valve within the existing valvular structure.

Example 87. The heart valve implantation method of any example herein, particularly Example 86, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 88. The heart valve implantation method of any example herein, particularly Example 87, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 89. The heart valve implantation method of any example herein, particularly any one of Examples 87-88, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 90. The heart valve implantation method of any example herein, particularly any one of Examples 87-88, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 91. The heart valve implantation method of any example herein, particularly any one of Examples 86-90, further comprising, capturing particulate or debris released from the existing valvular structure by the cutting each commissure.

Example 92. The heart valve implantation method of any example herein, particularly Example 91, wherein the capturing is by a filter extended from the end of the catheter and disposed proximal of the existing valvular structure.

Example 93. The heart valve implantation method of any example herein, particularly any one of Examples 86-92, wherein the second apices are configured to automatically align the cutting frame with respect to the existing valvular structure during the positioning each of the first apices.

Example 94. The heart valve implantation method of any example herein, particularly any one of Examples 86-93, wherein the cutting element of each first apex comprises a sharp edge of the first apex or a separate blade.

Example 95. The heart valve implantation method of any example herein, particularly any one of Examples 86-94, wherein the cutting comprises applying electrical energy to the cutting elements of the first apices of the cutting frame.

Example 96. The heart valve implantation method of any example herein, particularly any one of Examples 86-95, further comprising, prior to removing the catheter:

contracting the cutting frame to a third diameter less than the second diameter; and

retracting the contracted cutting frame into the catheter.

Example 97. The heart valve implantation method of any example herein, particularly any one of Examples 86-96, wherein each second apex is connected to a respective support arm extending from the end of the catheter.

Example 98. The heart valve implantation method of any example herein, particularly Example 97, further comprising, prior to the removing the catheter, retracting the support arms such that the second apices of the cutting frame are pulled into the end of the catheter before the first apices of the cutting frame, the retracting being such that the cutting frame inverts and adopts a third diameter less than the second diameter.

Example 99. The heart valve implantation method of any example herein, particularly any one of Examples 86-98, wherein the cutting frame and/or the support arms is formed of a shape-memory alloy.

Example 100. The heart valve implantation method of any example herein, particularly any one of Examples 86-99, wherein the existing valvular structure is a native aortic valve of the heart.

Example 101. The heart valve implantation method of any example herein, particularly Example 100, wherein the collapsed leaflets allow blood to flow through a frame of the prosthetic heart valve to a coronary artery of the heart, which would otherwise be blocked by the leaflets of the native aortic valve.

Example 102. The heart valve implantation method of any example herein, particularly any one of Examples 100-101, wherein the native aortic valve is a bicuspid aortic valve having two leaflets, the prosthetic heart valve has a cylindrical shape, and the collapsed leaflets allow the prosthetic heart valve to be mounted within the bicuspid aortic valve.

Example 103. The heart valve implantation method of any example herein, particularly any one of Examples 86-99, wherein the existing valvular structure is of a second prosthetic valve previously implanted in the subject, and the mounting comprises installing the prosthetic heart valve within the second prosthetic valve.

Example 104. The heart valve implantation method of any example herein, particularly Example 103, wherein the collapsed leaflets allow blood to flow through a frame of the prosthetic heart valve en route to a coronary artery of the heart, which would otherwise be blocked by the leaflets of the previously-implanted second prosthetic valve.

Example 105. A cutting tool for modifying a valvular structure comprising:

a catheter having an end configured to be disposed within an ascending aorta of a subject; and

a cutting frame movably disposed within the catheter, the cutting frame having a plurality of first apices at a proximal end and a plurality of second apices at a distal end, each first apex being connected to a pair of the second apices by respective struts, the cutting frame being configured to expand from a first diameter within the catheter to a second diameter greater than the first diameter outside the catheter,

wherein the second apices are configured to position the cutting frame with respect to an existing valvular structure such that commissures of the valvular structure are respectively contacted with the first apices, and

each first apex has a cutting element configured to slice through the respective commissure.

Example 106. The cutting tool of any example herein, particularly Example 105, further comprising a plurality of support arms extending through a lumen of the catheter, each of the support arms being connected to a respective one of the second apices.

Example 107. The cutting tool of any example herein, particularly any one of Examples 105-106, wherein the cutting frame and/or the support arms is formed of a shape-memory alloy.

Example 108. The cutting tool of any example herein, particularly any one of Examples 105-107, wherein the cutting element of each first apex comprises a sharp edge of the first apex or a separate blade.

Example 109. The cutting tool of any example herein, particularly any one of Examples 105-108, wherein the cutting elements are configured to slice using electrical energy applied thereto.

Example 110. The cutting tool of any example herein, particularly any one of Examples 105-109, wherein in an expanded state of the cutting frame having the second diameter, the second apices are disposed radially inward with respect to the first apices.

Example 111. The cutting tool of any example herein, particularly any one of Examples 105-110, further comprising one or more filters within the catheter and configured to be extended from the end of the catheter, each filter being constructed to capture particulate or other debris released by cutting of the commissures.

Example 112. The cutting tool of any example herein, particularly Example 111, wherein each filter is configured to be extended radially from the end of the catheter to contact a wall of the aorta proximal of the existing valvular structure.

Example 113. The cutting tool of any example herein, particularly any one of Examples 105-110, wherein the cutting frame includes one or more filters constructed to capture particulate or other debris released by cutting of the commissures.

Example 114. A heart valve implantation method comprising:

providing an end of a catheter in an ascending aorta of a subject;

extending a leaflet cutting device from the end of the catheter, the leaflet cutting device comprising an expansion device, a positioning member, and a cutting element between the expansion device and the positioning member, the expansion device being expandable from a first diameter to a second diameter greater than the first diameter, the extending being such that the expansion device is centrally located between leaflets of an existing valvular structure and such that a portion of the positioning member is on a radially outer side of one of the leaflets, the existing valvular structure being between the ascending aorta and a left ventricle of a heart;

expanding the expansion device to the second diameter such that the cutting element is urged toward the positioning member, thereby cutting said one of the leaflets between the cutting element and the positioning member;

removing the catheter from the ascending aorta; and

mounting a prosthetic heart valve within the existing valvular structure.

Example 115. The heart valve implantation method of any example herein, particularly Example 114, wherein the expansion device comprises a balloon, a self-expanding frame, or a mechanically-expandable frame.

Example 116. The heart valve implantation method of any example herein, particularly any one of Examples 114-115, further comprising, capturing particulate or debris released from the existing valvular structure by the cutting of the leaflet.

Example 117. The heart valve implantation method of any example herein, particularly Example 116, wherein the capturing is by a filter extended from the end of the catheter and disposed proximal of the existing valvular structure.

Example 118. The heart valve implantation method of any example herein, particularly any one of Examples 114-117, wherein the positioning member has a recess or gap for receiving the cutting element therein to effect the cutting.

Example 119. The heart valve implantation method of any example herein, particularly Example 118, wherein the recess or gap has an inverted U-shape or inverted V-shape with an open distal end, a rectangular shape with an open distal end, a U-shape or V-shape with a closed distal end, a circular shape with a closed distal end, an oval shape with a closed distal end, an elliptical shape with a closed distal end, or a rectangular shape with a closed distal end.

Example 120. The heart valve implantation method of any example herein, particularly any one of Examples 118-119, wherein the cutting element has a shape complementary to that of the recess or gap of the positioning member.

Example 121. The heart valve implantation method of any example herein, particularly any one of Examples 114-120, wherein the cutting element has a sharp edge or comprises a blade.

Example 122. The heart valve implantation method of any example herein, particularly any one of Examples 114-121, wherein the cutting comprises applying electrical energy to the cutting element.

Example 123. The heart valve implantation method of any example herein, particularly any one of Examples 114-122, wherein:

the leaflet cutting device comprises multiple pairs of the positioning member and cutting element, each pair corresponding to one of the leaflets of the existing valvular structure, and

the expanding is such that each leaflet is simultaneously cut by the respective cutting element.

Example 124. The heart valve implantation method of any example herein, particularly any one of Examples 114-123, wherein the positioning member is formed of a shape-memory alloy.

Example 125. The heart valve implantation method of any example herein, particularly any one of Examples 114-124, wherein, by the expanding, the cutting element becomes coupled to the positioning member by a snap-fit feature such that the cutting element is retained by the positioning member despite a decrease in size or removal of the expansion device.

Example 126. The heart valve implantation method of any example herein, particularly any one of Examples 114-125, further comprising, prior to the removing the catheter, contracting the expansion device and retracting the leaflet cutting device into the catheter.

Example 127. The heart valve implantation method of any example herein, particularly Example 126, wherein the retracting pulls cut portions of the leaflet into the catheter via the cutting element connected to the positioning member.

Example 128. The heart valve implantation method of any example herein, particularly any one of Examples 114-127, wherein the existing valvular structure is a native aortic valve of the heart, and the expanding the expansion device simultaneously performs a balloon annular valvuloplasty (BAV) procedure on the native valve or is performed prior to a BAV procedure.

Example 129. The heart valve implantation method of any example herein, particularly Example 128, wherein the cut leaflet allows blood to flow through a frame of the prosthetic heart valve to a coronary artery of the heart, which would otherwise be blocked by the uncut leaflet of the native aortic valve.

Example 130. The heart valve implantation method of any example herein, particularly any one of Examples 128-129, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 131. The heart valve implantation method of any example herein, particularly any one of Examples 114-127, wherein the existing valvular structure is of a second prosthetic valve previously implanted in the subject, and the mounting comprises installing the prosthetic heart valve within the second prosthetic valve.

Example 132. The heart valve implantation method of any example herein, particularly Example 131, wherein the cut leaflet allows blood to flow through a frame of the prosthetic heart valve en route to a coronary artery of the heart, which would otherwise be blocked by the uncut leaflet of the previously-implanted second prosthetic valve.

Example 133. A cutting tool for modifying a valvular structure comprising:

a catheter having an end configured to be disposed within an ascending aorta of a subject;

an expansion device within the catheter and configured to be extended from the end of the catheter to be centrally located between leaflets of an existing valvular structure, the expansion device being expandable from a first diameter to a second diameter greater than the first diameter;

one or more positioning members within the catheter and configured to be extended from the end of the catheter such that a portion of each positioning member is on a radially outer side of a respective one of the leaflets; and

one or more cutting elements within the catheter and configured to be extended from the end of the catheter, each cutting element being between the expansion device and a respective positioning member and being configured to cut said one of the leaflets when urged into contact therewith by expansion of the expansion device.

Example 134. The cutting tool of any example herein, particularly Example 133, wherein the expansion device comprises a balloon, a self-expanding frame, or a mechanically-expandable frame.

Example 135. The cutting tool of any example herein, particularly any one of Examples 133-134, each positioning member has a recess or gap for receiving the respective cutting element therein to effect the cutting.

Example 136. The cutting tool of any example herein, particularly Example 135, wherein the recess or gap has an inverted U-shape or inverted V-shape with an open distal end, a rectangular shape with an open distal end, a U-shape or V-shape with a closed distal end, a circular shape with a closed distal end, an elliptical shape with a closed distal end, or a rectangular shape with a closed distal end.

Example 137. The cutting tool of any example herein, particularly any one of Examples 135-136, wherein each cutting element has a shape complementary to that of the recess or gap of the respective positioning member.

Example 138. The cutting tool of any example herein, particularly any one of Examples 133-137, wherein each cutting element has a sharp edge or comprises a blade.

Example 139. The cutting tool of any example herein, particularly any one of Examples 133-138, wherein each cutting element is configured to cut using electrical energy applied thereto.

Example 140. The cutting tool of any example herein, particularly any one of Examples 133-139, wherein a number of the positioning members and a number of cutting elements is equal to a number of the leaflets of the existing valvular structure.

Example 141. The cutting tool of any example herein, particularly any one of Examples 133-140, wherein the positioning member is formed of a shape-memory alloy.

Example 142. The cutting tool of any example herein, particularly any one of Examples 133-141, wherein each cutting element and/or each positioning member is configured with a snap-fit feature that couples the cutting element to the respective positioning member upon expansion of the expansion device to the second diameter.

Example 143. The cutting tool of any example herein, particularly any one of Examples 133-142, further comprising one or more filters constructed to capture particulate or other debris released by cutting of the leaflet.

Example 144. The cutting tool of any example herein, particularly Example 143, wherein each filter is configured to be extended radially from the end of the catheter to contact a wall of the aorta proximal of the existing valvular structure.

Example 145. A heart valve implantation method comprising:

providing an end of a catheter in an ascending aorta of a subject;

extending a leaflet cutting device from the end of the catheter, the leaflet cutting device comprising a tubular member, a positioning member, and a cutting element, the positioning member and cutting element extending from within a lumen of the tubular member, the extending being such that a leaflet of an existing valvular structure is between the positioning member and the cutting element, the existing valvular structure being between the ascending aorta and a left ventricle of a heart;

cutting the leaflet by axially displacing the tubular member over the positioning member and the cutting element from a proximal position, where the cutting element is spaced from the positioning member, to a distal position, where the cutting element and the positioning member are urged together with the leaflet therebetween,

removing the catheter from the ascending aorta; and

mounting a prosthetic heart valve within the existing valvular structure.

Example 146. The heart valve implantation method of any example herein, particularly Example 145, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 147. The heart valve implantation method of any example herein, particularly Example 146, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 148. The heart valve implantation method of any example herein, particularly any one of Examples 146-147, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 149. The heart valve implantation method of any example herein, particularly any one of Examples 146-147, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 150. The heart valve implantation method of any example herein, particularly any one of Examples 145-149, further comprising, capturing particulate or debris released from the existing valvular structure by the cutting of the leaflet.

Example 151. The heart valve implantation method of any example herein, particularly Example 150, wherein the capturing is by a filter extended from the end of the catheter and disposed proximal of the existing valvular structure.

Example 152. The heart valve implantation method of any example herein, particularly any one of Examples 145-151, wherein a distal end of the tubular member has a diameter greater than a proximal end of the tubular member.

Example 153. The heart valve implantation method of any example herein, particularly any one of Examples 145-152, wherein the positioning member has a recess or gap for receiving the cutting element therein to effect the cutting.

Example 154. The heart valve implantation method of any example herein, particularly Example 153, wherein the recess or gap has an inverted U-shape or inverted V-shape with an open distal end, a rectangular shape with an open distal end, a U-shape or V-shape with a closed distal end, a circular shape with a closed distal end, an elliptical shape with a closed distal end, or a rectangular shape with a closed distal end.

Example 155. The heart valve implantation method of any example herein, particularly any one of Examples 153-154, wherein the cutting element has a shape complementary to that of the recess or gap of the positioning member.

Example 156. The heart valve implantation method of any example herein, particularly any one of Examples 145-155, wherein the cutting element has a sharp edge or comprises a blade.

Example 157. The heart valve implantation method of any example herein, particularly any one of Examples 145-156, wherein the cutting comprises applying electrical energy to the cutting element.

Example 158. The heart valve implantation method of any example herein, particularly any one of Examples 145-157, wherein:

the leaflet cutting device comprises multiple sets of the tubular member, the positioning member, and the cutting element, each set corresponding to one of the leaflets of the existing valvular structure, and

the cutting is such that each leaflet is simultaneously cut by the respective cutting element.

Example 159. The heart valve implantation method of any example herein, particularly any one of Examples 145-158, further comprising:

after the cutting and before the removing the catheter, repositioning the leaflet cutting device within the ascending aorta for a different leaflet of the existing valvular structure; and

repeating the extending and the cutting for the different leaflet.

Example 160. The heart valve implantation method of any example herein, particularly any one of Examples 145-159, wherein the positioning member is formed of a shape-memory alloy.

Example 161. The heart valve implantation method of any example herein, particularly any one of Examples 145-160, wherein, by the displacing the tubular member, the cutting element becomes coupled to the positioning member by a snap-fit feature such that the cutting element is retained by the positioning member regardless of a change in position of the tubular member.

Example 162. The heart valve implantation method of any example herein, particularly any one of Examples 145-161, further comprising, prior to the removing the catheter, retracting the leaflet cutting device into the catheter, wherein the retracting pulls cut portions of the leaflet into the catheter via the cutting element connected to the positioning member.

Example 163. The heart valve implantation method of any example herein, particularly any one of Examples 145-162, wherein the existing valvular structure is a native aortic valve of the heart, and the expanding the expansion device simultaneously performs a balloon annular valvuloplasty (BAV) procedure on the native valve or is performed prior to a BAV procedure.

Example 164. The heart valve implantation method of any example herein, particularly Example 163, wherein the cut leaflet allows blood to flow through a frame of the prosthetic heart valve to a coronary artery of the heart, which would otherwise be blocked by the uncut leaflet of the native aortic valve.

Example 165. The heart valve implantation method of any example herein, particularly any one of Examples 163-164, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 166. The heart valve implantation method of any example herein, particularly any one of Examples 145-162, wherein the existing valvular structure is of a second prosthetic valve previously implanted in the subject, and the mounting comprises installing the prosthetic heart valve within the second prosthetic valve.

Example 167. The heart valve implantation method of any example herein, particularly Example 166, wherein the cut leaflet allows blood to flow through a frame of the prosthetic heart valve en route to a coronary artery of the heart, which would otherwise be blocked by the uncut leaflet of the previously-implanted second prosthetic valve.

Example 168. A cutting tool for modifying a valvular structure comprising:

a catheter having an end configured to be disposed within an ascending aorta of a subject;

a positioning member within the catheter and configured to be extended from the end of the catheter such that a portion of the positioning member is on a first side of a leaflet of an existing valvular structure;

a cutting element within the catheter and configured to be extended from the end of the catheter such that a portion of the cutting element is on a second side of the leaflet; and

a tubular member having a lumen through which the positioning member and the cutting element extend,

wherein the tubular member is displaceable over the positioning member and the cutting element between a proximal position, where the cutting element is spaced from the positioning member, and a distal position, where the cutting element and the positioning member are urged together in order to cut the leaflet therebetween.

Example 169. The cutting tool of any example herein, particularly Example 168, wherein the positioning member has a recess or gap for receiving the cutting element therein.

Example 170. The cutting tool of any example herein, particularly Example 169, wherein the recess or gap has an inverted U-shape or inverted V-shape with an open distal end, a rectangular shape with an open distal end, a U-shape or V-shape with a closed distal end, a circular shape with a closed distal end, an elliptical shape with a closed distal end, or a rectangular shape with a closed distal end.

Example 171. The cutting tool of any example herein, particularly any one of Examples 169-170, wherein the cutting element has a shape complementary to that of the recess or gap of the positioning member.

Example 172. The cutting tool of any example herein, particularly any one of Examples 168-171, wherein the cutting element has a sharp edge or comprises a blade.

Example 173. The cutting tool of any example herein, particularly any one of Examples 168-172, wherein the cutting element is configured to cut using electrical energy applied thereto.

Example 174. The cutting tool of any example herein, particularly any one of Examples 168-173, wherein the positioning member is formed of a shape-memory alloy.

Example 175. The cutting tool of any example herein, particularly any one of Examples 168-174, wherein the cutting element and/or the positioning member is configured with a snap-fit feature that couples the cutting element to the positioning member once the tubular member is displaced to the distal position.

Example 176. The cutting tool of any example herein, particularly any one of Examples 168-175, wherein a distal end of the tubular member has a diameter greater than a proximal end of the tubular member.

Example 177. The cutting tool of any example herein, particularly any one of Examples 168-176, further comprising one or more filters within the catheter and configured to be extended from the end of the catheter, each filter being constructed to capture particulate or other debris released by cutting of the leaflet.

Example 178. The cutting tool of any example herein, particularly Example 177, wherein each filter is configured to be extended radially from the end of the catheter to contact a wall of the aorta proximal of the existing valvular structure.

Example 179. A heart valve implantation method comprising:

providing a delivery system in an ascending aorta of a subject;

extending a lacerating member from a channel in the delivery system, the lacerating member being formed of a shape-memory alloy and having a distal portion that forms a hook-shape once extended from the channel, the distal portion having a sharp tip, the extending being such that the distal portion is between a free end of a leaflet of an existing valvular structure and a connection portion of the leaflet along a radial direction of the existing valvular structure, the existing valvular structure being between the ascending aorta and a left ventricle of a heart;

contacting the sharp tip of the lacerating member with the leaflet so as to pierce the leaflet between the free end and the connection portion;

cutting the leaflet by moving the lacerating member in a proximal direction along the leaflet to form a gash that splays the leaflet at the free end;

retracting the lacerating member into the channel, and removing the delivery system from the ascending aorta; and

mounting a prosthetic heart valve within the existing valvular structure.

Example 180. The heart valve implantation method of any example herein, particularly Example 179, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 181. The heart valve implantation method of any example herein, particularly Example 180, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 182. The heart valve implantation method of any example herein, particularly any one of Examples 180-181, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 183. The heart valve implantation method of any example herein, particularly any one of Examples 180-181, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 184. The heart valve implantation method of any example herein, particularly any one of Examples 179-183, further comprising, capturing particulate or debris released from the existing valvular structure by the cutting of the leaflet.

Example 185. The heart valve implantation method of any example herein, particularly Example 184, wherein the capturing is by a filter extending from the delivery system and disposed proximal of the existing valvular structure.

Example 186. The heart valve implantation method of any example herein, particularly any one of Examples 179-185, wherein the delivery system has an angled surface at its distal end with an opening for the channel along the angled surface.

Example 187. The heart valve implantation method of any example herein, particularly Example 186, wherein the providing the delivery system comprises contacting the angled surface of the delivery system with a proximal surface of the leaflet, and the contacting the sharp tip occurs while the lacerating member is being extended from the channel.

Example 188. The heart valve implantation method of any example herein, particularly any one of Examples 179-187, wherein a proximal portion and the sharp tip of the lacerating member are insulated and a portion of the hook-shape between the proximal portion and the sharp tip is non-insulated.

Example 189. The heart valve implantation method of any example herein, particularly any one of Examples 179-188, wherein the cutting comprises applying electrical energy to the lacerating member.

Example 190. The heart valve implantation method of any example herein, particularly any one of Examples 179-189, wherein the delivery system comprises a catheter.

Example 191. The heart valve implantation method of any example herein, particularly any one of Examples 179-190, wherein the channel and/or the lacerating member has a non-circular cross-section.

Example 192. The heart valve implantation method of any example herein, particularly any one of Examples 179-191, wherein:

the extending is such that a plane of the hook-shape is substantially perpendicular to the radial direction of the existing valvular structure, and

the contacting includes rotating the lacerating member such that the sharp tip of the hook-shape contacts and pierces the leaflet between the free end and the connection portion.

Example 193. The heart valve implantation method of any example herein, particularly any one of Examples 179-192, wherein the rotating the lacerating member comprises rotating the channel within the delivery system.

Example 194. The heart valve implantation method of any example herein, particularly any one of Examples 179-193, further comprising:

after the cutting and before the removing the delivery system, repositioning the delivery system or the lacerating member with respect to a different leaflet of the existing valvular structure; and

repeating the extending, contacting, and cutting for the different leaflet.

Example 195. The heart valve implantation method of any example herein, particularly any one of Examples 179-194, wherein:

the delivery system has multiple lacerating members with respective channels, each lacerating member corresponding to one of the leaflets of the existing valvular structure, and

the extending, contacting, and cutting are such that each leaflet is simultaneously cut by the respective lacerating member.

Example 196. The heart valve implantation method of any example herein, particularly any one of Examples 179-195, wherein the existing valvular structure is a native aortic valve of the heart.

Example 197. The heart valve implantation method of any example herein, particularly Example 196, wherein the gash in the splayed leaflet allows blood to flow through a frame of the prosthetic heart valve to a coronary artery of the heart, which would otherwise be blocked by the uncut leaflet of the native aortic valve.

Example 198. The heart valve implantation method of any example herein, particularly any one of Examples 196-197, wherein the native aortic valve is a bicuspid aortic valve having two leaflets, the prosthetic heart valve has a cylindrical shape, and the gash in the splayed leaflet allows the prosthetic heart valve to be mounted within the bicuspid aortic valve.

Example 199. The heart valve implantation method of any example herein, particularly any one of Examples 179-195, wherein the existing valvular structure is of a second prosthetic valve previously implanted in the subject, and the mounting comprises installing the prosthetic heart valve within the second prosthetic valve.

Example 200. The heart valve implantation method of any example herein, particularly Example 199, wherein the gash in the splayed leaflet allows blood to flow through a frame of the prosthetic heart valve en route to a coronary artery of the heart, which would otherwise be blocked by the uncut leaflet of the previously-implanted second prosthetic heart valve.

Example 201. A cutting tool for modifying a valvular structure comprising:

a delivery system configured to be disposed within an ascending aorta of a subject; and

a lacerating member extending within a channel in the delivery system and configured to cut a leaflet of an existing valvular structure,

wherein the lacerating member is formed of a shape-memory alloy and has a distal portion configured to form a hook-shape once outside the channel, the distal portion having a sharp tip.

Example 202. The cutting tool of any example herein, particularly Example 201, wherein the delivery system has an angled surface at its distal end with an opening for the channel along the angled surface.

Example 203. The cutting tool of any example herein, particularly any one of Examples 201-202, wherein a proximal portion and the sharp tip of the lacerating member are insulated, and a portion of the hook-shape between the proximal portion and the sharp tip is non-insulated.

Example 204. The cutting tool of any example herein, particularly any one of Examples 201-203, wherein the lacerating member is configured to cut using electrical energy applied thereto.

Example 205. The cutting tool of any example herein, particularly any one of Examples 201-204, wherein the channel and/or the lacerating member has a non-circular cross-section.

Example 206. The cutting tool of any example herein, particularly any one of Examples 201-205, wherein the delivery system comprises a catheter.

Example 207. The cutting tool of any example herein, particularly Example 206, wherein the channel is a rotatable channel extending within a lumen of the catheter.

Example 208. The cutting tool of any example herein, particularly Example 207, wherein the lacerating member is rotatable via the rotatable channel between a first position where a plane of the hook-shape is substantially perpendicular to a radial direction of an existing valvular structure, and a second position where the plane of the hook-shape is substantially parallel to the radial direction and the sharp tip pierces the leaflet of the existing valvular structure.

Example 209. The cutting tool of any example herein, particularly any one of Examples 201-208, further comprising one or more filters constructed to capture particulate or other debris released by cutting of the leaflet.

Example 210. The cutting tool of any example herein, particularly Example 209, wherein each filter is configured extend radially from the delivery system to contact a wall of the aorta proximal of the existing valvular structure.

Example 211. A heart valve implantation method comprising:

providing an end of a delivery shaft in an ascending aorta of a subject;

piercing a leaflet of an existing valvular structure with one or more piercing tips of a leaflet retaining device by extending the leaflet retaining device from the end of the delivery shaft;

cutting the leaflet using the leaflet retaining device;

retracting the leaflet retaining device into the delivery shaft, and removing the delivery shaft from the ascending aorta; and

mounting a prosthetic heart valve within the existing valvular structure.

Example 212. The heart valve implantation method of any example herein, particularly Example 211, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 213. The heart valve implantation method of any example herein, particularly Example 212, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 214. The heart valve implantation method of any example herein, particularly any one of Examples 212-213, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 215. The heart valve implantation method of any example herein, particularly any one of Examples 212-213, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 216. The heart valve implantation method of any example herein, particularly any one of Examples 211-215, further comprising, capturing particulate or debris released from the existing valvular structure by the cutting of the leaflet.

Example 217. The heart valve implantation method of any example herein, particularly Example 216, wherein the capturing is by a filter extending from the delivery shaft and disposed proximal of the existing valvular structure.

Example 218. The heart valve implantation method of any example herein, particularly any one of Examples 211-217, wherein the leaflet retaining device comprises a helical shape having a sharp tip at its distal end, and the piercing comprises rotating the helical shape about its axis.

Example 219. The heart valve implantation method of any example herein, particularly any one of Examples 211-217, wherein the leaflet retaining device comprises a plurality of prongs, each prong having a barbed sharp tip at its distal end.

Example 220. The heart valve implantation method of any example herein, particularly any one of Examples 211-219, wherein the cutting comprises moving the leaflet retaining device in a proximal direction to tear the leaflet.

Example 221. The heart valve implantation method of any example herein, particularly any one of Examples 211-220, wherein the end of the delivery shaft comprises a sharp or serrated cutting edge.

Example 222. The heart valve implantation method of any example herein, particularly Example 221, wherein the cutting comprises contacting the end of the delivery shaft with the leaflet pierced by the one or more piercing tips.

Example 223. The heart valve implantation method of any example herein, particularly Example 222, wherein the cutting further comprises rotating the end of delivery shaft so as to cut the leaflet.

Example 224. The heart valve implantation method of any example herein, particularly any one of Examples 211-223, further comprising:

after the cutting and before the removing the delivery shaft, repositioning the delivery shaft or the leaflet retaining device with respect to a different leaflet of the existing valvular structure; and repeating the piercing and cutting for the different leaflet.

Example 225. The heart valve implantation method of any example herein, particularly any one of Examples 211-224, wherein the existing valvular structure is a native aortic valve of the heart.

Example 226. The heart valve implantation method of any example herein, particularly Example 225, wherein the cut leaflet allows blood to flow through a frame of the prosthetic heart valve to a coronary artery of the heart, which would otherwise be blocked by the uncut leaflet of the native aortic valve.

Example 227. The heart valve implantation method of any example herein, particularly any one of Examples 225-226, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 228. The heart valve implantation method of any example herein, particularly any one of Examples 211-224, wherein the existing valvular structure is of a second prosthetic valve previously implanted in the subject, and the mounting comprises installing the prosthetic heart valve within the second prosthetic valve.

Example 229. The heart valve implantation method of any example herein, particularly Example 228, wherein the cut leaflet allows blood to flow through a frame of the prosthetic heart valve en route to a coronary artery of the heart, which would otherwise be blocked by the uncut leaflet of the previously-implanted second prosthetic valve.

Example 230. A cutting tool for modifying a valvular structure comprising:

a delivery shaft having an end configured to be disposed within an ascending aorta of a subject; and

a leaflet retaining device having one or more piercing tips, the leaflet retaining device being configured to extend from the end of the delivery shaft so as to pierce a leaflet of an existing valvular structure.

Example 231. The cutting tool of any example herein, particularly Example 230, wherein the leaflet retaining device comprises a helical shape having a sharp tip at its distal end, and the helical shape is rotatable about its axis.

Example 232. The cutting tool of any example herein, particularly Example 230, wherein the leaflet retaining device comprises a plurality of prongs, each prong having a barbed sharp tip at its distal end.

Example 233. The cutting tool of any example herein, particularly any one of Examples 230-232, wherein the end of the delivery shaft comprises a sharp or serrated cutting edge.

Example 234. The cutting tool of any example herein, particularly any one of Examples 230-233, wherein the delivery shaft is rotatable about its axis independent of the leaflet retaining device.

Example 235. The cutting tool of any example herein, particularly any one of Examples 230-234, wherein the delivery shaft comprises a catheter.

Example 236. The cutting tool of any example herein, particularly any one of Examples 230-235, further comprising one or more filters constructed to capture particulate or other debris released from the existing valvular structure.

Example 237. The cutting tool of any example herein, particularly Example 236, wherein each filter is configured to extend radially from the delivery shaft and to contact a wall of the aorta proximal of the existing valvular structure.

Example 238. A heart valve implantation method comprising:

providing a cutting tool in an ascending aorta of a subject, the cutting tool having an outer shaft and an inner shaft within the outer shaft, the inner shaft and the outer shaft being movable relative to each other along an axial direction of the outer shaft, the inner and outer shafts each having a window in a circumferential surface thereof, each window having a proximal edge and a distal edge spaced from the proximal edge along the axial direction, the window of the inner shaft overlapping with the window of the outer shaft;

positioning the cutting tool with respect to a leaflet of an existing valvular structure, the existing valvular structure being between the ascending aorta and a left ventricle of a heart, the positioning being such that a portion of the leaflet projects through the overlapping windows of the inner and outer shafts into an inner volume of the inner shaft;

cutting the leaflet by displacing one of the inner and outer shafts with respect to the other along the axial direction such that the portion of the leaflet is clamped between the distal edge of the window of the inner shaft and the proximal edge of the window of the outer shaft;

removing the cutting tool from the ascending aorta; and

mounting a prosthetic heart valve within the existing valvular structure.

Example 239. The heart valve implantation method of any example herein, particularly Example 238, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 240. The heart valve implantation method of any example herein, particularly Example 239, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 241. The heart valve implantation method of any example herein, particularly any one of Examples 239-240, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 242. The heart valve implantation method of any example herein, particularly any one of Examples 239-240, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 243. The heart valve implantation method of any example herein, particularly any one of Examples 238-242, further comprising, capturing particulate or debris released from the existing valvular structure by the cutting of the leaflet.

Example 244. The heart valve implantation method of any example herein, particularly Example 243, wherein the capturing is by a filter disposed proximal of the existing valvular structure.

Example 245. The heart valve implantation method of any example herein, particularly any one of Examples 238-244, wherein the distal edge of the window of the inner shaft is formed as one or more teeth, is a sharp cutting edge, or comprises a cutting blade.

Example 246. The heart valve implantation method of any example herein, particularly any one of Examples 238-245, wherein the proximal edge of the window of the outer shaft is a sharp cutting edge or comprises a cutting blade.

Example 247. The heart valve implantation method of any example herein, particularly any one of Examples 238-246, wherein the positioning is such that the window of the inner shaft is aligned with the window of the outer shaft.

Example 248. The heart valve implantation method of any example herein, particularly any one of Examples 238-247, wherein:

the cutting tool further comprises a cutting shaft movable relative to the outer shaft along the axial direction, the cutting shaft having a distal edge and a proximal edge, one of the distal and proximal edges of the cutting shaft being a cutting edge; and

the cutting comprises displacing the cutting shaft along the axial direction such that the cutting edge contacts and slices through the leaflet clamped by the inner and outer shafts.

Example 249. The heart valve implantation method of any example herein, particularly Example 248, wherein the proximal edge of the cutting shaft is the cutting edge, the cutting shaft is initially disposed distal of the window of the outer shaft, and the cutting comprises displacing the cutting shaft proximally.

Example 250. The heart valve implantation method of any example herein, particularly Example 248, wherein the distal edge of the cutting shaft is the cutting edge, the cutting shaft is initially disposed proximal of the window of the outer shaft, and the cutting comprises displacing the cutting shaft distally.

Example 251. The heart valve implantation method of any example herein, particularly Example 248, wherein the cutting edge of the cutting shaft is a sharp circumferential edge, a serrated circumferential edge, or a blade disposed at a circumferential edge of the cutting shaft.

Example 252. The heart valve implantation method of any example herein, particularly any one of Examples 238-251, wherein the cutting comprises applying electrical energy to the inner shaft and/or the outer shaft.

Example 253. The heart valve implantation method of any example herein, particularly any one of Examples 238-252, wherein the cut portion of the leaflet is retained within an internal lumen of the outer shaft.

Example 254. The heart valve implantation method of any example herein, particularly any one of Examples 238-253, further comprising:

after the cutting and before the removing the cutting tool, repositioning the cutting tool with respect to a different leaflet of the existing valvular structure; and

repeating the positioning and cutting for the different leaflet.

Example 255. The heart valve implantation method of any example herein, particularly any one of Examples 238-254, wherein the existing valvular structure is a native aortic valve of the heart.

Example 256. The heart valve implantation method of any example herein, particularly Example 255, wherein the cut leaflet allows blood to flow through a frame of the prosthetic heart valve to a coronary artery of the heart, which would otherwise be blocked by the uncut leaflet of the native aortic valve.

Example 257. The heart valve implantation method of any example herein, particularly any one of Examples 255-256, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 258. The heart valve implantation method of any example herein, particularly any one of Examples 238-254, wherein the existing valvular structure is of a second prosthetic valve previously implanted in the subject, and the mounting comprises installing the prosthetic heart valve within the second prosthetic valve.

Example 259. The heart valve implantation method of any example herein, particularly Example 258, wherein the cut leaflet allows blood to flow through a frame of the prosthetic heart valve en route to a coronary artery of the heart, which would otherwise be blocked by the uncut leaflet of the previously-implanted second prosthetic valve.

Example 260. A cutting tool for modifying a valvular structure comprising:

a first shaft having a first window in a circumferential surface thereof, the first window having a first proximal edge and a first distal edge spaced from the first proximal edge along an axial direction of the first shaft; and

a second shaft having a second window in a circumferential surface thereof, the second window having a second proximal edge and a second distal edge spaced from the second proximal edge along the axial direction,

wherein a diameter of the second shaft is less than a diameter of the first shaft,

at least a portion of the second shaft is disposed within the first shaft, and

at least one of the first shaft and the second shaft is movable relative to the other along the axial direction.

Example 261. The cutting tool of any example herein, particularly Example 260, wherein the second distal edge or the second proximal edge of the second window is formed as one or more teeth, is a sharp cutting edge, or comprises a cutting blade.

Example 262. The cutting tool of any example herein, particularly any one of Examples 260-261, wherein the first proximal edge or the first distal edge of the first window is a sharp cutting edge or comprises a cutting blade.

Example 263. The cutting tool of any example herein, particularly any one of Examples 260-262, wherein the first window overlaps or is aligned with the second window.

Example 264. The cutting tool of any example herein, particularly any one of Examples 260-263, further comprising a third shaft having a third distal edge and a third proximal edge, the third shaft being movable relative to the first shaft along the axial direction, at least one of the third distal edge and the third proximal edge is configured to cut a leaflet in contact therewith.

Example 265. The cutting tool of any example herein, particularly Example 264, wherein the third distal edge and/or the third proximal edge is a sharp circumferential edge, a serrated circumferential edge, or a blade disposed at a circumferential edge of the third shaft.

Example 266. The cutting tool of any example herein, particularly any one of Examples 264-265, wherein the third shaft slides over the circumferential surface of the first shaft.

Example 267. The cutting tool of any example herein, particularly any one of Examples 260-266, further comprising one or more filters constructed to capture particulate or other debris released from an existing valvular structure.

Example 268. The cutting tool of any example herein, particularly Example 267, wherein each filter is configured to extend radially from the first shaft, or a catheter that delivers the first shaft to an ascending aorta, to contact a wall of the aorta proximal of the existing valvular structure.

Example 269. A heart valve implantation method comprising:

providing a cutting tool in an ascending aorta of a subject, the cutting tool having an outer shaft and an inner shaft within the outer shaft, the inner shaft and outer shaft being movable relative to each other along axial and circumferential directions of the outer shaft, the outer shaft having a window in a circumferential surface thereof, the window having a proximal edge and a distal edge spaced from the proximal edge along the axial direction, the inner shaft having a leaflet engagement portion that is threaded, the leaflet engagement portion overlapping with the window of the outer shaft;

positioning the cutting tool with respect to a leaflet of an existing valvular structure, the existing valvular structure being between the ascending aorta and a left ventricle of a heart, the positioning being such that a portion of the leaflet projects through the window of the outer shaft to contact the threaded engagement portion;

cutting the leaflet by rotating the inner shaft within the outer shaft and/or displacing one of the inner and outer shafts with respect to the other along the axial direction, such that the portion of the leaflet is clamped between one or more threads of the engagement portion and the proximal edge of the window of the outer shaft;

removing the cutting tool from the ascending aorta; and

mounting a prosthetic heart valve within the existing valvular structure.

Example 270. The heart valve implantation method of any example herein, particularly Example 269, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 271. The heart valve implantation method of any example herein, particularly Example 270, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 272. The heart valve implantation method of any example herein, particularly any one of Examples 270-271, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 273. The heart valve implantation method of any example herein, particularly any one of Examples 270-271, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 274. The heart valve implantation method of any example herein, particularly any one of Examples 269-273, further comprising, capturing particulate or debris released from the existing valvular structure by the cutting of the leaflet.

Example 275. The heart valve implantation method of any example herein, particularly Example 274, wherein the capturing is by a filter disposed proximal of the existing valvular structure.

Example 276. The heart valve implantation method of any example herein, particularly any one of Examples 269-275, wherein the proximal edge of the window of the outer shaft is a sharp cutting edge or comprises a cutting blade.

Example 277. The heart valve implantation method of any example herein, particularly any one of Examples 269-276, wherein:

the engagement portion of the inner shaft comprises one or more sharp threads, and

the cutting comprises rotating the inner shaft such that the one or more sharp threads slice through the clamped leaflet.

Example 278. The heart valve implantation method of any example herein, particularly any one of Examples 269-277, wherein a diameter of the threaded engagement portion tapers along the axial direction.

Example 279. The heart valve implantation method of any example herein, particularly Example 278, wherein the taper is such that a diameter of the engagement portion at a distal end is larger than a diameter of the engagement portion at a proximal end.

Example 280. The heart valve implantation method of any example herein, particularly any one of Examples 269-279, wherein during the cutting, the inner shaft is rotated within the outer shaft simultaneous with displacing one of the inner and outer shafts with respect to the other along the axial direction.

Example 281. The heart valve implantation method of any example herein, particularly any one of Examples 269-280, wherein the cutting comprises using a separate cutting element between the inner and outer shafts to slice the clamped leaflet.

Example 282. The heart valve implantation method of any example herein, particularly any one of Examples 269-281, wherein the cut portion of the leaflet is retained within an internal lumen of the outer shaft.

Example 283. The heart valve implantation method of any example herein, particularly any one of Examples 269-282, further comprising:

after the cutting and before the removing the cutting tool, repositioning the cutting tool with respect to a different leaflet of the existing valvular structure; and

repeating the positioning and cutting for the different leaflet.

Example 284. The heart valve implantation method of any example herein, particularly any one of Examples 269-283, wherein the existing valvular structure is a native aortic valve of the heart.

Example 285. The heart valve implantation method of any example herein, particularly Example 284, wherein the cut leaflet allows blood to flow through a frame of the prosthetic heart valve to a coronary artery of the heart, which would otherwise be blocked by the uncut leaflet of the native aortic valve.

Example 286. The heart valve implantation method of any example herein, particularly any one of Examples 284-285, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 287. The heart valve implantation method of any example herein, particularly any one of Examples 269-283, wherein the existing valvular structure is of a second prosthetic valve previously implanted in the subject, and the mounting comprises installing the prosthetic heart valve within the second prosthetic valve.

Example 288. The heart valve implantation method of any example herein, particularly Example 287, wherein the cut leaflet allows blood to flow through a frame of the prosthetic heart valve en route to a coronary artery of the heart, which would otherwise be blocked by the uncut leaflet of the previously-implanted second prosthetic valve.

Example 289. A cutting tool for modifying a valvular structure comprising:

a first shaft having a first window in a circumferential surface thereof, the first window having a first proximal edge and a first distal edge spaced from the first proximal edge along an axial direction of the first shaft; and

a second shaft having a leaflet engagement portion that comprises one or more threads,

wherein a diameter of the second shaft is less than a diameter of the first shaft,

at least a portion of the second shaft is disposed within the first shaft,

at least one of the first shaft and the second shaft is movable relative to the other along the axial direction and along a circumferential direction of the first shaft, and

the leaflet engagement portion overlaps with the first window of the first shaft.

Example 290. The cutting tool of any example herein, particularly Example 289, wherein the first proximal edge of the first window is a sharp cutting edge or comprises a cutting blade.

Example 291. The cutting tool of any example herein, particularly any one of Examples 289-290, wherein the leaflet engagement portion comprises one or more sharp threads.

Example 292. The cutting tool of any example herein, particularly any one of Examples 289-291, wherein a diameter of the threaded leaflet engagement portion tapers along the axial direction.

Example 293. The cutting tool of any example herein, particularly Example 292, wherein the taper is such that a diameter of the leaflet engagement portion at a distal end is larger than a diameter of the leaflet engagement portion at a proximal end.

Example 294. The cutting tool of any example herein, particularly any one of Examples 289-293, further comprising a cutting element disposed between the first shaft and the second shaft.

Example 295. The cutting tool of any example herein, particularly Example 294, wherein the cutting element is configured to be rotated with respect to the first shaft and/or second shaft.

Example 296. The cutting tool of any example herein, particularly any one of Examples 289-295, further comprising one or more filters constructed to capture particulate or other debris released from an existing valvular structure.

Example 297. The cutting tool of any example herein, particularly Example 296, wherein each filter is configured to extend radially from the first shaft, or a catheter that delivers the first shaft to an ascending aorta, to contact a wall of the aorta proximal of the existing valvular structure.

Example 298. A heart valve implantation method comprising:

providing a cutting tool in an ascending aorta of a subject, the cutting tool having an outer shaft and an inner shaft within the outer shaft, the inner shaft and the outer shaft being movable relative to each other along an axial direction of the outer shaft, the outer shaft having a first window with a first proximal edge and a first distal edge spaced from the first proximal edge along the axial direction, the inner shaft having a plurality of second windows, each second window having a second proximal edge and a second distal edge spaced from the second proximal edge along the axial direction, each second distal edge having a cutting tooth, one of the second windows of the inner shaft overlapping with the first window of the outer shaft;

positioning the cutting tool with respect to a leaflet of an existing valvular structure, the existing valvular structure being between the ascending aorta and a left ventricle of a heart, the positioning being such that a portion of the leaflet projects through the overlapping first and second windows into an inner volume of the inner shaft;

cutting the leaflet by displacing one of the inner and outer shafts with respect to the other along the axial direction such that the portion of the leaflet is clamped between the cutting tooth of the second window of the inner shaft and the first proximal edge of the first window of the outer shaft;

removing the cutting tool from the ascending aorta; and

mounting a prosthetic heart valve within the existing valvular structure.

Example 299. The heart valve implantation method of any example herein, particularly Example 298, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 300. The heart valve implantation method of any example herein, particularly Example 299, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 301. The heart valve implantation method of any example herein, particularly any one of Examples 299-300, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 302. The heart valve implantation method of any example herein, particularly any one of Examples 299-300, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 303. The heart valve implantation method of any example herein, particularly any one of Examples 298-302, further comprising, capturing particulate or debris released from the existing valvular structure by the cutting of the leaflet.

Example 304. The heart valve implantation method of any example herein, particularly Example 303, wherein the capturing is by a filter disposed proximal of the existing valvular structure.

Example 305. The heart valve implantation method of any example herein, particularly any one of Examples 298-304, wherein the outer shaft has a first side in which the first window is located, the outer shaft further having a raised channel portion on the first side that extends along the axial direction.

Example 306. The heart valve implantation method of any example herein, particularly Example 305, wherein the first side faces radially outward with respect to the existing valvular structure.

Example 307. The heart valve implantation method of any example herein, particularly any one of Examples 298-306, wherein, during the cutting, a subportion of the leaflet within the channel portion is not cut by the cutting tooth of the second window.

Example 308. The heart valve implantation method of any example herein, particularly Example 307, further comprising, after the cutting:

continuing to displace one of the inner and outer shafts with respect to the other along the axial direction, thereby pulling the leaflet further into the outer shaft via the uncut subportion; and

further cutting the leaflet by displacing one of the inner and outer shafts with respect to the other along the axial direction such that another portion of the leaflet is clamped between a cutting tooth of another second window of the inner shaft and the first distal edge of the first window of the outer shaft.

Example 309. The heart valve implantation method of any example herein, particularly Example 308, wherein a distance of the cutting tooth of the another second window from a facing surface of the first shaft along a radial direction of the existing valvular structure is greater than a distance of the cutting tooth of the second window from the facing surface of the first shaft along the radial direction.

Example 310. The heart valve implantation method of any example herein, particularly any one of Examples 298-309, wherein the inner shaft and/or the outer shaft has a non-circular cross-section.

Example 311. The heart valve implantation method of any example herein, particularly any one of Examples 298-310, wherein the cut portion of the leaflet is retained within an internal lumen of the outer shaft.

Example 312. The heart valve implantation method of any example herein, particularly any one of Examples 298-311, further comprising:

after the cutting and before the removing the cutting tool, repositioning the cutting tool with respect to a different leaflet of the existing valvular structure; and

repeating the positioning and cutting for the different leaflet.

Example 313. The heart valve implantation method of any example herein, particularly any one of Examples 298-312, wherein the existing valvular structure is a native aortic valve of the heart.

Example 314. The heart valve implantation method of any example herein, particularly Example 313, wherein the cut leaflet allows blood to flow through a frame of the prosthetic heart valve to a coronary artery of the heart, which would otherwise be blocked by the uncut leaflet of the native aortic valve.

Example 315. The heart valve implantation method of any example herein, particularly any one of Examples 313-314, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 316. The heart valve implantation method of any example herein, particularly any one of Examples 298-312, wherein the existing valvular structure is of a second prosthetic valve previously implanted in the subject, and the mounting comprises installing the prosthetic heart valve within the second prosthetic valve.

Example 317. The heart valve implantation method of any example herein, particularly Example 316, wherein the cut leaflet allows blood to flow through a frame of the prosthetic heart valve en route to a coronary artery of the heart, which would otherwise be blocked by the uncut leaflet of the previously-implanted second prosthetic valve.

Example 318. A cutting tool for modifying a valvular structure comprising:

a first shaft having a first window, the first window having a first proximal edge and a first distal edge spaced from the first proximal edge along an axial direction of the first shaft; and

a second shaft having a plurality of second windows, each second window having a second proximal edge and a second distal edge spaced from the second proximal edge along the axial direction,

wherein each second distal edge has a cutting tooth,

a cross-section of the second shaft is less than a cross-section of the first shaft,

at least a portion of the second shaft is disposed within the first shaft, and

at least one of the first shaft and the second shaft is movable relative to the other along the axial direction.

Example 319. The cutting tool of any example herein, particularly Example 318, wherein:

the first window is provided on a first side of the first shaft,

a spacing of the cutting tooth for a first one of the plurality of second windows from the first side of the first shaft along a radial direction is less than a spacing of the cutting tooth for a second one of the plurality of second windows from the first side of the first shaft along the radial direction, and

the second one of the plurality of second windows is disposed distally of the first one of the plurality of second windows.

Example 320. The cutting tool of any example herein, particularly any one of Examples 318-319, wherein:

a portion of the first shaft has a fin profile, the fin profile forming a channel that extends along the axial direction, and

the fin profile portion is provided on a same side of the first shaft as the first window.

Example 321. The cutting tool of any example herein, particularly any one of Examples 318-320, wherein the first shaft and/or the second shaft have a non-circular cross-section.

Example 322. The cutting tool of any example herein, particularly any one of Examples 318-321, further comprising one or more filters constructed to capture particulate or other debris released from an existing valvular structure.

Example 323. The cutting tool of any example herein, particularly Example 322, wherein each filter is configured to extend radially from the first shaft, or a catheter that delivers the first shaft to an ascending aorta, to contact a wall of the aorta proximal of the existing valvular structure.

Example 324. A heart valve implantation method comprising:

proving an end of a delivery system in an ascending aorta of a subject;

extending a stabilizing member from the end of the delivery system to contact a surface portion of an aortic wall such that the delivery system is centered with respect to an existing valvular structure between the ascending aorta and a left ventricle of the subject's heart;

distally moving a crossing catheter from the end of the delivery system through a center of the existing valvular structure such that one or more cutting elements of the crossing catheter cut one or more leaflets of the existing valvular structure;

retracting the crossing catheter and the stabilizing member into the delivery system, and removing the delivery system from the ascending aorta; and

mounting a prosthetic heart valve within the existing valvular structure.

Example 325. The heart valve implantation method of any example herein, particularly Example 324, wherein the stabilizing member comprises a self-expanding frame or stent, or a mechanically-expandable frame or stent.

Example 326. The heart valve implantation method of any example herein, particularly any one of Examples 324-325, wherein the stabilizing member is formed of a shape-memory alloy.

Example 327. The heart valve implantation method of any example herein, particularly Example 326, wherein the stabilizing member is formed of a nickel-titanium alloy.

Example 328. The heart valve implantation method of any example herein, particularly any one of Examples 324-327, wherein the extending is such that at least a proximal end portion of the stabilizing member is retained within the delivery system.

Example 329. The heart valve implantation method of any example herein, particularly any one of Examples 324-328, wherein the extending is such that a circumference of at least a distal end portion of the stabilizing member is in contact with the aortic wall.

Example 330. The heart valve implantation method of any example herein, particularly any one of Examples 324-329, wherein the stabilizing member contacts the aortic wall at a sinotubular junction.

Example 331. The heart valve implantation method of any example herein, particularly any one of Examples 324-330, wherein each cutting element comprises a blade or sharp edge that extends radially from a central body of the crossing catheter.

Example 332. The heart valve implantation method of any example herein, particularly any one of Examples 324-331, wherein each cutting element of the crossing catheter corresponds to a respective one of the leaflets of the existing valvular structure, and more than one of the leaflets are simultaneously cut by the cutting elements during the distally moving the crossing catheter.

Example 333. The heart valve implantation method of any example herein, particularly any one of Examples 324-332, further comprising capturing particulate or debris released from the existing valvular structure by the cutting of the one or more leaflets.

Example 334. The heart valve implantation method of any example herein, particularly Example 333, wherein the capturing is by a filter extended from the end of the delivery system and disposed proximal of the existing valvular structure.

Example 335. The heart valve implantation method of any example herein, particularly any one of Examples 333-334, wherein the capturing is by a filter coupled to or part of the stabilizing member.

Example 336. The heart valve implantation method of any example herein, particularly any one of Examples 324-335, wherein the existing valvular structure is a native aortic valve of the heart.

Example 337. The heart valve implantation method of any example herein, particularly Example 336, wherein the cut one or more leaflets allow blood to flow through a frame of the prosthetic heart valve to a coronary artery of the heart, which would otherwise be blocked by the uncut one or more leaflets of the native aortic valve.

Example 338. The heart valve implantation method of any example herein, particularly any one of Examples 336-337, wherein the native aortic valve is a bicuspid aortic valve having two leaflets, the prosthetic heart valve has a cylindrical shape, and the cut one or more leaflets allow the prosthetic heart valve to be mounted within the bicuspid aortic valve.

Example 339. The heart valve implantation method of any example herein, particularly any one of Examples 324-335, wherein the existing valvular structure is of a second prosthetic valve previously implanted in the subject, and the mounting comprises installing the prosthetic heart valve within the second prosthetic valve.

Example 340. The heart valve implantation method of any example herein, particularly Example 339, wherein the cut one or more leaflets allow blood to flow through a frame of the prosthetic heart valve en route to a coronary artery of the heart, which would otherwise be blocked by the uncut one or more leaflets of the previously-implanted second prosthetic valve.

Example 341. A cutting tool for modifying a valvular structure comprising:

a delivery system configured to be disposed with an end thereof in an ascending aorta of a subject;

a stabilizing member within the delivery system and configured to be extended from the end of the delivery system to contact a surface portion of the aortic wall so as to center the delivery system with respect to an existing valvular structure; and

a crossing catheter within the delivery system and configured to be distally moved from the end of the delivery system through a center of the existing valvular structure, the crossing catheter having one or more cutting elements constructed to cut one or more leaflets of the existing valvular structure in contact therewith.

Example 342. The cutting tool of any example herein, particularly Example 341, wherein the stabilizing member comprises a self-expanding frame or stent, or a mechanically-expandable frame or stent.

Example 343. The cutting tool of any example herein, particularly any one of Examples 341-342, wherein the stabilizing member is formed of a shape-memory alloy.

Example 344. The cutting tool of any example herein, particularly Example 343, wherein the stabilizing member is formed of a nickel-titanium alloy.

Example 345. The cutting tool of any example herein, particularly any one of Examples 341-344, wherein each cutting element comprises a blade or knife edge that extends radially from a central body of the crossing catheter.

Example 346. The cutting tool of any example herein, particularly any one of Examples 341-345, wherein the crossing catheter has multiple cutting elements, each of which is positioned to cut a respective one of the leaflets of the existing valvular structure.

Example 347. The cutting tool of any example herein, particularly any one of Examples 341-346, wherein the one or more cutting elements are configured to cut using electrical energy applied thereto.

Example 348. The cutting tool of any example herein, particularly any one of Examples 341-347, further comprising one or more filters within the delivery system and configured to be extended from the end of the delivery system, each filter being constructed to capture particulate or other debris released by cutting of the one or more leaflets.

Example 349. The cutting tool of any example herein, particularly Example 348, wherein each filter is configured to be extended radially from the end of the delivery system to contact a wall of the aorta proximal of the existing valvular structure.

Example 350. The cutting tool of any example herein, particularly any one of Examples 341-347, wherein the stabilizing member comprises one or more filters or has one or more filters coupled thereto, each filter being constructed to capture particulate or other debris released by cutting of the one or more leaflets.

Example 351. A heart valve implantation method comprising:

providing an end of a sheath in an ascending aorta of a subject, the sheath having therein a torque shaft, the torque shaft having a coring tip at an end thereof, the coring tip having a cutting edge surrounding an opening;

contacting a leaflet of an existing valvular structure with the cutting edge;

cutting the leaflet using the coring tip;

applying a vacuum to the coring tip such that a cut portion of the leaflet is sucked into the coring tip via the opening;

removing the sheath from the ascending aorta; and

mounting a prosthetic heart valve within the existing valvular structure.

Example 352. The heart valve implantation method of any example herein, particularly Example 351, wherein the cutting using the coring tip comprises rotating the torque shaft about a longitudinal axis thereof.

Example 353. The heart valve implantation method of any example herein, particularly Example 352, wherein the torque shaft is rotated independently of the sheath.

Example 354. The heart valve implantation method of any example herein, particularly any one of Examples 351-353, wherein the vacuum is applied during the contacting the leaflet and the cutting the leaflet.

Example 355. The heart valve implantation method of any example herein, particularly any one of Examples 351-354, wherein the coring tip comprises a hypotube formed of a metal or metal alloy.

Example 356. The heart valve implantation method of any example herein, particularly any one of Examples 351-355, wherein the cutting edge comprises a sharpened, beveled edge or a serrated edge.

Example 357. The heart valve implantation method of any example herein, particularly any one of Examples 351-356, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 358. The heart valve implantation method of any example herein, particularly Example 357, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 359. The heart valve implantation method of any example herein, particularly any one of Examples 357-358, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 360. The heart valve implantation method of any example herein, particularly any one of Examples 357-358, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 361. The heart valve implantation method of any example herein, particularly any one of Examples 351-360, further comprising:

using a filter extended from the end of the sheath to capture particulate or debris released from the existing valvular structure by the cutting of the leaflet, the filter being disposed proximal of the existing valvular structure.

Example 362. The heart valve implantation method of any example herein, particularly any one of Examples 351-361, further comprising:

after the cutting and before the removing the sheath, repositioning the sheath or the coring tip with respect to a different leaflet of the existing valvular structure; and

repeating the contacting and the cutting for the different leaflet.

Example 363. The heart valve implantation method of any example herein, particularly Example 362, wherein the vacuum is continuously applied during the cutting, the repositioning, and the repeated contacting and cutting.

Example 364. The heart valve implantation method of any example herein, particularly any one of Examples 351-363, wherein the existing valvular structure is a native aortic valve of the heart.

Example 365. The heart valve implantation method of any example herein, particularly Example 364, wherein the cut leaflet allows blood to flow through a frame of the prosthetic heart valve to a coronary artery of the heart, which would otherwise be blocked by the uncut leaflet of the native aortic valve.

Example 366. The heart valve implantation method of any example herein, particularly any one of Examples 364-365, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 367. The heart valve implantation method of any example herein, particularly any one of Examples 351-363, wherein the existing valvular structure is of a second prosthetic valve previously implanted in the subject, and the mounting comprises installing the prosthetic heart valve within the second prosthetic valve.

Example 368. The heart valve implantation method of any example herein, particularly Example 367, wherein the cut leaflet allows blood to flow through a frame of the prosthetic heart valve en route to a coronary artery of the heart, which would otherwise be blocked by the uncut leaflet of the previously-implanted second prosthetic valve.

Example 369. A cutting tool for modifying a valvular structure comprising:

a sheath configured to be disposed within an ascending aorta of a subject; and

a coring tip disposed within the sheath and movable with respect to the sheath, the coring tip having a cutting edge surrounding an opening at an axial end thereof,

wherein the coring tip is rotatable about a longitudinal axis of the sheath.

Example 370. The cutting tool of any example herein, particularly Example 369, wherein the coring tip comprises a hypotube formed of a metal or metal alloy.

Example 371. The cutting tool of any example herein, particularly any one of Examples 369-370, wherein the cutting edge comprises a sharpened, beveled edge or a serrated edge.

Example 372. The cutting tool of any example herein, particularly any one of Examples 369-371, wherein the coring tip is coupled to an end of a torque shaft within the sheath.

Example 373. The cutting tool of any example herein, particularly Example 372, wherein the coring tip is rotatable independent of the sheath via the torque shaft.

Example 374. The cutting tool of any example herein, particularly any one of Examples 372-373, further comprising a vacuum source coupled to an internal volume of the coring tip and configured to generate a negative pressure at the opening of the coring tip.

Example 375. The cutting tool of any example herein, particularly Example 374, wherein the vacuum source is configured to apply a vacuum to a proximal end of the torque shaft.

Example 376. The cutting tool of any example herein, particularly any one of Examples 372-375, wherein the torque shaft, the coring tip, or both are part of a balloon delivery system.

Example 377. The cutting tool of any example herein, particularly any one of Examples 372-376, further comprising one or more filters constructed to capture particulate or other debris released from the valvular structure.

Example 378. The cutting tool of any example herein, particularly Example 377, wherein each filter is configured to extend radially from the sheath and to contact a wall of the aorta proximal of the valvular structure.

Example 379. A heart valve implantation method comprising:

positioning a portion of a leaflet of an existing valvular structure within a curved slot in a circumferential wall of an outer shaft of a cutting tool, the existing valvular structure being between an ascending aorta and a left ventricle of a heart of a subject, the cutting tool comprising the outer shaft, an inner shaft disposed within the outer shaft, and a nosecone abutting a distal end of the outer shaft, the inner shaft having a blade member with a cutting edge, the inner shaft being movable relative to the outer shaft;

cutting the leaflet by moving the inner shaft with respect to the outer shaft, part of the leaflet severed by the cutting being retained within the outer shaft;

removing the cutting tool from the subject with the severed part of the leaflet therein; and

mounting a prosthetic heart valve within the existing valvular structure.

Example 380. The heart valve implantation method of any example herein, particularly Example 379, wherein the positioning comprises sliding the outer shaft distally along the leaflet until a free end of the leaflet is engaged within the curved slot.

Example 381. The heart valve implantation method of any example herein, particularly any one of Examples 379-380, further comprising, prior to the positioning:

distally advancing the nosecone through the ascending aorta to a position with respect to the valvular structure, the nosecone having a guidewire lumen extending proximally therefrom; and

distally advancing the outer shaft, with the inner shaft retained therein, over the guidewire lumen into contact with the nosecone.

Example 382. The heart valve implantation method of any example herein, particularly any one of Examples 379-381, wherein the cutting comprises moving the blade member distally along an axial direction of the outer shaft while maintaining a position of the outer shaft, such that the cutting edge moves from a first location proximal of the curved slot to a second location distal of the curved slot.

Example 383. The heart valve implantation method of any example herein, particularly any one of Examples 379-382, wherein the cutting comprises rotating the blade member about a central axis thereof while maintaining a position of the outer shaft, such that the cutting edge slices through the portion of the leaflet positioned within the curved slot.

Example 384. The heart valve implantation method of any example herein, particularly any one of Examples 379-383, wherein, after the cutting and during the removing, a sidewall of the blade member blocks the curved slot and the nosecone abuts the distal end of the outer shaft, thereby enclosing the severed part of the leaflet within the outer shaft for removal with the cutting tool.

Example 385. The heart valve implantation method of any example herein, particularly any one of Examples 379-384, wherein the curved slot has a crescent-shaped profile.

Example 386. The heart valve implantation method of any example herein, particularly any one of Examples 379-385, wherein opposite ends of the curved slot are arranged along an axial direction of the outer shaft farther from the distal end of the outer shaft than a middle portion of the curved slot.

Example 387. The heart valve implantation method of any example herein, particularly any one of Examples 379-386, wherein the cutting edge of the blade member has a profile that substantially matches the profile of the curved slot.

Example 388. The heart valve implantation method of any example herein, particularly any one of Examples 379-386, wherein the cutting edge of the blade member is substantially perpendicular to the axial direction.

Example 389. The heart valve implantation method of any example herein, particularly any one of Examples 379-388, wherein the cutting edge comprises a sharpened or serrated edge.

Example 390. The heart valve implantation method of any example herein, particularly any one of Examples 379-389, further comprising:

prior to the cutting, disposing the prosthetic heart valve in a compressed state within the existing valvular structure; and

expanding the prosthetic heart valve from the compressed state to a partially-expanded state within the existing valvular structure,

wherein the mounting the prosthetic heart valve comprises, after the cutting, further expanding the prosthetic heart valve within the existing valvular structure from the partially-expanded state to a fully-expanded state.

Example 391. The heart valve implantation method of any example herein, particularly Example 390, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 392. The heart valve implantation method of any example herein, particularly any one of Examples 390-391, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 393. The heart valve implantation method of any example herein, particularly any one of Examples 390-391, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 394. The heart valve implantation method of any example herein, particularly any one of Examples 379-393, further comprising, capturing particulate or debris released from the existing valvular structure by the cutting of the leaflet.

Example 395. The heart valve implantation method of any example herein, particularly Example 394, wherein the capturing is by a filter disposed proximal of the existing valvular structure.

Example 396. The heart valve implantation method of any example herein, particularly any one of Examples 379-395, further comprising:

after the cutting and before the removing the cutting tool, repositioning the cutting tool with respect to a different leaflet of the existing valvular structure; and

repeating the positioning and cutting for the different leaflet.

Example 397. The heart valve implantation method of any example herein, particularly any one of Examples 379-396, wherein the existing valvular structure is a native aortic valve of the heart.

Example 398. The heart valve implantation method of any example herein, particularly Example 397, wherein the cut leaflet allows blood to flow through a frame of the prosthetic heart valve to a coronary artery of the heart, which would otherwise be blocked by the uncut leaflet of the native aortic valve.

Example 399. The heart valve implantation method of any example herein, particularly any one of Examples 397-398, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 400. The heart valve implantation method of any example herein, particularly any one of Examples 379-399, wherein the existing valvular structure is of a second prosthetic valve previously implanted in the subject, and the mounting comprises installing the prosthetic heart valve within the second prosthetic valve.

Example 401. The heart valve implantation method of any example herein, particularly Example 400, wherein the cut leaflet allows blood to flow through a frame of the prosthetic heart valve en route to a coronary artery of the heart, which would otherwise be blocked by the uncut leaflet of the previously-implanted second prosthetic valve.

Example 402. A cutting tool for modifying a valvular structure, comprising:

an outer shaft having a circumferential wall with a curved slot therein;

an inner shaft disposed within and movable with respect to the outer shaft, the inner shaft having a blade member with a cutting edge; and

a nosecone having a proximal end that abuts a distal end of the outer shaft,

wherein the curved slot is constructed to receive therein a portion of a leaflet of the valvular structure, and the blade member is constructed to cut the leaflet in the curved slot by moving with respect to the outer shaft.

Example 403. The cutting tool of any example herein, particularly Example 402, wherein the nosecone has a guidewire lumen extending proximally therefrom, and the inner and outer shafts are coaxial with the guidewire lumen.

Example 404. The cutting tool of any example herein, particularly any one of Examples 402-403, wherein the blade member is constructed to cut the leaflet by moving along an axial direction of the outer shaft, by rotating about a central axis of the blade member, or both.

Example 405. The cutting tool of any example herein, particularly any one of Examples 402-404, wherein the blade member has a sidewall disposed so as to block the curved slot after the blade member is displaced distal of the curved slot to perform the cutting of the leaflet.

Example 406. The cutting tool of any example herein, particularly any one of Examples 402-405, wherein the curved slot has a crescent-shaped profile.

Example 407. The cutting tool of any example herein, particularly any one of Examples 402-406, wherein opposite ends of the curved slot are arranged along an axial direction of the outer shaft farther from the distal end of the outer shaft than a middle portion of the curved slot.

Example 408. The cutting tool of any example herein, particularly any one of Examples 402-407, wherein the cutting edge of the blade member has a profile that substantially matches the profile of the curved slot.

Example 409. The cutting tool of any example herein, particularly any one of Examples 402-407, wherein the cutting edge of the blade member is substantially perpendicular to the axial direction.

Example 410. The cutting tool of any example herein, particularly any one of Examples 402-409, wherein the cutting edge comprises a sharpened or serrated edge.

Example 411. The cutting tool of any example herein, particularly any one of Examples 402-410, further comprising one or more filters constructed to capture particulate or other debris released from an existing valvular structure.

Example 412. The cutting tool of any example herein, particularly Example 411, wherein each filter is configured to extend radially from the outer shaft, or a catheter that delivers the outer shaft to an ascending aorta, to contact a wall of the aorta proximal of the existing valvular structure.

Example 413. A heart valve implantation method comprising:

disposing a prosthetic heart valve in a compressed state within an existing valvular structure, the existing valvular structure being between the ascending aorta and a left ventricle of a subject;

expanding the prosthetic heart valve from the compressed state to a partially-expanded state, such that leaflets of the existing valvular structure are displaced radially outward and positioned in an annular region surrounding the prosthetic heart valve;

using a cutting tool, cutting at least one of the leaflets positioned within the annular region; and

after the cutting, mounting the prosthetic heart valve within the existing valvular structure by further expanding the prosthetic heart valve from the partially-expanded state to a fully-expanded state.

Example 414. The heart valve implantation method of any example herein, particularly Example 413, wherein the expanding the prosthetic heart valve from the compressed state to the partially-expanded state comprises:

actuating one or more actuators coupled to a frame of the prosthetic heart valve; and

after the actuating, locking the one or more actuators to hold the prosthetic heart valve in the partially-expanded state.

Example 415. The heart valve implantation method of any example herein, particularly any one of Examples 413-414, wherein the further expanding the prosthetic heart valve from the partially-expanded state to the fully-expanded state comprises:

further actuating the one or more actuators coupled to the frame of the prosthetic heart valve; and

after the further actuating, locking the one or more actuators to hold the prosthetic heart valve in the fully-expanded state.

Example 416. The heart valve implantation method of any example herein, particularly any one of Examples 413-414, wherein the further expanding the prosthetic heart valve from the partially-expanded state to the fully-expanded state comprises:

disposing a balloon catheter within the prosthetic heart valve, the balloon catheter being disposed in a collapsed state and the prosthetic heart valve being in the partially-expanded state; and

inflating the balloon catheter from the collapsed state to an expanded state, thereby expanding the prosthetic heart valve to the fully-expanded state.

Example 417. The heart valve implantation method of any example herein, particularly any one of Examples 413-416, further comprising, capturing particulate or debris released from the existing valvular structure by the cutting the at least one of the leaflets.

Example 418. The heart valve implantation method of any example herein, particularly Example 417, wherein the capturing is by a filter disposed proximal of the existing valvular structure.

Example 419. The heart valve implantation method of any example herein, particularly any one of Examples 413-418, wherein the existing valvular structure is a native aortic valve of the heart, and the annular region is formed between an outer circumferential surface of the prosthetic heart valve and a native aortic wall or annulus.

Example 420. The heart valve implantation method of any example herein, particularly Example 419, wherein the cut leaflet allows blood to flow through a frame of the prosthetic heart valve to a coronary artery of the heart, which would otherwise be blocked by the uncut leaflet of the native aortic valve.

Example 421. The heart valve implantation method of any example herein, particularly any one of Examples 419-420, wherein the native aortic valve is a bicuspid aortic valve having two leaflets, the prosthetic heart valve has a cylindrical shape, and the cut leaflet allows the prosthetic heart valve to be mounted within the bicuspid aortic valve.

Example 422. The heart valve implantation method of any example herein, particularly any one of Examples 413-418, wherein the existing valvular structure is of a second prosthetic valve previously implanted in the subject, the annular region is formed between an outer circumferential surface of the prosthetic heart valve and an inner circumferential surface of the previously-implanted second prosthetic valve, and the mounting comprises installing the prosthetic heart valve within the second prosthetic valve.

Example 423. The heart valve implantation method of any example herein, particularly Example 422, wherein the cut leaflet allows blood to flow through a frame of the prosthetic heart valve en route to a coronary artery of the heart, which would otherwise be blocked by the uncut leaflet of the previously-implanted second prosthetic valve.

Example 424. The heart valve implantation method of any example herein, particularly any one of Examples 413-423, wherein a delivery system is used to deliver the prosthetic heart valve to the existing valvular structure, and the same delivery system is used to deliver the tool to the existing valvular structure after the expanding the prosthetic heart valve to the partially-expanded state.

Example 425. The heart valve implantation method of any example herein, particularly any one of Examples 413-423, wherein a first delivery system is used to deliver the prosthetic heart valve to the existing valvular structure, and a second delivery system, different from the first delivery system, is used to deliver the tool to the existing valvular structure.

Example 426. The heart valve implantation method of any example herein, particularly any one of Examples 413-425, wherein said cutting tool comprises the cutting tool of any example herein, particularly any one of Examples 21-29, 47-54, 76-85, 105-113, 133-144, 168-178, 201-210, 230-237, 260-268, 289-297, 318-323, 341-350, 369-378, and 402-412.

Example 427. The heart valve implantation method of any example herein, particularly any one of Examples 1-20, 31-46, 55-75, 86-104, 114-132, 145-167, 179-200, 211-229, 238-259, 269-288, 298-317, 324-340, 351-368, 379-401, 413-426, wherein the subject is a medical patient, an animal model, a cadaver, and or a simulator of the cardiac and vasculature system.

Example 428. The heart valve implantation method of any example herein, particularly any one of Examples 1-20, 31-46, 55-75, 86-104, 114-132, 145-167, 179-200, 211-229, 238-259, 269-288, 298-317, 324-340, 351-368, 379-401, 413-426, wherein the method is performed as a training or a practice procedure within a cadaver or a simulator of the cardiac and vasculature system.

### CONCLUSION

All features described herein are independent of one another and, except where structurally impossible, can be used in combination with any other feature described herein. In view of the many possible embodiments to which the principles of the disclosed technology may be applied, it should be recognized that the illustrated embodiments are only preferred examples and should not be taken as limiting the scope of the disclosed technology. Rather, the scope is defined by the following claims. We therefore claim all that comes within the scope and spirit of these claims.

1. A cutting tool for modifying a valvular structure, comprising:
a catheter having an end configured to be disposed within an ascending aorta of a patient;
a first spacing member within the catheter and configured to be extended from the end of the catheter to contact a leaflet of an existing valvular structure;
a second spacing member within the catheter and configured to be extended from the end of the catheter to contact a structure opposite the leaflet along a radial direction; and
a lacerating member within the catheter and configured to be extended from the end of the catheter to contact and cut the leaflet in a region between a free end of the leaflet and the first spacing member.

2. The cutting tool of claim 1, wherein the lacerating member is configured to cut using electrical energy applied thereto.

3. The cutting tool of claim 1, wherein the lacerating member extending from the end of the catheter has a hook-shape with a sharp tip.

4. The cutting tool of claim 1, wherein the first spacing member, the second spacing member, or the lacerating member comprises a wire formed of a shape memory alloy.

5. (canceled)

6. (canceled)

7. The cutting tool of claim 1, further comprising one or more filters within the catheter and configured to be extended from the end of the catheter, each filter being constructed to capture particulate or other debris released by cutting of the leaflet.

8. (canceled)

9. The cutting tool of claim 1, wherein the end of the catheter has a multi-channel positioning member with channels through which the first spacing member, the second spacing member, and the lacerating member respectively extend, and the multi-channel positioning member is configured to maintain relative positions between the first spacing member, the second spacing member, and the lacerating member during rotation of the multi-channel positioning member within the ascending aorta.

10. A cutting tool for modifying a valvular structure, comprising:
a catheter having an end configured to be disposed within an ascending aorta of a patient; and
a head portion coupled to the end of the catheter,
wherein the head portion has a wall with a first recess and a second recess on an opposite side from the first recess,
the first recess has a cutting element at an edge thereof and being configured to cut a commissure of an existing valvular structure inserted into the first and second recesses, and
the second recess is configured to grip the commissure inserted into the first and second recesses.

11. (canceled)

12. The cutting tool of claim 10, wherein a shape of the first recess is different than a shape of the second recess.

13. The cutting tool of claim 10, further comprising a guidewire that extends through a lumen of the catheter and is configured to orient itself within the commissure.

14. (canceled)

15. A cutting tool for modifying a valvular structure, comprising:
a delivery system configured to be disposed within an ascending aorta of a patient; and
a lacerating member extending within a channel in the delivery system and configured to cut a leaflet of an existing valvular structure,
wherein the lacerating member is formed of a shape-memory alloy and has a distal portion configured to form a hook-shape once outside the channel, the distal portion having a sharp tip.

16. (canceled)

17. A cutting tool for modifying a valvular structure comprising:
a delivery shaft having an end configured to be disposed within an ascending aorta of a patient; and
a leaflet retaining device having one or more piercing tips, the leaflet retaining device being configured to extend from the end of the delivery shaft so as to pierce a leaflet of an existing valvular structure.

18. A cutting tool for modifying a valvular structure comprising:
a delivery system configured to be disposed with an end thereof in an ascending aorta of a patient;
a stabilizing member within the delivery system and configured to be extended from the end of the delivery system to contact a surface portion of an aortic wall so as to center the delivery system with respect to an existing valvular structure; and
a crossing catheter within the delivery system and configured to be distally moved from the end of the delivery system through a center of the existing valvular structure, the crossing catheter having one or more cutting elements constructed to cut one or more leaflets of the existing valvular structure in contact therewith.

19. A cutting tool for modifying a valvular structure comprising:
a sheath configured to be disposed within an ascending aorta of a patient; and
a coring tip disposed within the sheath and movable with respect to the sheath, the coring tip having a cutting edge surrounding an opening at an axial end thereof,
wherein the coring tip is rotatable about a longitudinal axis of the sheath.

20. A cutting tool for modifying a valvular structure, comprising:
an outer shaft having a circumferential wall with a curved slot therein;
an inner shaft disposed within and movable with respect to the outer shaft, the inner shaft having a blade member with a cutting edge; and
a nosecone having a proximal end that abuts a distal end of the outer shaft,
wherein the curved slot is constructed to receive therein a portion of a leaflet of the valvular structure, and the blade member is constructed to cut the leaflet in the curved slot by moving with respect to the outer shaft.

21. A heart valve implantation method comprising:
disposing a prosthetic heart valve in a compressed state within an existing valvular structure, the existing valvular structure being between an ascending aorta and a left ventricle of a subject;
expanding the prosthetic heart valve from the compressed state to a partially-expanded state, such that leaflets of the existing valvular structure are displaced radially outward and positioned in an annular region surrounding the prosthetic heart valve;
using a cutting tool, cutting at least one of the leaflets positioned within the annular region; and
after the cutting, mounting the prosthetic heart valve within the existing valvular structure by further expanding the prosthetic heart valve from the partially-expanded state to a fully-expanded state.

22. The heart valve implantation method of claim 21, wherein the expanding the prosthetic heart valve from the compressed state to the partially-expanded state comprises:
actuating one or more actuators coupled to a frame of the prosthetic heart valve; and
after the actuating, locking the one or more actuators to hold the prosthetic heart valve in the partially-expanded state.

23. The heart valve implantation method of claim 21 wherein the further expanding the prosthetic heart valve from the partially-expanded state to the fully-expanded state comprises:
further actuating one or more actuators coupled to a frame of the prosthetic heart valve; and
after the further actuating, locking the one or more actuators to hold the prosthetic heart valve in the fully-expanded state.

24. The heart valve implantation method of claim 21, wherein the further expanding the prosthetic heart valve from the partially-expanded state to the fully-expanded state comprises:
disposing a balloon catheter within the prosthetic heart valve, the balloon catheter being disposed in a collapsed state and the prosthetic heart valve being in the partially-expanded state; and
inflating the balloon catheter from the collapsed state to an expanded state, thereby expanding the prosthetic heart valve to the fully-expanded state.

25. (canceled)

26. (canceled)

27. The heart valve implantation method of claim 21, wherein the existing valvular structure is a native aortic valve of the heart, and the annular region is formed between an outer circumferential surface of the prosthetic heart valve and a native aortic wall or annulus.

28. (canceled)

29. (canceled)

30. The heart valve implantation method of claim 21, wherein the existing valvular structure is of a second prosthetic valve previously implanted in the subject.

31. (canceled)
